(19) United States
(12) Patent Application Publication
(10) Pub. No.: US 2021/0158924 A1
(43) Pub. Date: May 27, 2021

(54) MANAGING HEALTHCARE SERVICES

(71) Applicant: ABBVIE BIOTECHNOLOGY LTD., Hamilton (BM)

(72) Inventors: Peter Carl Stueckemann, Libertyville, IL (US); Pankaj Dubey, Lake Villa, IL (US); Richard Lanier, Inverness, IL (US); Prakash Venkataramanan, Lake Villa, IL (US); Vaibhav Jindal, Waukegan, IL (US); Shannon Marie Sword, Gurnee, IL (US)

(21) Appl. No.: 17/141,500

(22) Filed: Jan. 5, 2021

Related U.S. Application Data
(63) Continuation of application No. 16/145,764, filed on Sep. 28, 2018, now abandoned.
Application No. 16/145,764 is a continuation of application No. 14/107,154, filed on Dec. 16, 2013, now abandoned.
Application No. 14/107,154 is a continuation of application No. 13/649,070, filed on Oct. 10, 2012, now abandoned.
(60) Provisional application No. 61/623,032, filed on Apr. 11, 2012.
(60) Provisional application No. 61/622,930, filed on Apr. 11, 2012.
(60) Provisional application No. 61/545,480, filed on Oct. 10, 2011.

(51) Int. Cl. G16H 20/10; G16Z 99/00; G16H 40/60; G16H 15/00

(52) U.S. Cl. CPC G16H 20/10; G16Z 99/00; G06Q 10/10; G16H 15/00; G16H 40/60

(57) ABSTRACT

System and method for facilitating a medical order/prescription of a prescription product, including a memory device to store predefined forms for the prescription product corresponding to a plurality of providers. A receiver receives prescription product information for the prescription product, patient intake information for the patient, including provider information for the patient, and a benefits summary related to the patient. A transmitter transmits the benefits verification request. A processor can be configured to generate the benefits verification request for the patient based on the patient intake information, select one of the predefined forms based on at least the patient provider information, populate at least one field of the selected predefined form based on the user intake information, and release the populated predefined form to facilitate a medical order/prescription of the prescription product to the patient.

### CROSS-REFERENCE TO RELATED APPLICATIONS

This application is a continuation application of U.S. patent application Ser. No. 16/145,764, filed on Sep. 28, 2018, which is a continuation of U.S. patent application Ser. No. 14/107,154, filed on Dec. 16, 2013, which is a continuation application of U.S. patent application Ser. No. 13/649,070, filed on Oct. 10, 2012, which claims priority to U.S. Provisional Application Ser. No. 61/623,032, filed Apr. 11, 2012, U.S. Provisional Application Ser. No. 61/622,930, filed Apr. 11, 2012, and U.S. Provisional Application Ser. No. 61/545,480, filed Oct. 10, 2011, each of which is incorporated herein by reference in its entirety.

### BACKGROUND

The disclosed subject matter relates to healthcare services and, more particularly, to facilitating, coordinating, or managing healthcare products and/or services, such as pharmaceutical products, drugs, medical devices, or other prescribed medical treatments.

When a patient is consulting with a healthcare providers (HCP), such as a doctor or a nurse practitioner, the healthcare providers may prescribe a specific healthcare product or service to the patient (e.g., as a part of the patient's diagnosis or treatment). As an example, the healthcare providers may prescribe a medication (e.g., a pharmaceutical or biologic product) or a medical device (e.g., an oxygen cart) to the patient. As another example, the healthcare providers may refer the patient to another healthcare provider who is a specialist in a specific field (e.g., a general practice doctor may refer the patient to a cardiologist).

If the patient has medical insurance, sometimes, the patient's medical insurance provider may be obligated to pay for part or all of the cost of the product or service the healthcare providers has prescribed to the patient. However, for some types of prescription products or services, an insurance provider may require a specific type of authorization or approval, often referred to as “prior authorization” (PA), before it is willing to pay for the products or services prescribed to a patient.

Some prescription product sellers (e.g., pharmacies) or prescription service providers permit a healthcare provider to transmit a prescription electronically for a product or service to the product sellers or service providers (e.g., via fax, electronic prescription, electronic document sharing site, file transfer protocol (FTP) site, electronic transmission, transmission of an image, or electronic mail (email)). For example, a doctor may electronically transmit a prescription for a pharmaceutical drug to a pharmacy to be filled. Upon receiving insurance information from the patient for whom the pharmaceutical prescription is written, the pharmacy may need to contact the patient's insurance provider to determine if the insurance provider requires prior authorization for the prescribed pharmaceutical drug before the insurance provider agrees to pay for the drug. As used herein, the term pharmaceutical prescription shall be understood to include drugs, pharmaceutical products, medical devices, medical therapies as well as other products that require a prescription from a licensed HCP. Furthermore, a healthcare provider may issue a medical order, such as for a treatment or the like. If PA is required, the patient's insurance provider sends the appropriate PA form to the HCP who has written the prescription and the HCP completes and signs the form. The HCP then transmits or sends the completed PA form or request for products and/or services to the patient's insurance provider. Upon receipt and approval of the PA form by the patient's insurance provider, the insurance provider transmits an approval of benefits to the pharmacy, at which time the pharmacy may fill the prescription and dispense the product to the patient.

Such a process for obtaining prior authorization for a prescription product or service is inconvenient, time consuming, and requires numerous people to process and transfer the necessary paperwork as well as potentially exposes multiple people to the patient's confidential health information. Due to this complex system, the risk arises that prescriptions may go unfilled due to lost paperwork, or lack of access to financial or training materials. Thus, under the current system, many patients today may not have access to necessary medical treatment.

Moreover, various services and/or benefits may be available to a patient from third parties. If the patient is unaware of these services and benefits, the patient may not be able to take advantage of such available benefits and may not fulfill the prescription due to financial constraints or lack of understanding on how to use the product and/or service. Further, the third parties may not be able to directly contact the patient without the patient's prior consent.

### SUMMARY

In one aspect of the disclosed subject matter, a system for facilitating a medical order/prescription of a prescription product for a patient covered by a provider includes at least one memory device to store a plurality of predefined forms for the prescription product. The plurality of predefined forms can correspond to a plurality of providers. The system includes a receiver to receive prescription product information for the prescription product and patient intake information for the patient. The patient intake information comprises provider information for the patient. The receiver further receives a benefits summary related to the patient in response to a benefits verification request. The system includes a transmitter to transmit the benefits verification request, and a processor configured to generate the benefits verification request for the patient based on the patient intake information. The processor is also configured to select one of the predefined forms based on at least the patient provider information, populate at least one field of the selected predefined form based on the user intake information, and release the populated predefined form to facilitate the medical order/prescription of the prescription product to the patient.

As embodied herein, for illustration, facilitating the medical order/prescription can include facilitating a prescription of the prescription product or facilitating approval of a payment for the prescription product. The provider can include an insurance carrier, a governmental agency, or other third party payor. The prescription product can include a medical product, a medical service, or an administration of a medial product. The prescription product can include a biologic product, such as adalimumab. The predefined forms can include at least one prior authorization form. Additionally, the at least one memory device can store a second plurality of predefined forms for a second prescription product, the second plurality of predefined forms corresponding to a plurality of providers. In an exemplary embodiment, the processor can be configured to automatically select one of the predefined forms based on the patient provider information.

Furthermore, as embodied herein, for illustration, the transmitter can be configured to transmit the benefits verification request to a benefits verifier, and the receiver can be configured to receive the benefits verification summary from the benefits verifier. In an exemplary embodiment, the receiver can be configured to receive information about the predefined forms from the benefits verifier, and the processor can be configured to select one of the predefined forms based on the patient provider information and further based on the information about the predefined forms received from the benefits verifier. The transmitter can be further be configured to transmit a request for additional patient information, and the processor can be further configured to receive the additional patient information and populate at least one field of the selected predefined form with the additional patient information. The additional patient information can include information required for the selected predefined form and not included in the patient intake information or the prescription product information for the prescription product. The processor can be configured to release the populated predefined form to the provider of the patient. The processor can further be configured to generate a prescription document from at least a portion of the prescription product information and the patient intake information, and release the prescription document to a pharmacy.

As embodied herein, for illustration, the system can further include at least one user device to introduce the patient intake information and prescription product information to the receiver. In an exemplary embodiment, the transmitter and receiver can be connected to a network, and the transmitter can be configured to transmit markup language describing a user interface over the network. The user interface can include fields for entry of the patient intake information and the prescription product information.

A user device, such as a tablet, mobile phone, or laptop, can be connected to the network, and can include a memory for storing data and a processor configured to parse the markup language and display the user interface, store in the memory the patient intake information and prescription product information entered into the fields of the user interface, and transmit the patient intake information and prescription product information to the receiver. In an exemplary embodiment, the processor of the user device can be further configured to receive a healthcare provider signature and generate a prescription document from the prescription product information. Additionally, the processor of the user device can be configured to transmit the prescription documents to a pharmacy. In an exemplary embodiment, the system can further include a scanning device communicatively coupled to the at least one user device, the processor of which can be configured to receive one or more images of a patient information document, such as a license or a medical insurance card, from the scanning device, extract at least part of the patient intake information from the image of the patient information document, and automatically populate at least one field of the user interface.

In another aspect of the disclosed subject matter, a method for facilitating a medical order/prescription of a prescription product for a patient covered by a provider includes providing at least one memory having stored therein a plurality of predefined forms for the prescription product, the plurality of predefined forms corresponding to a plurality of providers. The method includes receiving patient intake information comprising provider information of the patent and prescription product information for the prescription product and generating, by a processor, a benefits verification request for the patient based on the patient intake information. A benefits summary can be obtained based on the benefits verification request. One of the predefined forms can be selected, by a processor, based on at least the patient provider information and the benefits summary, and at least one field of the selected predefined form can be populated based on the patient information. The method includes facilitating the medical order/prescription of the prescription product to the patient with the selected predefined form.

Furthermore, as embodied herein, facilitating the medical order/prescription can include facilitating a prescription of the prescription product or facilitating approval of a payment for the prescription product. The provider can include an insurance carrier, a governmental agency, or other third party payor. The prescription product can include a medical product, a medical service, or an administration of a medial product. The prescription product can include a biologic product, such as adalimumab. The predefined forms can include at least one prior authorization form. Additionally, the method can further include selecting a prescription product from a number of possible prescription products, the memory having stored therein a plurality of predefined forms for each possible prescription product. In an exemplary embodiment, selecting one of the predefined forms can include automatically selecting, by the processor, one of the predefined forms.

As embodied herein, for illustration, obtaining the benefits summary can include transmitting the benefits verification request to a benefits verifier and receiving the benefits summary from the benefits verifier. In an exemplary embodiment, the method can include receiving information about the predefined forms from the benefits verifier, and selecting one of the predefined forms based on the information about the predefined forms received from the benefits verifier. In an exemplary embodiment, the method can further include requesting additional patient information. Additionally, additional patient information can be received and at least one empty field of the selected predefined form can be populated with the additional patient information. The additional patient information can include information required for the selected predefined form and not included in the patient intake information or prescription product information for the prescription product. The populated predefined form can be released to the provider of the patient.

As embodied herein, for illustration, the method can further include receiving a healthcare provider signature, generating a prescription document from the prescription product information, and releasing the prescription document to a pharmacy. In an exemplary embodiment, method can include transmitting markup language describing a user interface over a network. The user interface can include fields for entry of the patient intake information and the prescription product information.

As further embodied herein, for illustration, the method can include, at a user device, such as a tablet, mobile phone, laptop, or desktop computer, parsing the markup language and displaying the user interface, storing in the memory the patient intake information and prescription product information entered into the fields of the user interface, and transmitting the patient intake information and prescription product information to the receiver. Additionally or alternatively, the method can include, at the user device, generating a prescription document from at least a portion of the prescription product information and the patient intake information, and receiving a healthcare provider signature prior to generating the prescription document. The prescription document can be transmitted to a pharmacy. Additionally or alternatively, the method can include, at the user device, receiving one or more images of a patient identification document, such as a license or an insurance card, from a scanning device communicatively coupled to the device, extracting at least part of the patient intake information from the image of the patient identification document, and automatically populating at least one field of the user interface.

In another aspect of the disclosed subject matter, a non-transitory computer readable medium contains computer-executable instructions that when executed cause one or more user devices to perform a method of facilitating the medical order/prescription of a prescription product for a patient covered by a provider.

It is to be understood that both the foregoing general description and the following detailed description are exemplary and are intended to provide further explanation of the disclosed subject matter.

The accompanying drawings, which are incorporated in and constitute part of this specification, are included to illustrate and provide further understanding of the disclosed subject matter. It will be appreciated that the drawings are not to scale, and are provided for purposes of illustration only. Together with the description, the drawings serve to explain the principles of the disclosed subject matter.

### BRIEF DESCRIPTION OF THE DRAWINGS

FIG. 1 is a block diagram of an exemplary system for facilitating the medical order/prescription of a prescription product in accordance with an embodiment of the disclosed subject matter.

FIG. 2 is a flow diagram of an exemplary method for facilitating the medical order/prescription of a prescription product in accordance with an embodiment of the disclosed subject matter.

FIG. 3A is a block diagram of a server architecture in accordance with an embodiment of the disclosed subject matter.

FIG. 3B is another block diagram of a server architecture in accordance with an embodiment of the disclosed subject matter.

FIG. 3C illustrates an exemplary configuration of a server computer device in accordance with an embodiment of the disclosed subject matter.

FIG. 4 illustrates an exemplary configuration of a user device in accordance with an embodiment of the disclosed subject matter.

FIGS. 5A and 5B illustrate exemplary embodiments of a user device in accordance with embodiments of the disclosed subject matter.

FIG. 6 is a block diagram of an exemplary system for facilitating the medical order/prescription of a prescription product in accordance with another embodiment of the disclosed subject matter.

FIG. 7 illustrates an example method for obtaining insurance authorizations on prescription products or services.

FIGS. 8-47, including FIGS. 22B, 24B, 33B, 33C, 39B, 44B, 45B-45G, 46B, and 47B, are exemplary screenshots of embodiments of a computer program implementing the systems and methods of the disclosed subject matter.

FIGS. 48-65 are exemplary screenshots of other embodiments of a computer program implementing the systems and methods of the disclosed subject matter.

FIG. 66 is a flow block diagram of a drug medical order/prescription management system in accordance with an embodiment of the disclosed subject matter.

FIG. 67 is a flow diagram of a patient intake process using the drug medical order/prescription management system of FIG. 66.

FIG. 68 is a flow diagram of a patient opt-in process in accordance with an embodiment of the disclosed subject matter.

FIG. 69 is a flow diagram of a process configured to generate a benefit verification (BV) and E-Prescription in accordance with one embodiment of the disclosed subject matter.

FIG. 70 is a flow diagram of a prior authorization (PA) process in accordance with an embodiment of the disclosed subject matter.

FIG. 71 is a flow diagram of administrator activities to register a facility, staff member, and physician into the drug medical order/prescription management system of FIG. 66 in accordance with an embodiment of the disclosed subject matter.

FIG. 72 is a system diagram of a computer system in accordance with an embodiment of the disclosed subject matter

FIG. 73 is an exemplary screenshot of a registration window of an embodiment in accordance with the disclosed subject matter.

FIG. 74A-74E are a diagrammatical map of an exemplary computer program implementing the system and methods of an embodiment in accordance with the disclosed subject matter.

FIG. 75A-75F are a diagrammatical map of another exemplary computer program implementing the system and methods of an embodiment in accordance with the disclosed subject matter.

FIG. 76A-81B are exemplary screenshots of another embodiment of a computer program implementing the systems and methods of the disclosed subject matter.

Throughout the drawings, the same reference numerals and characters, unless otherwise stated, are used to denote like features, elements, components or portions of the illustrated embodiments. Moreover, while the disclosed subject matter will now be described in detail with reference to the figures, it is done so in connection with the illustrative embodiments.

### DETAILED DESCRIPTION

The disclosed subject matter relates to techniques for facilitating the medical order/prescription of a prescription product for a patient covered by a provider. The purpose and advantages of the disclosed subject matter will be set forth and apparent from the description that follows. Additional advantages of the disclosed subject matter will be realized and attained by the methods, apparatus, and devices particularly pointed out in the written description and claims thereof, as well as from the appended drawings.

Sometimes, when a patient is visiting and consulting with a healthcare providers (HCP) (e.g., a doctor, a nurse practitioner, or the like) and the healthcare providers prescribes/orders a prescription product (e.g., a prescription medication) and/or service (e.g., a treatment, a referral to a specialist) to the patient, the insurance provider of the patient may require prior authorization for the prescription product or service before the insurance provider agrees to pay for a part or all of the cost of the prescription product or service. To obtain the necessary authorization from the insurance provider, the healthcare providers may need to fill and sign a predefined form, such as a prior authorization form, and send the form to the insurance provider of the patient. The authorization form may include fields for various pieces of information concerning the patient, the prescription product and/or service, and the healthcare provider. The insurance provider may then decide whether it is willing to pay for the prescribed product and/or service based on the information submitted in the authorization form. If the insurance provider approves the prior authorization for the prescription product and/or service, the insurance provider may send an approval of benefits in response.

In accordance with the disclosed subject matter herein, the system for facilitating a medical order/prescription of a prescription product for a patient covered by a provider generally includes at least one memory device to store a plurality of predefined forms for the prescription product. The plurality of predefined forms can correspond to a plurality of providers. The system includes a receiver to receive prescription product information for the prescription product and patient intake information for the patient. The patient intake information comprises provider information for the patient. The receiver further receives a benefits summary related to the patient in response to a benefits verification request. The system includes a transmitter to transmit the benefits verification request, and a processor configured to generate the benefits verification request for the patient based on the patient intake information. The processor is also configured to select one of the predefined forms based on at least the patient provider information, populate at least one field of the selected predefined form based on the user intake information, and release the populated predefined form to facilitate the medical.

In accordance with the disclosed subject matter, the method for facilitating a medical order/prescription of a prescription product for a patient covered by a provider generally includes providing at least one memory having stored therein a plurality of predefined forms for the prescription product, the plurality of predefined forms corresponding to a plurality of providers. The method includes receiving patient intake information comprising provider information of the patent and prescription product information for the prescription product and generating, by a processor, a benefits verification request for the patient based on the patient intake information. A benefits summary can be obtained based on the benefits verification request. One of the predefined forms can be selected, by a processor, based on at least the patient provider information and the benefits summary, and at least one field of the selected predefined form can be populated based on the patient information. The method includes facilitating the medical order/prescription of the prescription product to the patient with the selected predefined form.

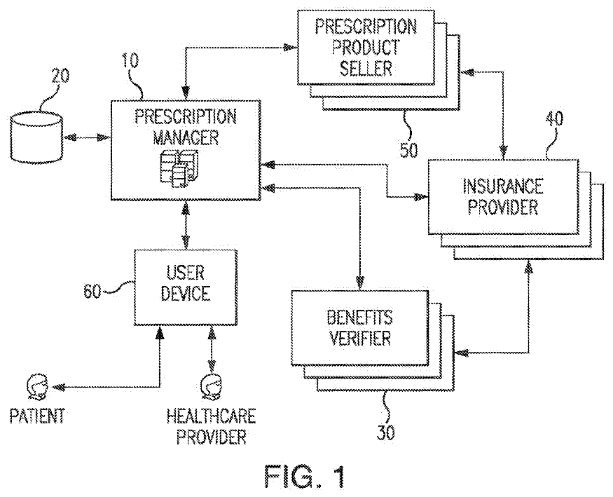

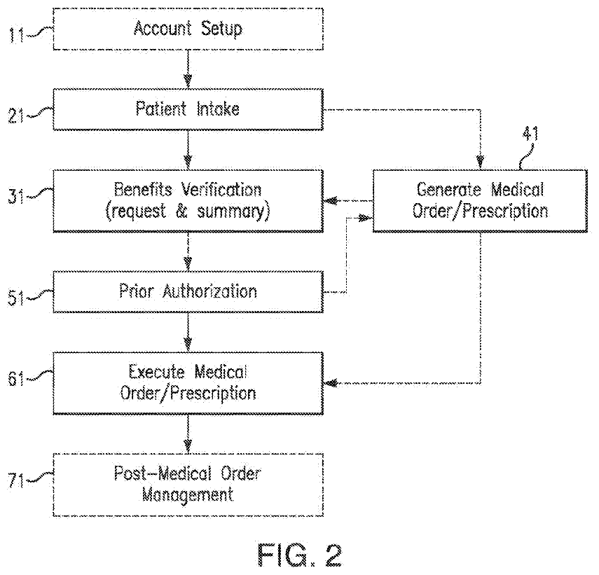

The accompanying figures, where like reference numerals refer to identical or functionally similar elements throughout the separate views, serve to further illustrate various embodiments and to explain various principles and advantages all in accordance with the disclosed subject matter. For purpose of explanation and illustration, and not limitation, exemplary embodiments of the method and system for facilitating a medical order/prescription of a prescription product for a patient covered by a provider in accordance with the disclosed subject matter are described below, with reference to FIG. 1 and FIG. 2. For purposes of clarity, the method and the system are described concurrently and in conjunction with each other, wherein reference numbers to the method illustrated in FIG. 2 will be made with parenthesis ( ), and reference to the system depicted FIG. 1 will be made without parenthesis.

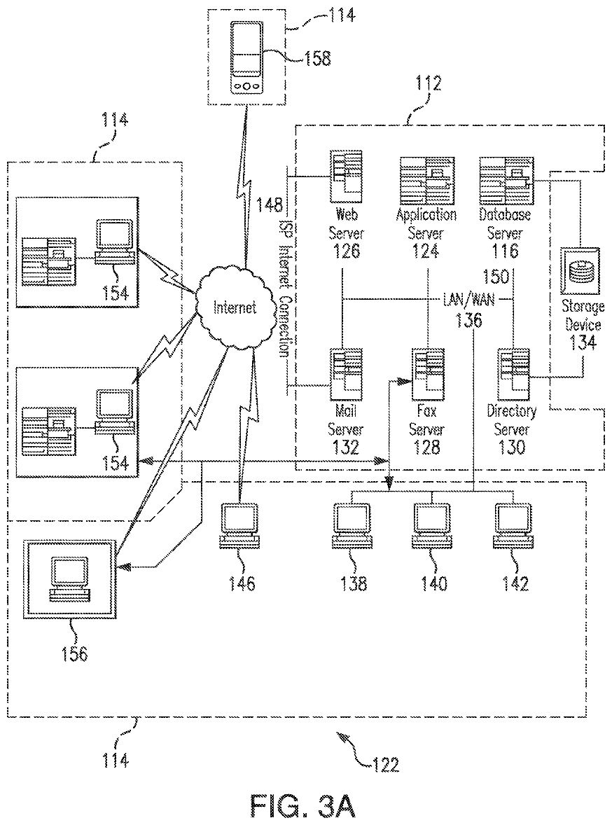

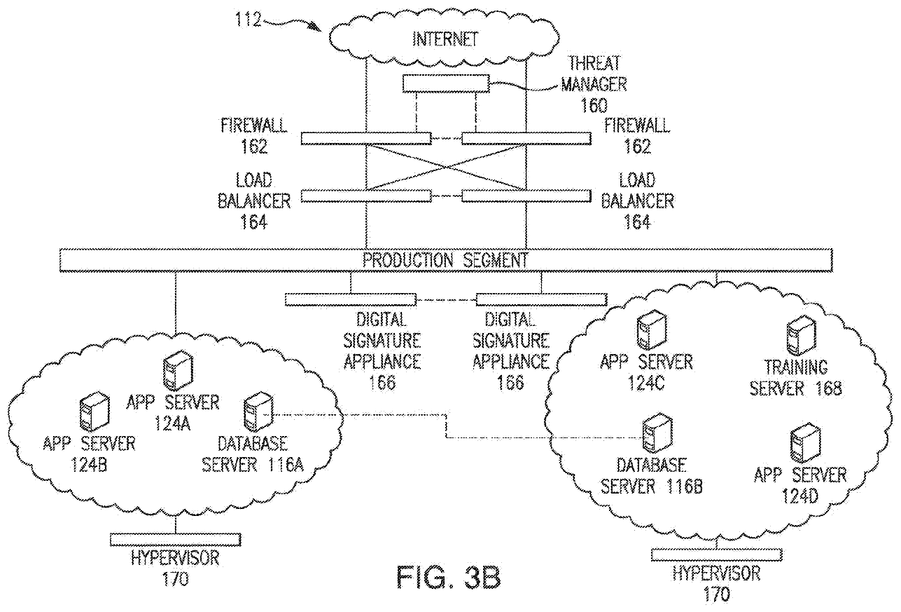

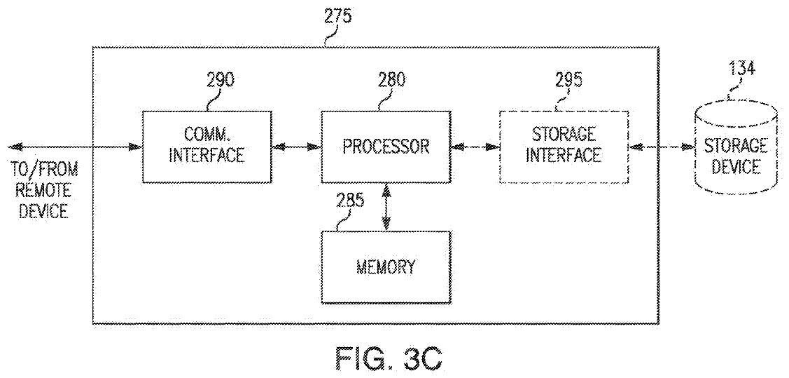

As embodied herein, for illustration, and with reference to FIG. 1 and FIG. 2, techniques for facilitating a medical order/prescription of a prescription product for a patient covered by a provider 40 can include the use of a “prescription manager” 10. The prescription manager 10 can include at least one memory device 20 and at least one processor. For example, the prescription manager 10 can be implemented on one or more computer systems (a stand alone computer, a server, a server cluster, a distributed computing system, a cloud based computing system, or the like), for example as depicted in FIG. 3 and discussed in more detail below.

In an exemplary embodiment, the prescription manager 10 can, for example, be implemented as a web-based software application hosting a corresponding website that includes a number of web pages (e.g., screens). One of ordinary skill in the art will appreciate that web-based software can transmit a user interface to a web-browser using, e.g., markup language such as HTML, XML, or the like, and can communicate with a web browser, e.g., using HTTPS, POST and/or GET requests. Moreover, one of ordinary skill in the art will appreciate that web-based software can be implemented as one or more web-services, and can employ REST, JSON, or the like. The software application can be stored on a non-transitory computer readable medium, such as a CD-ROM, DVD, Magnetic disk, ROM, RAM, or the like, the instructions of which can be read into a memory coupled to the one or more processors of the prescription manager 10. When executed, the software can instruct the processor to perform a particular function. As described herein below, for purposes of clarity, functionality of the prescription manager 10 may be described generally, without recitation that the processor of the prescription manager 10 is configured to perform the functionality. Alternatively, the prescription manager 10 can be implemented in hard-wired circuitry in place of, or in combination with, software instructions for implementation of the presently disclosed subject matter. Thus, embodiments of the presently disclosed subject matter are not limited to any specific combination of hardware and software, provided such hardware and software are configured to perform the method as disclosed herein.

As embodied herein, the prescription manager 10 facilitates a medical order or the prescription of a prescription product. That is, for example and as described in more detail below, the prescription manager 10 can facilitate the process of prescribing a prescription product to a patient by a healthcare provider, which can include facilitating benefits verification, prior authorization, and/or the generation and/or transmission of a medical order/prescription. Alternatively, the prescription manager 10 can facilitate the approval of payment for a prescription product, including, for example, facilitating prior authorization and/or facilitating approval of a reimbursement for a prescription product. As disclosed herein, a prescription product can include, but is not limited to, a medical product, a medical service, or the administration of a medical product. For example, the prescription product can be a medical product such as a drug, pharmaceutical, biologic, medical device. Additionally or alternatively, the prescription product can be a medical service, such as for example injection training, an eye examination, a spinal alignment, or the like. Additionally or alternatively, the prescription product can be the administration of a medical product, such as for example, the injection of a biologic at the office of a healthcare provider. As disclosed herein, a “medical order/prescription” can include either or both a prescription and a medical order, such as for example, the prescription of a controlled medication, or the medical order of a treatment or the like that need not require a prescription. For purposes of clarity, and not limitation, the description below is made primarily with reference to the process of a prescription. However, one of ordinary skill in the art will appreciate that the description regarding a prescription below can be equally applicable to a medical order. The system can be configured to facilitate only one particular prescription product and/or medical order, or allow for the selection of a prescription product and/or order from a number as stored in the system as further described.

The prescription manager 10 can manage predefined forms for the prescription product and/or service, for example as required by a plurality of providers 40. For example, the prescription manager 10 can maintain a list of authorization forms corresponding to a plurality of insurance providers 40. Additionally or alternatively, the prescription manager 10 can maintain a list of predefined forms used by different healthcare providers for different prescription products and/or services. Such predefined forms can be stored in electronic format (e.g., as Adobe Portable Document Format (PDF) files), in at least one memory device 20.

The prescription manager 10 can manage the acquisition of certain patient intake information (21) (e.g., with one or more suitably configured processors). The prescription manager 10 can include a receiver to receive certain information, such as prescription product information for the prescription product, patient intake information for the patient (including, for example, provider information), and a benefits verification summary. The prescription manager 10 can also include a transmitter for transmitting certain information, such as a benefits verification request. For example, in an exemplary embodiment, the prescription manager 10 can be connected to a network, such as the internet or an intranet, and the transmitter and receiver can include one or more network interface cards adapted to communicate via the network. In this manner, the transmitter and receiver can communicate with, for example, a user device 60, which can be operated by a healthcare provider and/or a patient. Additionally, the transmitter and receiver can communicate with one or more providers 40, prescription product sellers 50, and/or benefits verifiers 30. Additionally or alternatively, the transmitter and receiver and include input and output ports for communication with hardware adapted to provide data and/or receive and display data. For example, a keyboard and display device can be locally coupled to the prescription manager 10. As disclosed herein, the terms “transmit” and “receive” can include any means of electronic communications, including for example, TCP/IP, UDP, HTTP, facsimile, or the like. In like manner, the terms “transmitter” and “receiver” can include any device configured to transmit or receive electronic information, such as a network interface card (NIC), fax machine, or the like.

The prescription manager 10 can also manage the verification of benefits (including generation of a benefits verification request and acquisition of a benefits verification summary) for a patient (31) (e.g., with one or more suitably configured processors). The one or more processors of the prescription manager 10 can be configured to generate a benefits verification request for a patient based on at least patient intake information. For example, the benefits verification request can be generated based on biographical information, provider information, diagnosis information, and the like transmitted to (received by) the receiver (for example, by user device 60), as well as information from external sources which need not be received by the receiver. For example, certain patient intake information can be stored in the at least one memory device 20. Moreover, in an exemplary embodiment, the benefits verification request can be generated based on prescription product information for the prescription product, which can also be received by the receiver and/or stored in the at least one memory device 20. In an exemplary embodiment, the benefits verification request can be transmitted to a benefits verifier 30. The benefits verifier 30 can include any entity that can provide a summary of benefits a patient is entitled to for one or more patient providers 40. For example, the benefits verifier 30 can include a “pharmacy benefits manager” or “specialty pharmacy services provider,” (which can collectively be referred to herein as “pharmacy receiver”) which can independently generate a benefits verification summary for the patient. The benefits verification summary can be transmitted to (received by) the receiver of the prescription manager 10.

The prescription manager 10 can manage the selection, population, and release of certain predetermined forms, such as prior authorization forms, for a patient (51) (e.g., with one or more suitably configured processors). The one or more processors of the prescription manager 10 can be configured to select one of the predefined forms based on patient provider information (which can be included in the patient intake information), and populate at least one field of the selected predefined form based on the user intake information. For example, the processor can be configured to select the proper prior authorization form required by the patient's insurance provider and automatically populate fields that correspond to the patient intake information. In an exemplary embodiment, the prescription manager 10 can further be configured to transmit a request for additional patient information and receive additional patient information, for example to and from user device 60. For example, the additional patient information can include information required for the selected predefined form but not included in the patient intake information or the prescription product information.

The one or more processors can be configured to release the populated predefined form to facilitate the medical order/prescription of the prescription product to the patient. For example, the populated predefined form can be released to an insurance provider 40 of the patient. Alternatively, the populated predefined form can be released to the benefits verifier 30, which can in an exemplary embodiment further release the populated predefined form to an insurance provider 40 of the patient.

In an exemplary embodiment, the prescription manager 10 can manage the generating of (41) and transmission (61) of a prescription document or medical order document for the prescription product for the patient (e.g., with one or more suitably configured processors). For example, the one or more processors can be configured to receive a doctor's signature and generate a prescription document or medical order based on the prescription product information. Furthermore, the one or more processor can instruct the transmitter to transmit the generated prescription document to, for example, a prescription product seller 50. Moreover, in an exemplary embodiment, the prescription manager 10 can manage certain post-prescription processes (71), such as monitoring the status of a patient's prescription or providing the patient with certain additional or supplemental features, as described in more detail below.

Additional or alternative embodiments of the prescription manager 10 are described below, with reference to FIG. 3, for purposes of illustration, and not limitation.

With reference to FIG. 3A, an exemplary embodiment of the prescription manager, referred to herein as “server system” 112, can further include database server 116, an application or transaction server 124, a web server 126, a fax server 128, a directory server 130, and a mail server 132. A storage device 134 can be coupled to database server 116 and directory server 130. Servers 116, 124, 126, 128, 130, and 132 can be coupled in a local area network (LAN). a plurality of client sub-systems (also referred to as client systems 114) can be connected to server system 112. For example, the client sub-systems 114 can include a user device 60, which can be operated by a healthcare provider or a patient. Additionally, or alternatively, the client sub-systems 114 can include a computer device operated by a benefits verifier 30. In an exemplary embodiment, client systems 114 can be computers including a web browser, such that server system 112 is accessible to client systems 114 using the Internet or an intranet. In an exemplary embodiment, client systems 114 are tablet computing devices or any suitable mobile computing device, such as a tablet computer, a notebook computer, a netbook computer, a mobile phone, or the like.

Client systems 114 can be interconnected to the Internet through many interfaces including a network, such as a local area network (LAN) or a wide area network (WAN), dial-in-connections, cable modems, cellular networks, and special high-speed ISDN lines. Client systems 114 could be any device capable of interconnecting to the Internet including a web-based phone, personal digital assistant (PDA), tablet computer, or other web-based connectable equipment.

A database server 116 can be connected to memory device, storage device, or database (for example, storage device 134), which contains information on a variety of matters, as described below in greater detail. As embodied herein, for illustration, a centralized database is stored on server system 112 and can be accessed by logging onto server system 112 through one of client systems 114. In an alternative embodiment, a database is stored remotely from server system 112 and may be non-centralized. The database can store patient data, healthcare provider (HCP) data, health insurance company data, pharmacy data, forms, system usage data, audit trail data, and the like.

For purposes of illustration and not limitation, FIG. 3B depicts an exemplary server architecture for server system 112. Server system 112 can be connected to the Internet or other network through a collection of security appliances and/or software. In an exemplary embodiment, the collection can includes threat manager 160, a pair of firewall appliances 162A and 162B (collectively firewall appliances 162), and a pair of load balancers 164A and 164B (collectively load balancers 164). Threat manager 160 can provide vulnerability assessment and intrusion detection for server system 112. Threat manager 160 can be implemented in hardware, software, or a combination of hardware and software. Firewalls 162 generally permit or deny network transmissions based upon a set of rules to protect server system 112 from unauthorized access while permitting legitimate communications to pass. Firewalls 162 can be implemented in hardware, software, or a combination of hardware and software. Load balancers 164 can facilitate balancing traffic and sharing workload among components of system 112. Load balancers 164 can be implemented in hardware, software, or a combination of hardware and software.

A pair of digital signature appliances 166A and 166B (collectively digital signature appliances 166) can be connected on the protected side of server system 112. Digital signature appliances 166 can provide digital signature capture and security capabilities for the system disclosed herein. Digital signature appliances 166 can be implemented in hardware, software, or a combination of hardware and software. In the illustrated embodiment, server system 112 further includes four application servers 124A, 124B, 124C, and 124D (collectively servers 124), two database servers 116A and 116B (collectively servers 116), and a training server 168. Servers 116A, 124A, and 124B are servers virtualized by a first hypervisor 170A. Similarly, servers 116B, 124C, 124D, and 168 virtualized by a second hypervisor 170B. In other embodiments, servers 116, 124, and 170 are separate, physical, server machines.

FIG. 3C illustrates an exemplary configuration of a server computer device 275 such as server system 112 and prescription manager 10 (as shown in FIG. 1). Server computer device 275 can include, but is not limited to, database server 116, transaction server 124, web server 126, fax server 128, directory server 130, and mail server 132.

Server computer device 275 includes a processor 280 for executing instructions. Instructions can be stored in a memory area 285, for example. Processor 280 can include one or more processing units (e.g., in a multi-core configuration).

Processor 280 can be operatively coupled to a transmitter and receiver, i.e., a communication interface 290, such that server computer device 275 is capable of communicating with a remote device such as computer device 202 or another server computer device 275. For example, communication interface 290 can receive requests from client systems 114 via the Internet.

Processor 280 can also be operatively coupled to at least one memory, such as storage device 134. Storage device 134 can be any computer-operated hardware suitable for storing and/or retrieving data. In an exemplary embodiment, storage device 134 is integrated in server computer device 275. For example, server computer device 275 can include one or more hard disk drives as storage device 134. In other embodiments, storage device 134 is external to server computer device 275 and can be accessed by a plurality of server computer devices 275. For example, storage device 134 can include multiple storage units such as hard disks or solid state disks in a redundant array of inexpensive disks (RAID) configuration. Storage device 134 can include a storage area network (SAN) and/or a network attached storage (NAS) system.

In an exemplary embodiment, processor 280 can be operatively coupled to storage device 134 via a storage interface 295. Storage interface 295 can be any component capable of providing processor 280 with access to storage device 134. Storage interface 295 can include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 280 with access to storage device 134.

Memory areas 210 and 285 can include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are exemplary only, and are thus not limiting as to the types of memory usable for storage of a computer program.

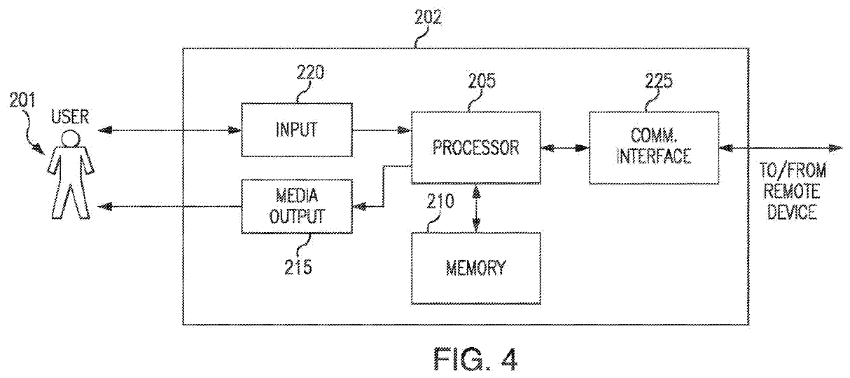

Additional or alternative embodiments of user device 60 are described below, with reference to FIG. 4, for purposes of illustration, and not limitation.

FIG. 4 illustrates an exemplary configuration of a user device 202 operated by user 201 such as client systems 114 (shown in FIG. 3) and user device 60 (shown in FIG. 1). User device 202 can be, for example, any device in communication with the prescription manager 10.

Computer device 202 can include a processor 205 for executing instructions. In an exemplary embodiment, executable instructions are stored in a memory 210. Processor 205 can include one or more processing units (e.g., in a multi-core configuration). Memory area 210 can be any device allowing information such as executable instructions and/or other data to be stored and retrieved. Memory area 210 may include one or more computer readable media.

Computer device 202 can also include at least one media output component 215 for presenting information to user 201. Media output component 215 can be any component capable of conveying information to user 201, such as a video adapter and/or an audio adapter. An output adapter can be operatively coupled to processor 205 and operatively couplable to an output device such as a display device (e.g., a liquid crystal display (LCD), organic light emitting diode (OLED) display, cathode ray tube (CRT), or “electronic ink” display) and/or an audio output device (e.g., a speaker or headphones).

In an exemplary embodiment, computer device 202 includes an input device 220 for receiving input from user 201, such as, for example, a keyboard, a scanner, a pointing device, a mouse, a stylus, a touch sensitive panel (e.g., a touch pad or a touch screen), a gyroscope, an accelerometer, a position detector, camera, or an audio input device. A single component such as a touch screen can function as both an output device of media output component 215 and input device 220. Moreover, computer device 202 can include more than one input device 220 for receiving input from user 201. For example, computer device can include the combination of a keyboard, a touch sensitive panel, and a scanner.

Computer device 202 can also include a communication interface 225, which is communicatively couplable to a remote device such as server system 112 (e.g., prescription manager 10). Communication interface 225 can include, for example, a wired or wireless network adapter or a wireless data transceiver for use with a mobile phone network (e.g., Global System for Mobile communications (GSM), Code Division Multiple Access (CDMA), 3G, 4G or Bluetooth) or other mobile data network (e.g., Worldwide Interoperability for Microwave Access (WIMAX)).

Stored in memory area 210 are, for example, computer readable instructions for providing a user interface to user 201 via media output component 215 and, optionally, receiving and processing input from input device 220. A user interface may include, among other possibilities, a web browser and client application. Web browsers enable users, such as user 201, to display and interact with media and other information typically embedded on a web page or a website from server system 112. A client application allows user 201 to interact with a server application from server system 112.

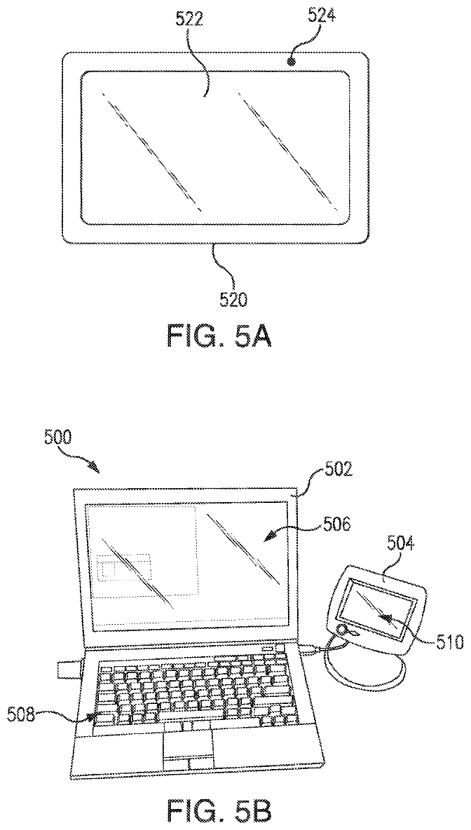

Additional or alternative embodiments of user device 50 are described below, with reference to FIGS. 5A and 5B, for purposes of illustration, and not limitation.

FIGS. 5A and 5B each depict an exemplary user device for use by a healthcare provider (“HCP”), operable as, for example, client systems 114 (shown in FIG. 3) and user device 60 (shown in FIG. 1). With reference to FIG. 5B, for purpose of illustration and as embodied herein, computing device 502 can include a display 506 and a keyboard 508. Computing device 502, also referred to herein as a remote input computer, includes a processor (not shown) for executing instructions. In an exemplary embodiment, executable instructions are stored in a memory area (not shown). For purpose of example, and not limitation, computing device 502, a tablet computing device, where display 506 is a touch screen display device operable to display images and data to a user and to receive input from the user via contact by the user (or an implement, such as a stylus, controlled by the user) with display 506. Rather than include an attached keyboard, the computing device 502 can include a virtual keyboard displayed on display device 506. In an exemplary embodiment, computing device 502 does not include an integrated physical keyboard, but is connectable, such as via mechanical connection, wireless connection, etc., to a physical keyboard. Moreover, in an exemplary embodiment, computing device 502 includes, or is attachable to a physical keyboard, and includes a virtual keyboard. Computing device 502 includes at least one communication interface (not shown), which is communicatively couplable to a remote device, such as server system 112. The communication interface may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)). Moreover, computing device 502 can include more than one communication interface, e.g. a wired network adapter and a wireless network adapter and/or a wireless data transceiver.

Additionally or alternatively, for purpose of illustration, user device (such as user device, for example, 7340) can include a computer 502 and a scanner 504 coupled together. The computer, such as a notebook computer 502, includes a display 506 and a keyboard 508. As disclosed herein, for illustration, display 506 may be a touch-sensitive device (e.g., a touch screen) that is capable of detecting input from a user (e.g., the healthcare provider) when the user touches display 506 with, for example, a finger or a stylus. For example, the user can single or double tap on a user-interface component on touch-sensitive display 506 to select or activate that component. The user may pinch open or pinch close a user-interface component on touch-sensitive display 506 with two fingers to zoom in or zoom out or open or close that component. The user can swipe across touch-sensitive display 506 (e.g., swiping left, right, up, or down) to view a series of user-interface components. As disclosed herein, for illustration, the user can sign his/her name on touch-sensitive display 506 (e.g., with a stylus or a finger), and the user's signature can be captured and stored in electronic format (e.g., as an image). In addition, the user can provide input to notebook computer 502 using keyboard 508 (e.g., typing characters).

As disclosed herein, for illustration, a web browser can be installed and executed on notebook computer 502. The healthcare provider can access prescription manager 7310 using the web browser. In this case, prescription manager 7310 can implement a web-based application (e.g., a website including a number of web pages), and the healthcare provider may access the website corresponding to prescription manager 7310 by inputting the correct uniform resource locator (URL) for the website in the web browser. Information transmitted between notebook computer 502 and prescription manager 7310 can be encrypted and sent over a secure network connection in order to protect, for example, patient privacy.

For example, with reference to FIG. 5B, HCP system 500 can include a computing device 502 and a scanning device 504. Scanning device 504 can be communicatively coupled to computing device 502 to transmit data (e.g., images) to computing device 502. Scanning device 504 can be operable to scan, or image, items placed in proximity to a scanning window 510. In an exemplary embodiment, scanning device 504 and/or user device SOO can be configured (either alone or collectively), such as via instructions stored in a memory device, to perform OCR on scanned images and transmit the recognized characters to computing device 502. As described herein, scanning device 504 can be used to scan a patient identification document, such as for example a license, medical insurance card, prescription benefits card, or the like.

In an exemplary embodiment, with reference to FIG. 5A, computing device 502 is a tablet computing device, optionally including a camera 524. In an exemplary embodiment, display 522 is a touch screen display device operable to display images and data to a user and to receive input from the user via contact by the user (or an implement, such as a stylus, controlled by the user) with display 522. Alternatively, for purpose of illustration, tablet 520 can be coupled to a card scanner (e.g., scanner 504 in FIG. 5B). Touch-sensitive display 522 is capable of detecting input from a user (e.g., the healthcare provider) when the user touches display 522 with, for example, a finger or a stylus. As disclosed herein, for illustration, the user can sign his/her name on touch-sensitive display 522 (e.g., with a stylus or a finger), and the user's signature can be captured and stored in electronic format (e.g., as an image). In addition or alternatively, a pre-captured signature can be retrieved from a data store. For purpose of illustration, camera 524 can be used to take digital photos of an object, such as a patient's identification card, driver's license, or insurance card(s). For example, the patient or the healthcare provider can hold a card in front of camera 524 and push the control button. Alternatively, for purpose of illustration, the card scanner coupled to tablet 520 can be used to scan the card, as described above in connection with FIG. 5B. OCR or image recognition techniques, implemented as software executing on tablet 520, can help extract information provided on the card, convert the information to electronic format, and store the information in individual fields.

In an exemplary embodiment, computing device 502 is configured to capture an electronic signature. Computing device 502 is configured to display a signature block on display 506 when capture of an electronic signature is desired. The user, e.g., the HCP, can sign his or her signature in the signature block on the display 506 utilizing a touch screen stylus. In an exemplary embodiment, the user's signature can be captured and stored in electronic format (e.g., as an image). Additionally, as embodied herein, for example in jurisdictions that disallow the use of electronic signatures, the HCP can sign his or her signature on a printed form using a visible medium writing implement such as an ink pen, a pencil, a marker, or the like. In other embodiments, the HCP can align a printed form with display 506 and sign his or her signature on the printed form using a special writing device that includes both a visible medium writing implement as well as an electronic writing implement, such that an electronic signature is captured by the system in addition to the physical signature. In such embodiments, a visible, physical, handwritten signature results on the printed form and computing device 502 captures a digital representation of the physical, handwritten signature as an electronic signature substantially simultaneously. In an exemplary embodiment, computing device 502 displays registration marks indicating to the user how to align the paper form against display 506. Furthermore, scanning device 504 can be configured, additionally or alternatively, to capture an electronic signature in a manner similar to computing device 502. Moreover, a user device system 500 operated by an HCP can include a separate signature capture device (not shown) operable as described herein to capture a digital representation of a handwritten signature. In another embodiment, the HCP can utilize a scanning device or digital capture device, such as a digital camera, to capture an image of their physical signature. The captured image can then be transmitted to the system through various transmission means.

Computing device 502 can include a user interface to permit computing device 502, and HCP user device system 500 in general, to function in accordance with the medical treatment coordination systems and methods described herein. The user interface can be stored in a memory device and/or can be stored remotely (such as on server system 112) and accessed by computing device 502, such as via a web browser. Moreover, the exemplary computing device 502 can store data input to the user interface in a memory device of computing device 502, and/or can store the input data remotely, such as in a database.

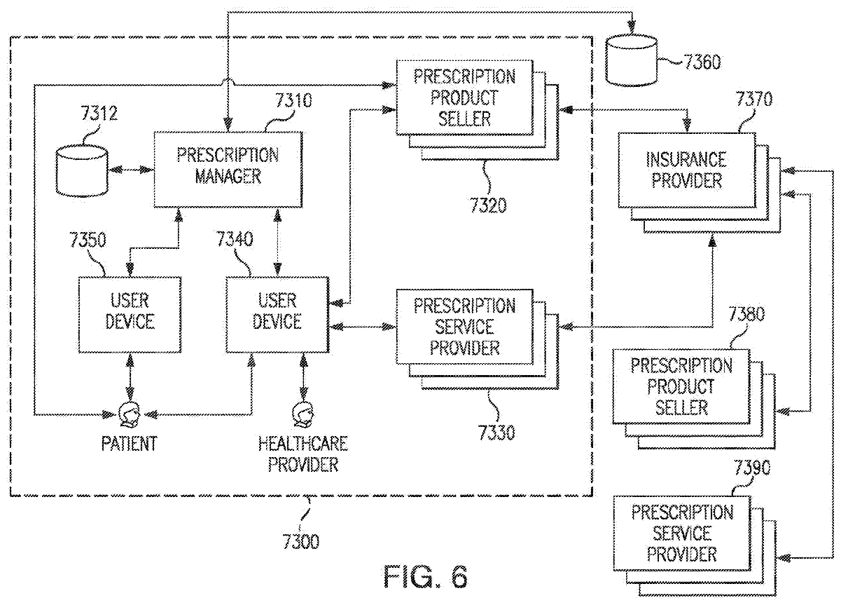

Additional or alternative embodiments of the techniques described above are described in detail below, with reference to FIG. 6, for purposes of illustration, and not limitation.

FIG. 6 illustrates an exemplary system 7300 that is operable to facilitate healthcare providers and their patients obtain insurance authorizations for products or services the healthcare providers prescribe to their patients. In an exemplary embodiment, system 7300 can include a prescription manager 7310 for managing authorization forms for prescription products and/or services required by insurance providers and for helping healthcare providers fill the necessary forms in order to obtain authorizations for prescription products and/or services from insurance providers. For example, and not limitation, prescription manager 7310 can maintain a list of authorization forms required by different insurance providers or used by different healthcare providers for different prescription products and/or services. These authorization forms can be updated as needed (e.g., new forms can be added; existing forms can be modified; expired forms can be deleted; or the like). For example, an insurance authorization form in paper format can be scanned (e.g., using a document scanner), converted to a fill-able form (e.g., using an optical character recognition (OCR) program), and stored. Additionally or alternatively, system 7300, and more specifically, prescription manager 7310, can be communicatively or electronically linked with various insurance providers, and the insurance providers can electronically upload and update respective forms into system 7300. When needed, prescription manager 7310 can select appropriate forms required by specific insurance providers for specific prescription products and/or services, fill the fields in the selected forms based on information available to or obtained by prescription manager 7310, and send the completed forms to healthcare providers for review and signatures.

In addition, for purpose of illustration, prescription manager 7310 can help a healthcare provider manage prescriptions of his/her patients. For example, prescription manager 7310 can send reminders to the healthcare provider if the healthcare provider does not review and sign completed insurance authorization forms after receiving them from prescription manager 7310 for some period of time. Prescription manager 7310 can notify the healthcare provider when specific prescriptions have been filled. For purpose of illustration, prescription manager 7310 can help a patient use his/her prescriptions. For example, prescription manager 7310 can provide instructions (e.g., videos or audio instructions) on how to take a prescription medication, frequently asked questions (FAQ) and answers relating to possible side effects of the prescription medication, or the like. Additionally, prescription manager 7310 can provide notifications to the patient indicating status of his/her prescription (e.g., if the patient's prescription is ready to be picked up or shipped, if the patient's prescription has been denied, or if the healthcare provider has not completed the prescription/authorization process)

As disclosed herein, for illustration, prescription manager 7310 can implement a user interface so that its users can access various functions provided by prescription manager 7310 with relative ease. The user interface can include any number of screens. For purpose of illustration, the user interface can be a web-based user interface, implemented as a web-based software application hosting a corresponding website that includes a number of web pages (i.e., screens). For example, a healthcare provider or patient can access the corresponding website using a web browser executing on a user device.

Additionally, prescription manager 7310 can be implemented on one or more computer systems (e.g., servers), as described in more detail above. FIG. 3 (described in more detail above) illustrates a exemplary servers, which can be used to implement prescription manager 7310. The operations or functions performed by prescription manager 7310 can be implemented as computer software, which can be stored in non-transitory computer-readable medium and executed on the computer systems. As disclosed herein, for illustration, prescription manager 7310 can have various types of users (e.g., doctors, nurses, office staff, patients, pharmacists, or the like). Some of the functions performed by prescription manager 7310 can be commonly applicable to all types of users, while other functions can be applicable to specific types of users (e.g., functions in connection with proscribing a product or service can be applicable specifically to doctors).

As disclosed herein, for illustration, prescription manager 7310 can include or be communicatively linked to one or more data stores 7312 so that information stored in each data store 7312 is accessible to prescription manager 7310. Data stores 7312 can be used to store any applicable information. For example, as described above with reference to FIG. 1, the insurance authorization forms can be stored in data stores 7312 in electronic format (e.g., as PDF, text, extensible markup language (XML), binary data, comma separated data, or any other applicable electronic formats). Other information, such as information concerning patients (e.g., patient records, such as names, addresses, medical histories, insurance providers, or the like of the patients), healthcare providers (e.g., names, practice fields, specializations, hospital or medial facility affiliations, or the like of the healthcare providers), or prescription products or services (e.g., recommended dosages, possible side effects, treatment procedures, manufactures, or the like of the prescription products) can also be stored in data stores 7312. A data store 7312 can be any applicable type of storage device, such as internal or external or network drives. As disclosed herein, for illustration, data store 7312 can further include an electronic medical record system (EMR). The EMR can contain patient data, such as medical records, test results, or the like, and such data can be shared with prescription manager 7310 as contemplated herein.

As disclosed herein, for illustration, a user device 7340 can be associated with a healthcare provider. The healthcare provider can access prescription manager 7310 via user device 7340. In addition, while a patient is visiting the healthcare provider, the patient can also access prescription manager 7310 via user device 7340, with consent from the healthcare provider. For example, the healthcare provider or the patient can send patient information to prescription manager 7310 using user device 7340. The healthcare provider can prescribe a specific product or service to the patient and communicate the prescription to prescription manager 7310 using user device 7340. If an insurance authorization form is needed for the prescription product or service, then prescription manager 7310 can send a completed insurance authorization form for the prescription product or service to user device 7340 so that the healthcare provider can review and sign the form. For purpose of illustration, the healthcare provider can have an account with prescription manager 7310. Information relevant to the healthcare provider (e.g., patients, prescriptions, pending insurance authorization forms, reminders, or the like) can be included in the healthcare provider's account. The healthcare provider can log into his/her account with prescription manager 7310 to review available information and perform other related activities.

For purpose of illustration, and not limitation, user device 7340 can be a mobile device, such as a tablet or notebook computer or a smart telephone, and can include various sensors. User device 7340 can communicate with prescription manager 7310 over a computer or communication network via a wireless connection to the network (e.g., using the WiFi or 3G or 4G connection available at the office of the healthcare provider). Information transmitted between user device 7340 and prescription manager 7310 can be encrypted (e.g., to protect patient privacy) and optionally compressed (e.g., to reduce data size). As disclosed herein, for illustration, user device 7340 is capable of sending electronic mails (emails), texts, faxes, and/or electronic data to specific email addresses, fax numbers, and/or data stores (e.g., data store 7312). In the case of sending electronic faxes, user device 7340 can be connected to a telephone line. There can be a fax software application installed and executed on user device 7340 for sending faxes to specific fax numbers over the telephone line. Alternatively, electronic faxes can be sent over a computer network, in which case the telephone line is not required.

As disclosed herein, for illustration, and as noted above, the prescription manager can be implemented as a web-based application, and a user device 7340 can include a web browser for accessing the prescription manager and displaying the user interface. Additionally, as noted above, scanner 504 can be a card scanner capable of scanning various types of cards, such as a patient's identification card, driver's license, or insurance card. Scanner 504 can capture information on such cards (e.g., the patient's name, address, birth date, gender, driver's license or identification number, insurance provider, insurance number, or the like). For purpose of illustration, the information captured from scanning the patient's cards can be stored in individual fields, where each field can have a field name and a field value. For example, for patient “John Smith”, there can be a field for his name, where the field name is “patient name” and the field value is “John Smith”. There can be a second field for his birth date, where the field name is “patient birth date” and the field value is “15 Jun. 1971”. There can be a third field for his insurance provider, where the field name is “insurance provider” and the field value is “Blue Shield of California”. There can be a fourth field for his insurance number, where the field name is “insurance number” and the field value is “54917850”.

Furthermore, the data representing each piece of information captured by user device 7340 as described above can be tagged with a unique identifier. For example, the patient's first name can be tagged with “F-Name” as its identifier, and the patient's last name can be tagged with “L-Name” as its identifier. As insurance authorization forms are added to system 7300, fields within the forms can be tagged with these unique identifiers as well. Therefore, as the data are captured (e.g., by user device 7340) or retrieved from a data store (e.g., from data store 7312), the tagged fields within each authorization form can be automatically populated with the necessary data. For purpose of illustration, system 7300 can include a technical user interface, where newly uploaded forms can be edited (e.g., by a system administrator or system user) to include data tags, thereby adding the ability to keep system 7300 and the insurance authorization forms up to date.

As embodied herein, OCR technique can be used to extract information from scanned images of the cards. There can be software implementing OCR functions. In some cases, the OCR software can be part of and executed on notebook computer 502. In other cases, the OCR software can be part of and executed on scanner 504. In addition, the patient or the healthcare provider can review the scanned information and manually input or correct individual field values if necessary (e.g., typing information into notebook computer 502 using keyboard 508).

Furthermore, a user device 7350 can be associated with a patient. The patient can access prescription manager 7310 via user device 7350. For purpose of illustration, the patient can have an account with prescription manager 7310. The patient can log into his/her account using user device 7350 and review information concerning his/her prescription products or services. For example, the patient can access the website corresponding to prescription manager 7310 using a web browser installed and executing on user device 7350.

User device 7350 can be a mobile device, such as a tablet or notebook computer or smart telephone, or a stationary device, such as a desk computer. User device 7350 can communicate with prescription manager 7310 over a computer or communication network via a wireless (e.g., WiFi, 3G, 4G) or wired (e.g., Ethernet) connection to the network. Information transmitted between user device 7350 and prescription manager 7310 can be encrypted (e.g., to protect patient privacy) and optionally compressed (e.g., to reduce data size).

As embodied here, system 7300 can include one or more prescription product sellers 7320 (e.g., a pharmacy for selling prescription medications). In addition or alternatively, As disclosed herein, for illustration, system 7300 can include one or more prescription service providers 7330 (e.g., a specialist for providing healthcare services in a specific field, such as a cardiologist or a brain surgeon). The healthcare provider can communicate with prescription product sellers 7320 and/or prescription service providers 7330, as appropriate, via user device 7340. For example, the healthcare provider can send electronic mails (emails) or faxes to prescription product sellers 7320 or prescription service providers 7330. The prescription product seller 7320 or prescription service provider 7330 can be associated with a user device (not shown), such as a computing device connected to a network, for accessing the Internet and optionally for communicating with other entities (e.g., sending and receiving emails).

As disclosed herein, for illustration, there can be one or more data stores 7360 for storing patient records (e.g., electronic medical records system). Data stores 7360 can or can not be part of system 7300. For example, a data store 7360 can be associated with a prescription service provider 7330, in which case it can be part of system 7300. Alternatively, a data store 7360 can be associated with an independent third party (e.g., a hospital), in which case it can not be part of system 7300. In some cases, prescription manager 7310 can be able to access data stores 7360 to retrieve information (e.g., medical records) of the patient.

As disclosed herein, for illustration, the patient can have one or more insurance providers 7370. In some case, a patient can have just one insurance provider 7370. In other cases, a patient can have multiple instance providers 7370 (e.g., a primary provider and one or more secondary or supplemental providers). Prescription product sellers 7320 or prescription service providers 7330 can communicate with each insurance provider 7370 of the patient through any applicable means (e.g., telephone, fax, email, or the like) to obtain authorization from each insurance provider 7370 for products or services prescribed to the patient (e.g., by the healthcare provider).

Sometimes, an insurance provider 7370 can have one or more designated prescription product seller(s) 7380 and/or prescription service provider(s) 7390, which are not part of system 7300. In this case, the patient is required to obtain the prescribed product or service from prescription product seller(s) 7380 or prescription service provider(s) 7390 associated with insurance provider 7370 in order for insurance provider 7370 to agree to pay for the prescribed product or service.

As disclosed herein, for illustration, whenever applicable, system 7300 (e.g., its operations and functionalities) complies with the requirements from health Insurance Portability and Accountability Act (HIPAA). For example, if certain type of information should not be accessible to a specific party (e.g., a prescription product manufacturer or service provider) according to HIPAA requirements or other confidentiality concerns, system 7300 can implement information-control or information-protection measures that ensure the specific party cannot access that type of information. As another example, to protect patient privacy, information transmitted over a computer or communication network (e.g., the Internet), such as information transmitted between prescription manager 7310 and user device 7340 or 7350, can be encrypted.

As disclosed herein, for illustration, in order to use system 7300, a healthcare provider is required to first register and establish a user account with prescription manager 7310 (e.g., at the corresponding website). Once an account has been established, information concerning the healthcare provider can be stored with prescription manager 7310 in the healthcare provider's account. For purpose of illustration, a user account of a healthcare provider can be identified with a unique user name and protected by a password, which can be used to log into the account. In addition, a user account of a healthcare provider can have any number of authorized users. As an example, an account established for a doctor can have the doctor as one of its users. It can also have nurses or office staff working for the doctor as its other authorized users. The nurses or office staff can log into the account and perform various actions with the permission and under the supervision of the doctor. As another example, multiple doctors and their staff members sharing the same clinic can establish and share a single user account. For purpose of illustration, there can be a designated user (e.g., an account administrator) who is responsible for managing the account. The administrator can be able to modify the information associated with the account.

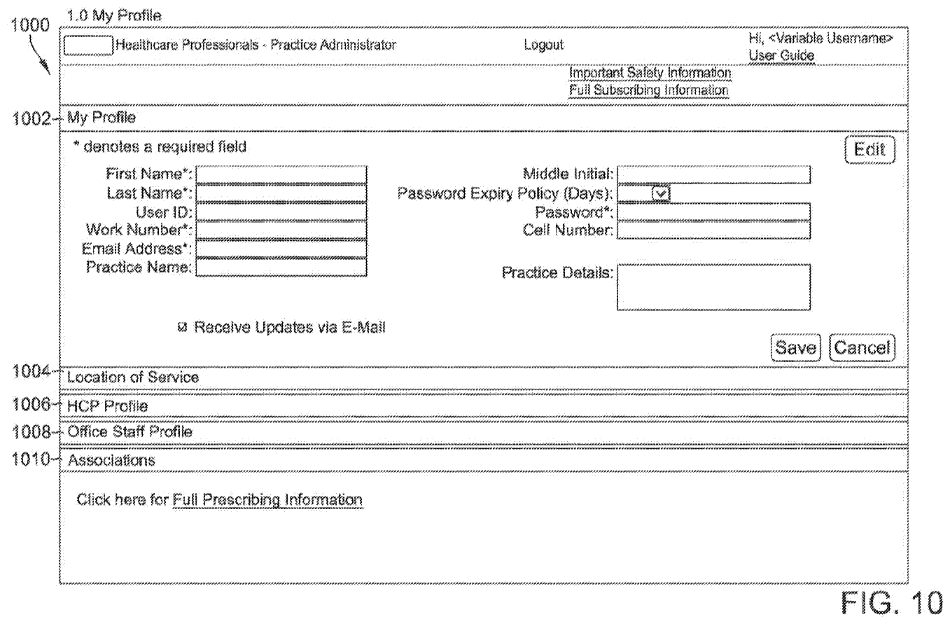

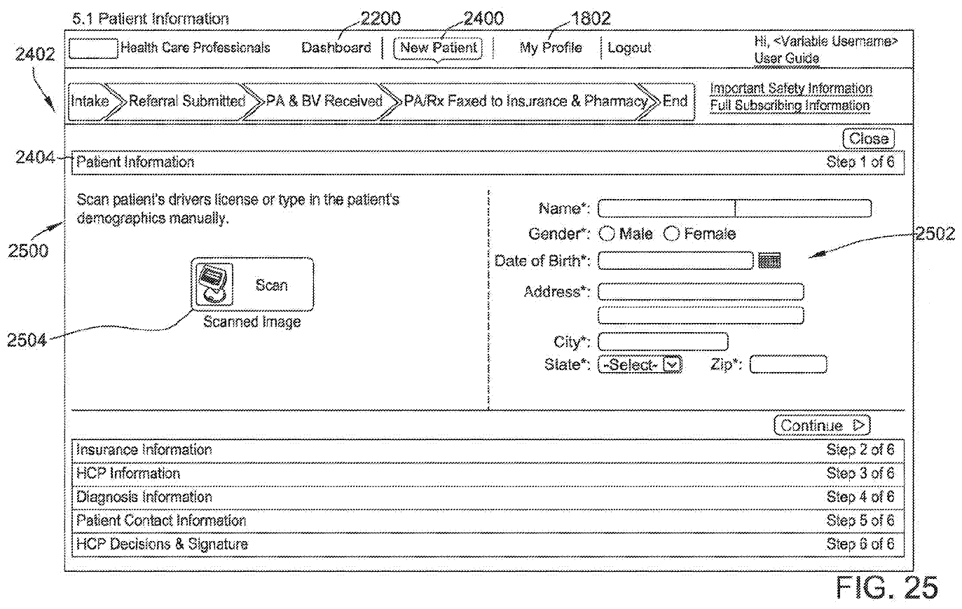

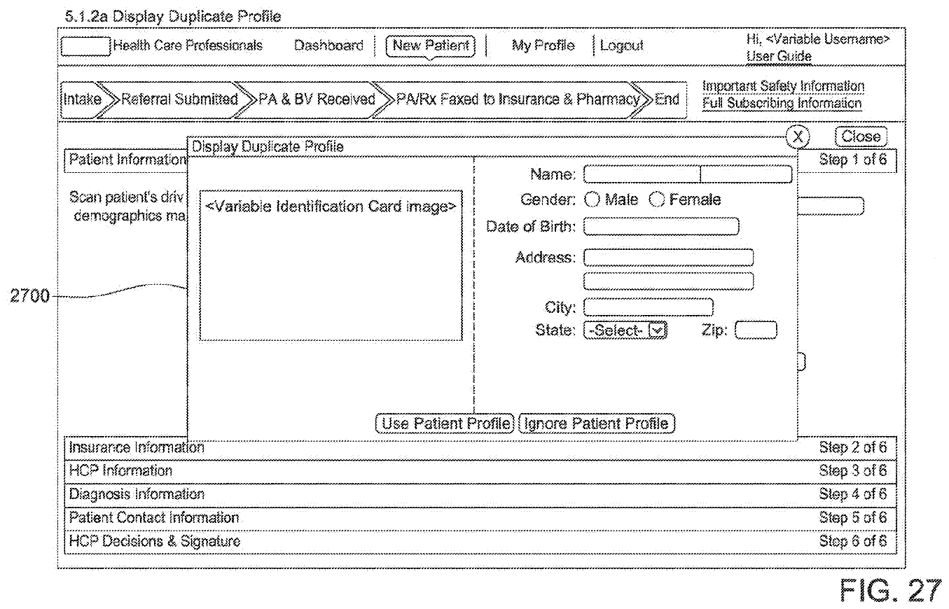

In accordance with another aspect of the disclosed subject matter, the user interface provided by prescription manager 7310 can include a series of screens (e.g., web pages), accessible with a user device (e.g., user device 7340) associated with a healthcare provider, to guide the healthcare provider or the designated account administrator through the account registration process. At various screens, the healthcare provider can input various types of information to be saved with prescription manager 7310 in the healthcare provider's account. For example, FIGS. 10-20 illustrate a representative series of screens for guiding a healthcare provider to register and establish a user account (11) with prescription manager 7310, which can include, for example, entering information about the HCP, such as names, affiliations, locations, staff, and electronic signature information. Additionally, FIGS. 25-30 illustrate example screens to guide a healthcare provider to scan a patient's cards in order to extract the necessary patient information automatically when performing the intake process (21) for a patient (e.g., using user device 7340 that includes a card scanner). Additionally, as described in more detail below, benefits verification (31) and prior authorization (51) can be performed, and a medical order/prescription can be generated (41) and transmitted (61) via a series of screens.

For purpose of illustration and not limitation, detailed description will now be made of various additional and alternative embodiments of the method for facilitating medical order and/or prescription of a prescription product disclosed herein. As noted above, the prescription manager can facilitate the medical order/prescription of a prescription product for a patient, which can include setting up user accounts (11), such as for the patient, HCP, and/or other third parties such as a benefits verifier 30. Patient intake information can be received (21), benefits verification (31) and prior authorization (51) can be performed, and a medical order/prescription can be generated (41) and transmitted (61).

As noted above, prescription manager (including, for example and without limitation, various embodiments of the prescription manager, depicted in the figures as 10, 7310, and 112) can manage account information for a variety of users of the system (11), either alone or in combination with one or more user devices (including, for example and without limitation, various embodiments of the user devices, depicted in the figures as 60, 500, 522, and 114). Different users of the system can certain categories of accounts. For example, an HCP can have an HCP account, and administrator can have an administrator account, patients can have patient accounts, and certain benefits verifiers (such as, for example, a pharmacy receiver) can have an account. In this manner, each party can access the systems disclosed herein through, for example, the one or more user devices described herein.

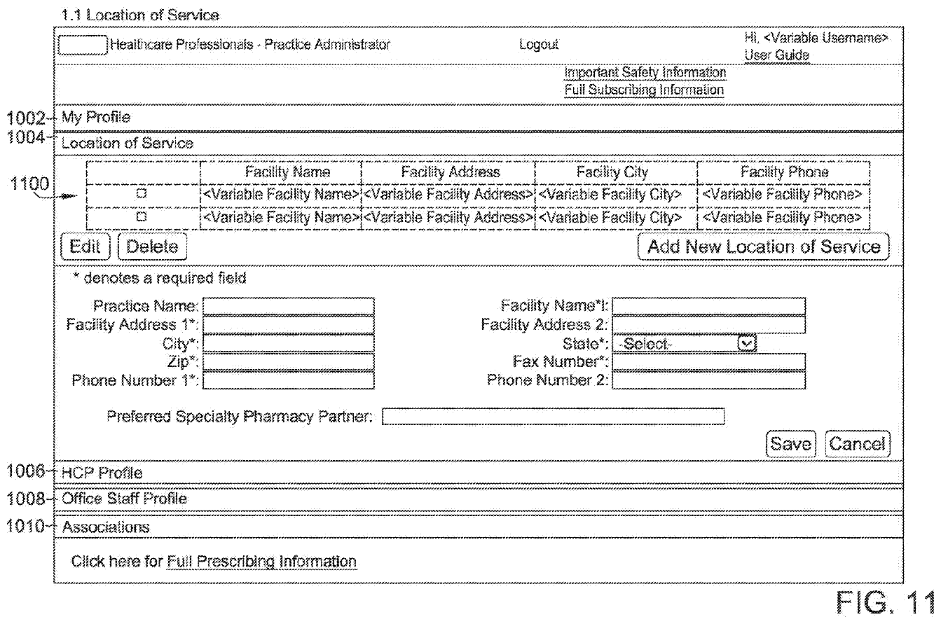

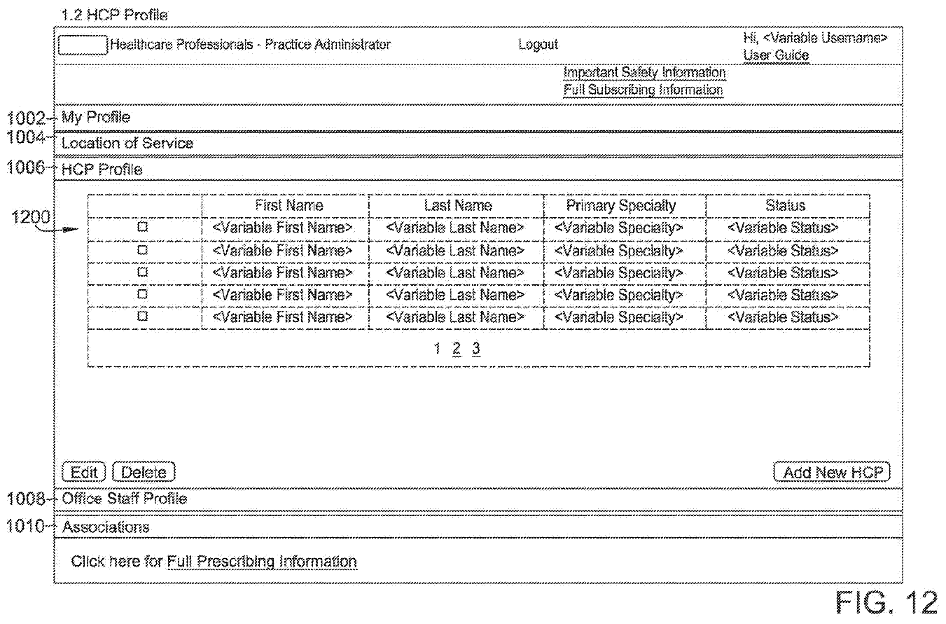

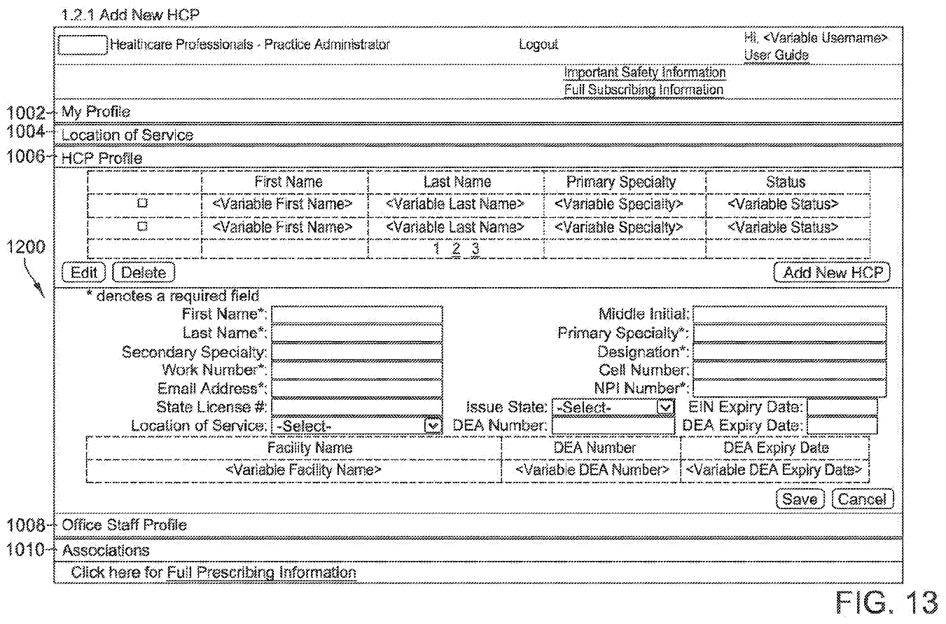

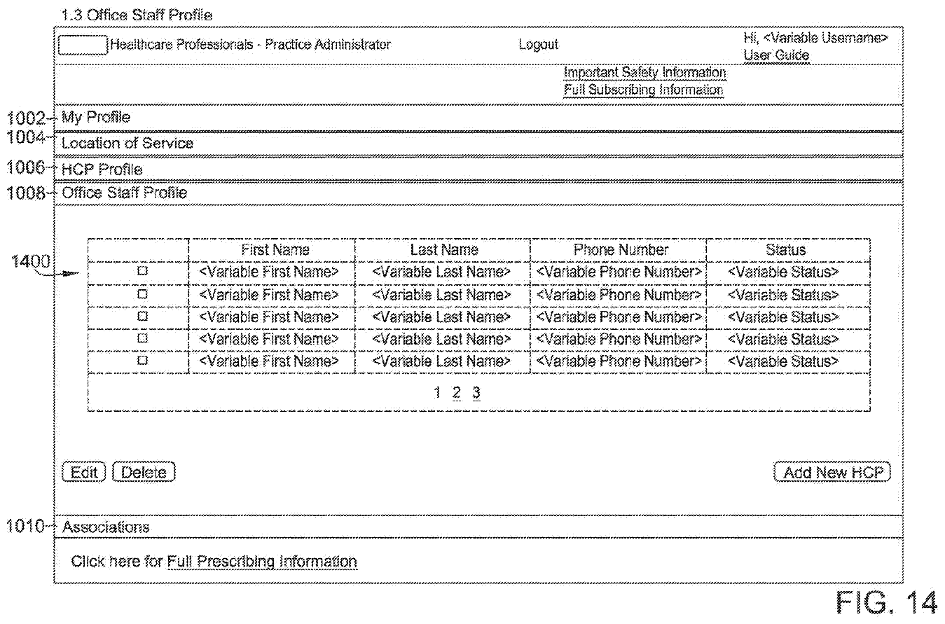

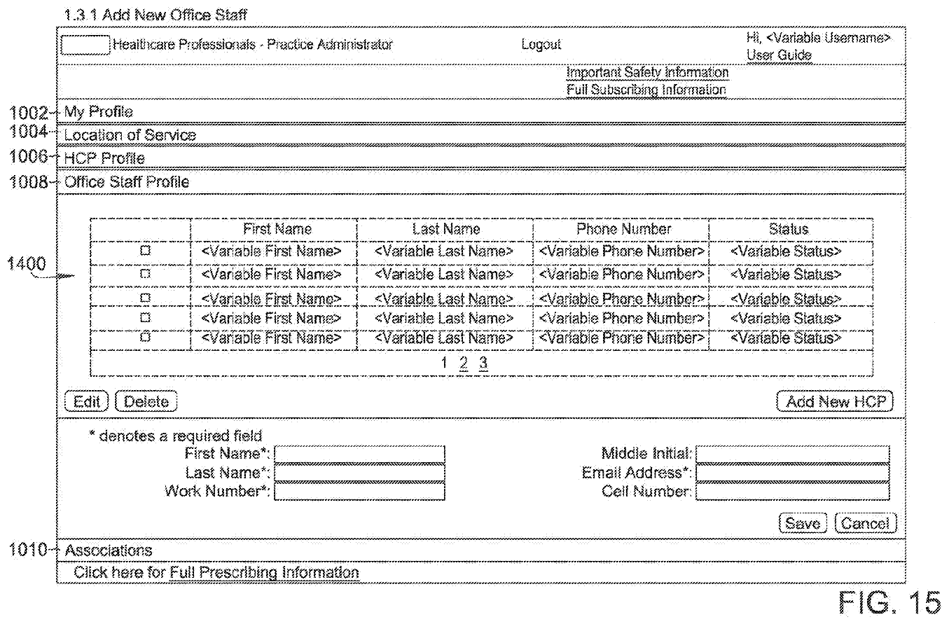

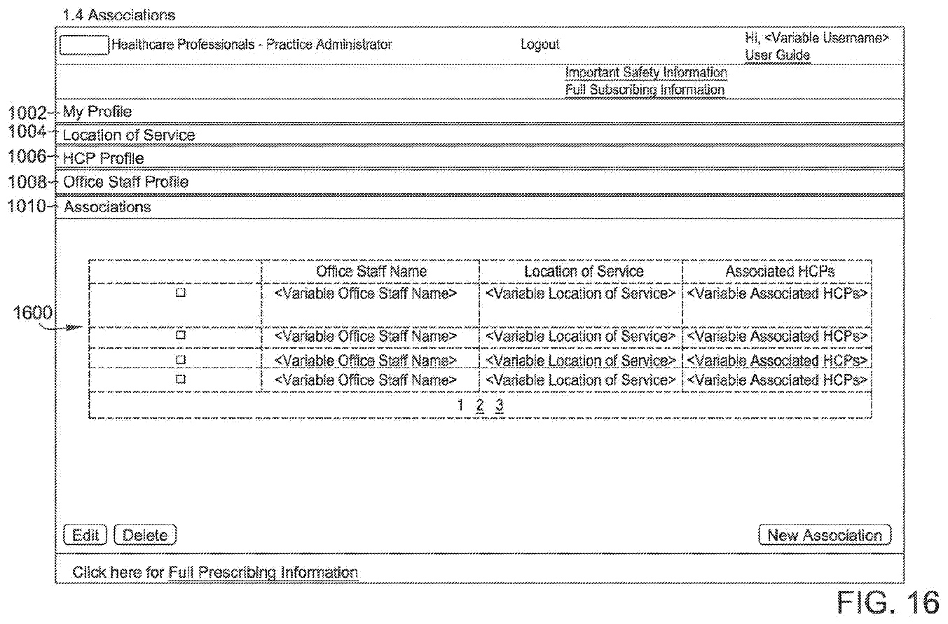

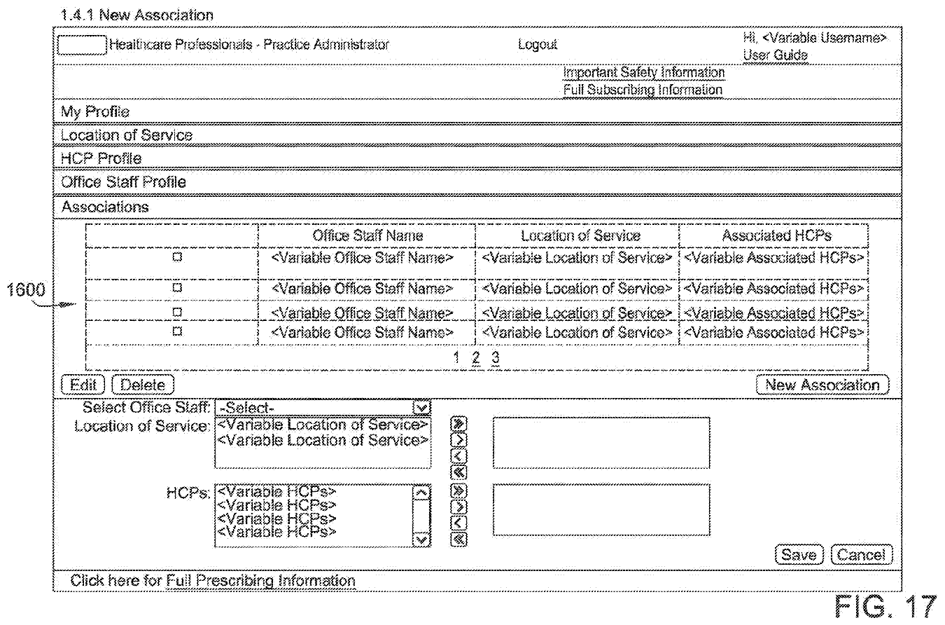

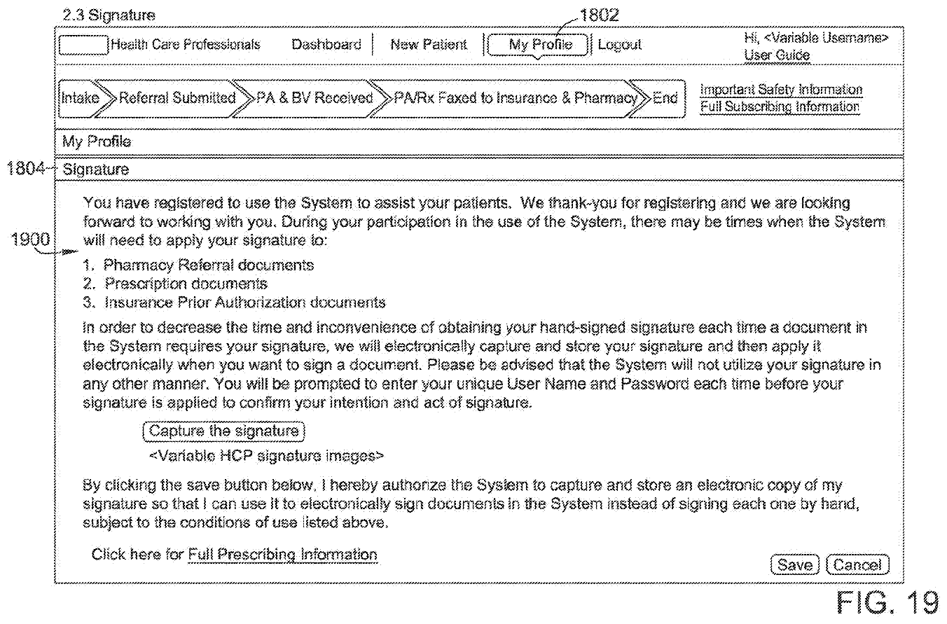

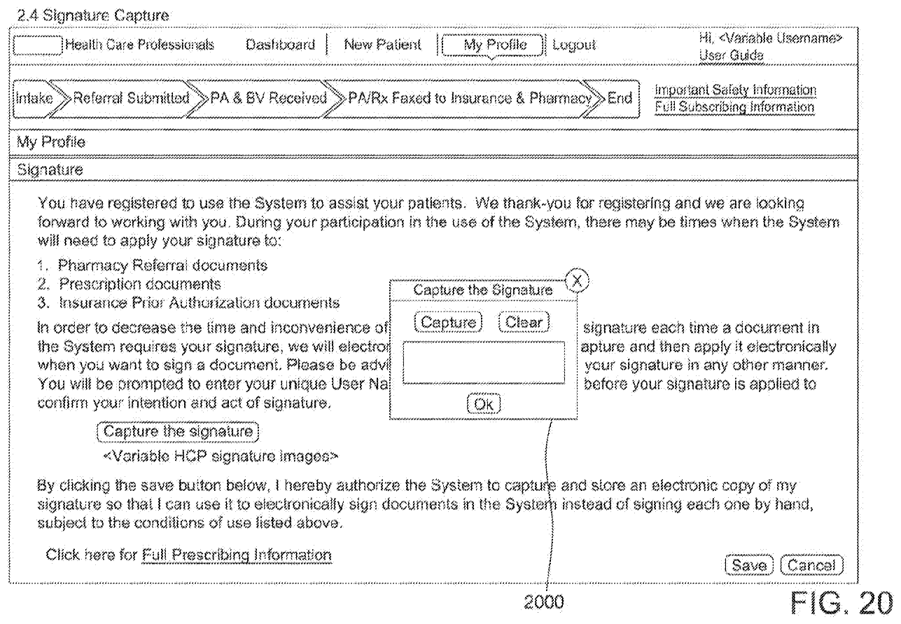

FIGS. 10-20 illustrate an example series of screens for guiding a healthcare provider to register and establish a user account (11) with prescription manager 7310, which can include, for example, entering information about the HCP, such as names, affiliations, locations, staff, and electronic signature information. For purpose of illustration, information in a healthcare provider's account can be organized into categories and displayed based on its relatedness. For example, from “My Profile” tab 1002 illustrated in FIG. 10, a healthcare provider can enter his/her name, user name, password, and contact information (e.g., telephone numbers). Alternatively, the administrator of the account can enter his/her information through tab 1002. From “Location of Service” tab 1100 illustrated in FIG. 11, the healthcare provider can enter the facilities with which he/she is affiliated, or his/her office location. From “HCP Profile” tab 1200 illustrated in FIGS. 12 and 13, the authorized users of the account, who are healthcare providers (e.g., doctors, nurses), can be displayed and entered. From “Office Staff Profile” tab 1400 illustrated in FIGS. 14 and 15, the authorized users of the account, who are staff members, can be displayed and entered. From “Associations” tab 1600 illustrated in FIGS. 16 and 17, associations of the healthcare provider can be displayed and entered. From “Signature” tab 1804 illustrated in FIGS. 19 and 20, the healthcare provider can have an electronic signature stored with his/her account or on user device 7340. To do so, the healthcare provider can sign his/her name using, for example, a stylus on the touch-sensitive screen of user device 7340.

As disclosed herein, for illustration, in order to use system 7300, a patient can be required to first register and establish a user account (11) with prescription manager 7310 (e.g., at the corresponding website). Once an account has been established, information concerning the patient can be stored with prescription manager 7310 in the patient's account. For purpose of illustration, a user account of a patient can be identified with a unique user name and protected by a password.

A patient can register a user account with prescription manager 7310 on his/her own (e.g., using user device 7350 associated with the patient), or can do so when visiting a healthcare provider (e.g., at the healthcare provider's office, using user device 7340 associated with the healthcare provider). For example, when the patient is visiting the healthcare provider and the healthcare provider decides to prescribe a product or service to the patient that requires insurance authorization, if the patient does not already have a user account with prescription manager 7310 and if the patient agrees, the healthcare provider can initiate an intake process (21) for the patient at that time and input the patient's information into prescription manager 7310 using user device 7340. This causes a record of the patient to be established with prescription manager 7310. For purpose of illustration, once the intake process is completed, a user account can be established for the patient.

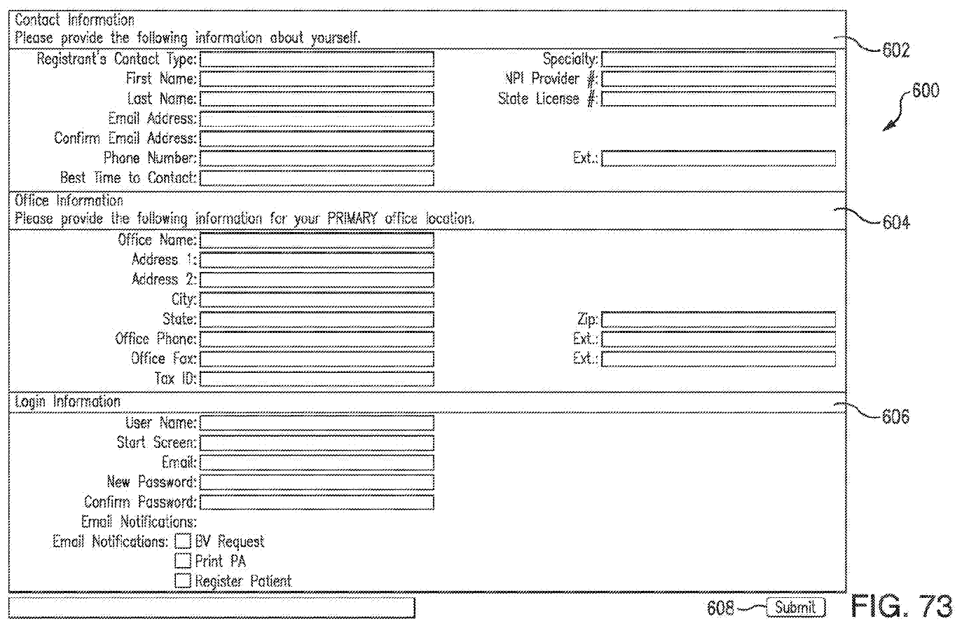

FIG. 73 is a screenshot of an exemplary HCP registration window 600 typically displayed on display device 506 of HCP system 500 (shown in FIG. 5). In other embodiments, HCP registration window 600 can be displayed on any other suitable display device, such as a display device on client systems 114, workstations 138, 140, 142, 146, or 154, mobile device 158, or the like. Registration window 600 includes a contact information window 602, an office information window 604, and a login information window 606. To register a HCP in an exemplary embodiment of the system disclosed herein, the physician contact information (e.g., name, license number, or the like) is entered in contact information window 602, office information (e.g., name, address, or the like) is entered in office information window 604, and login information (e.g., username, password, or the like) is entered in login information window 606. Once the relevant information is entered, a submit button 608 can be selected to register the HCP with the system disclosed herein. As embodied herein, registration with the system disclosed herein is performed using exemplary HCP system 500. In other embodiments, registration with the system disclosed herein is performed separate from HCP system 500, such as via a portal function.

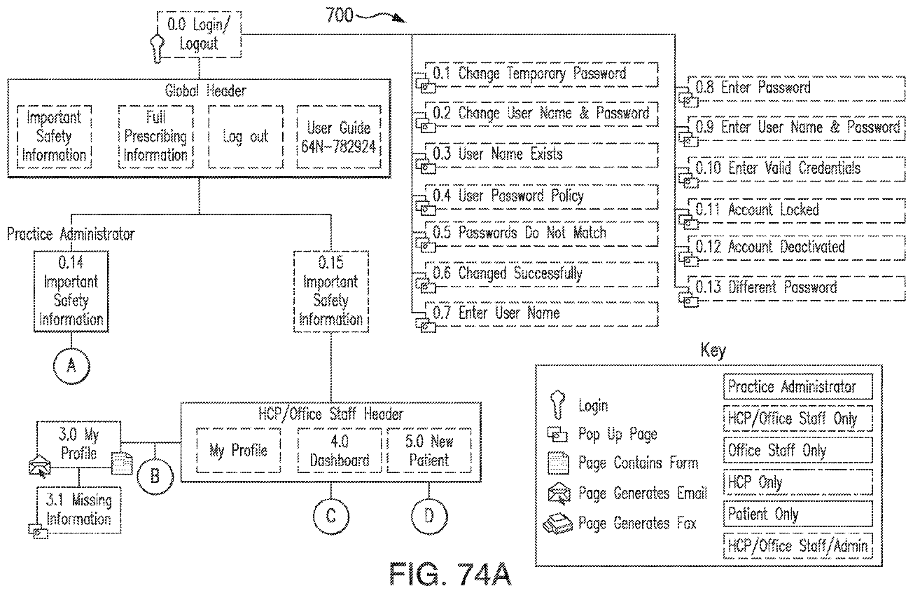

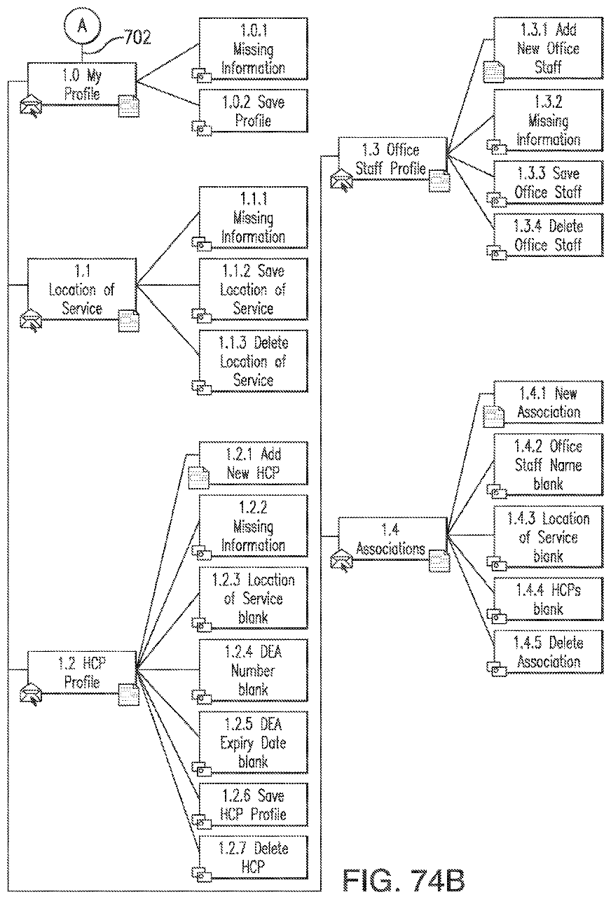

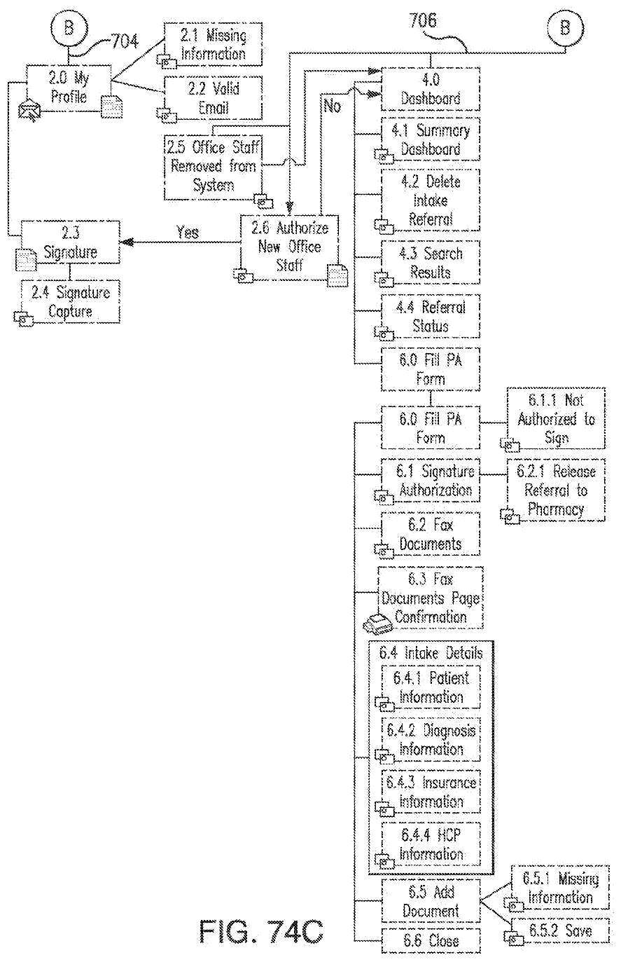

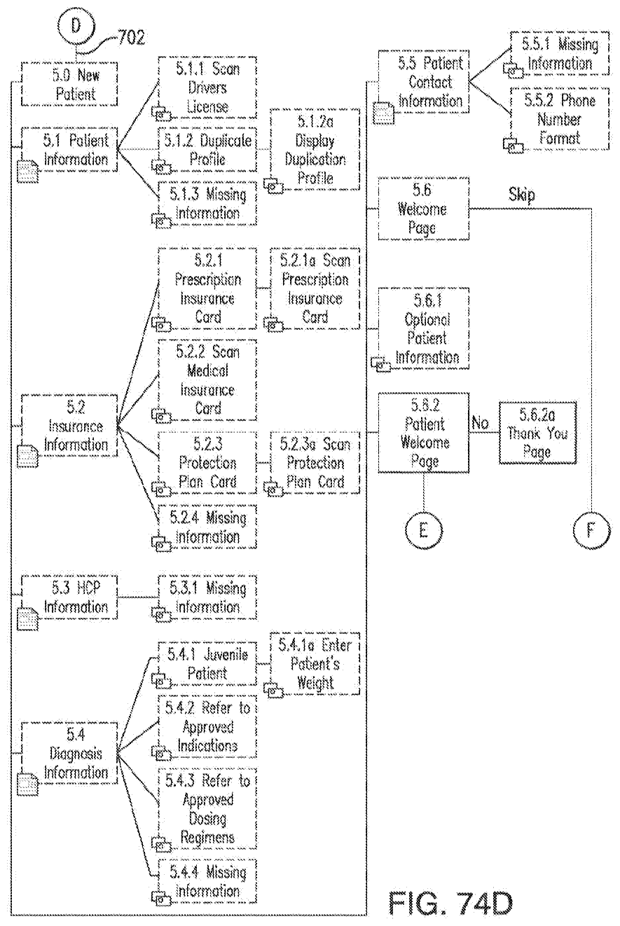

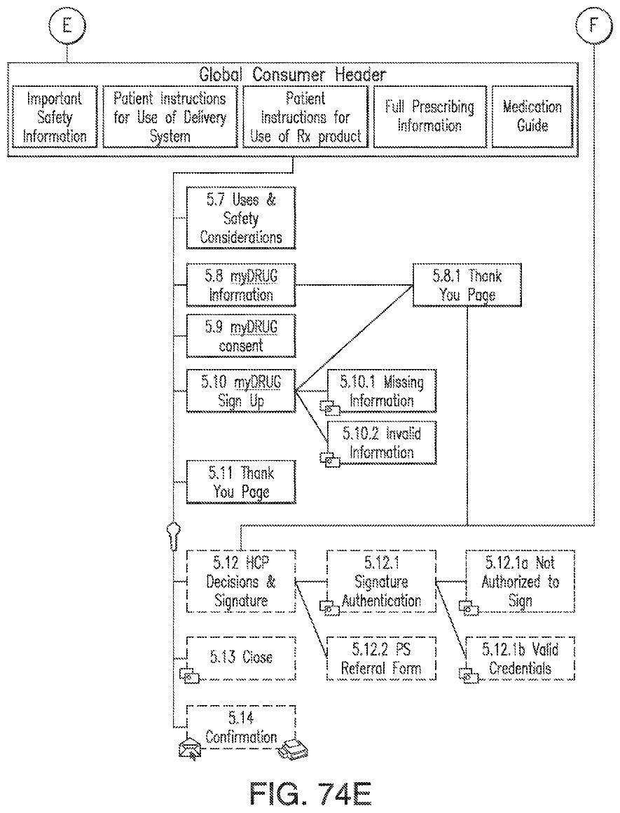

FIGS. 74A-74E illustrate a diagrammatical map 700 of an exemplary user interface in connection with the system disclosed herein implemented according to the systems and methods of the present disclosure. FIG. 74B depicts a diagram of an exemplary user interface for an administrator. On path 702 the administrator is presented with several administrative options. FIGS. 10-17 are screenshots of windows along path 702.

FIG. 10 is a screenshot of an administrator profile window 1000. Administrator profile window 1000 displays general profile information about the currently logged-in administrator. The administrator can be a practice administrator authorized to administrate the system embodied herein with respect to one or more portions (including all) of a practice, and or a system administrator who is authorized to administrate the system disclosed herein with respect to an entire practice or practices, including setting up profiles, credentials, or the like for one or more practice administrators. The profile information can be edited and saved to update/change the user's profile information. Profile window 1000 can be displayed by the administrator at any time by selecting profile tab 1002. If the user selects a tab other than profile tab 1002, a different window, described below, is displayed.

Selection of location of service tab 1004 causes display of location of service (LOS) window 1100, shown in FIG. 11. LOS window 1100 displays information about one or more facilities. Thus, for example, a medical practice that includes more than one office can have each facility name, address, telephone number, fax number, or the like stored and displayed in LOS window 1100. In an exemplary embodiment, the stored information is used to populate one or more forms by the system disclosed herein.

FIGS. 12 and 13 are screenshots of an HCP window 1200 accessible by selecting HCP profile tab 1006. HCP window 1200 displays information, about one or more HCPs. This information is presented in summary form in HCP window 1200. More detailed information can be edited for HCPs already entered into the system disclosed herein and/or entered when adding a new HCP to the system disclosed herein. FIG. 13 is a screenshot of HCP window 1200 expanded by selection to add a new HCP to show detailed information about an HCP that can be entered including, for example, name, address, LOS, work and cell phone numbers, specialties, license numbers, or the like. The same information can be edited from HCP window 1200 for an HCP already entered in an exemplary embodiment of the system disclosed herein by selecting an existing HCP and selecting to edit the HCP profile.

Profiles for HCP staff members can also be viewed, created and edited by the administrator by selecting office staff profile tab 1008. This selection accesses staff profile window 1400, shown in FIGS. 14 and 15. Summary staff profile information is displayed in staff profile window 1400. More detailed information can be edited for staff already entered into the system disclosed herein and/or entered when adding a new staff member to the system. FIG. 15 is a screenshot of staff profile window 1400 expanded by selection to add a new office staff member to show detailed information about an staff member that can be entered including, for example, name, address, email address, work phone number, and cell phone number. The same information can be edited from staff profile window 1400 for an office staff member already entered in the system by selecting an existing office staff member and selecting to edit the profile.

Associations within a practice can be viewed, added and/or edited by selecting association tab 1010. Associations within the practice include which staff members work at which location of the practice and with which HCPs. The selection of association tab 1010 accesses association window 1600, shown in FIGS. 16 and 17. Summary association information is displayed in association window 1600. More detailed information can be edited and/or newly entered. FIG. 17 is a screenshot of association window 1600 expanded by selection to add a new association. From the expanded association window 1600, the administrator can select an office staff member, select which location(s) at which the staff member works, and select the HCP(s) with whom the staff member works. The same information can be edited from association window 1600 for an office staff member for whom associations have already been entered by selecting an existing office staff member and selecting to edit the associations.

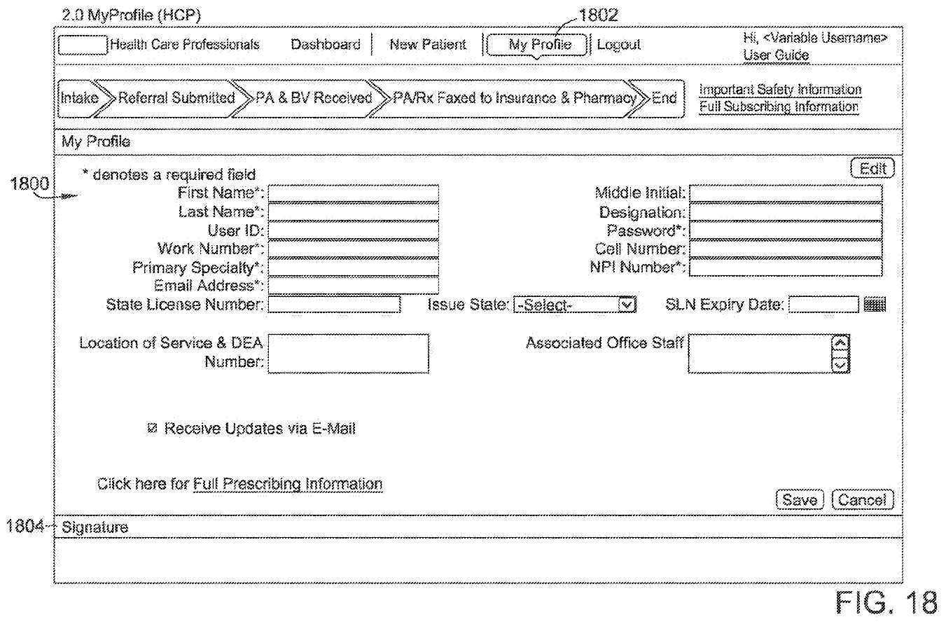

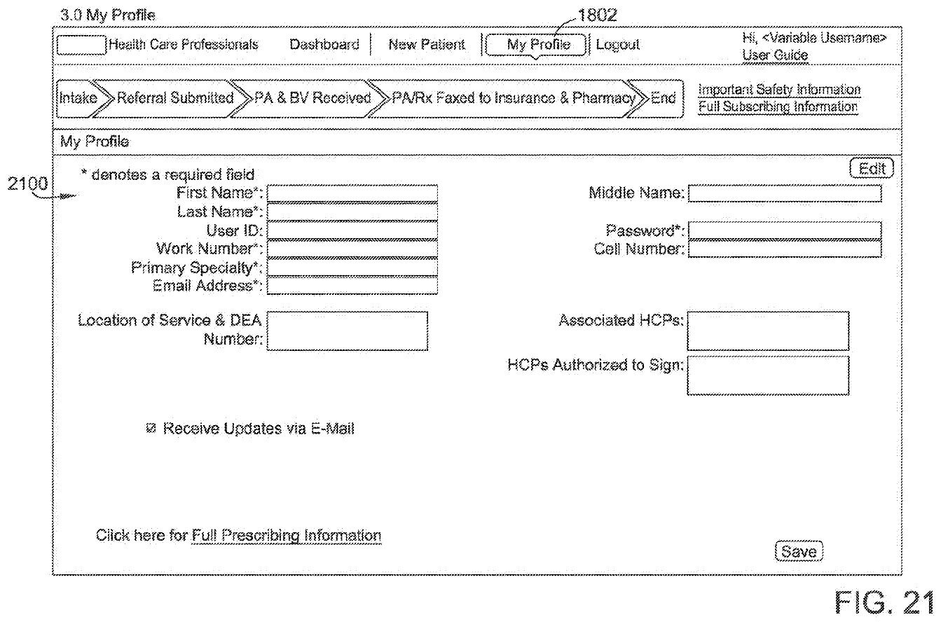

As noted above, FIG. 73 is an exemplary HCP registration window. When the user is a registered HCP or office staff member, the user can be presented different options to the user than are presented to the administrator. In general, the user is presented with the option to proceed down profile path 704 (shown in FIG. 74C) to the user's profile, proceed to dashboard path 706 (shown in FIG. 74C), or to proceed to new patient path 708 (shown in FIG. 74D). Within each path 704-708 some pages are accessible only by HCP, some pages are accessible only by office staff, and some pages are accessible by staff and the HCP. FIGS. 18-20 are screenshots of some of the windows along profile path 704 when the user is logged in as an HCP, while FIG. 21 is a screenshot of a window along path 704 when the user is logged in as an office staff member.

FIG. 18 is a screenshot of an HCP profile window 1800 displayed to an HCP user of the system disclosed herein when the user selects profile button 1802. HCP profile window 1800 displays information about the logged in HCP. The information includes, for example, name, address, LOS, DEA number, password, work and cell phone numbers, specialties, license numbers, or the like. Information may be edited by the HCP and/or entered when not already entered into the system. In an exemplary embodiment, associated office staff and LOS information may not be edited by the HCP and is only displayed to the HCP on profile window 1800. Changes to and entry of such information can be made by the administrator.

By selecting signature tab 1804, the user can access a signature window 1900 shown in FIG. 19. From signature window 1900, the user can view and/or create an electronic signature that may be attached to documents created using the system disclosed herein including, for example, pharmacy referrals, prescription documents, and PA forms. As used herein, an electronic signature is an electronic representation of a handwritten signature. The currently stored electronic signature, if applicable, is displayed in signature window 1900. If the user desires to create a new signature, either for the first time or to replace the currently saved signature, the user selects to capture the signature and pop-up window 2000, shown in FIG. 20, appears over signature window 1900. The user can then physically sign his/her signature for capture by the system, such as on display 506 utilizing a touch screen stylus. In other embodiments, the user can physically sign on a separate signature capture device, and/or can sign using a device other than a touch screen stylus. The captured signature is displayed in pop-up window 2000. The captured signature displayed in pop-up window 2000 may be accepted and saved, or the user can clear the signature and capture his/her signature again. In another embodiment, the user can capture a signature using a digital imaging device such as a digital camera and upload the captured signature image to the system.

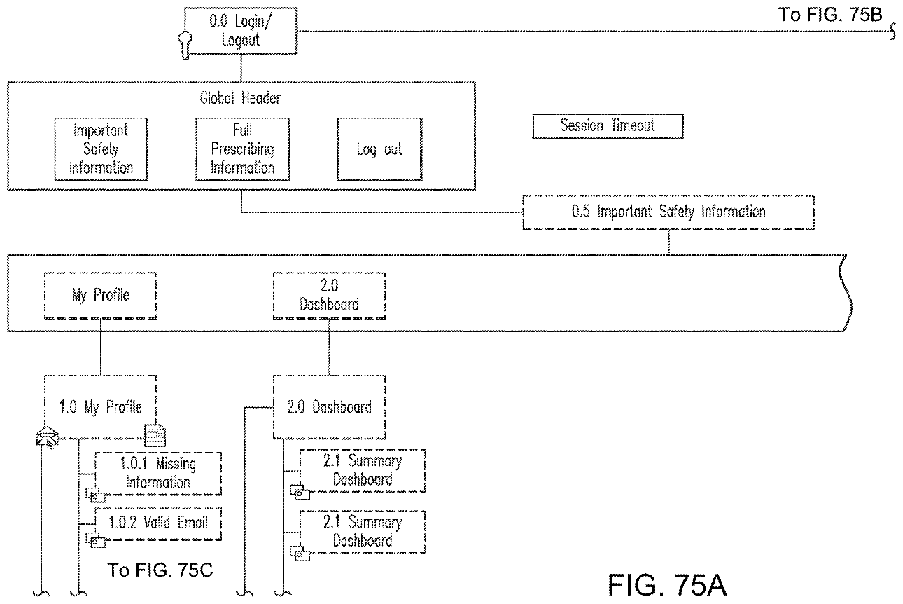

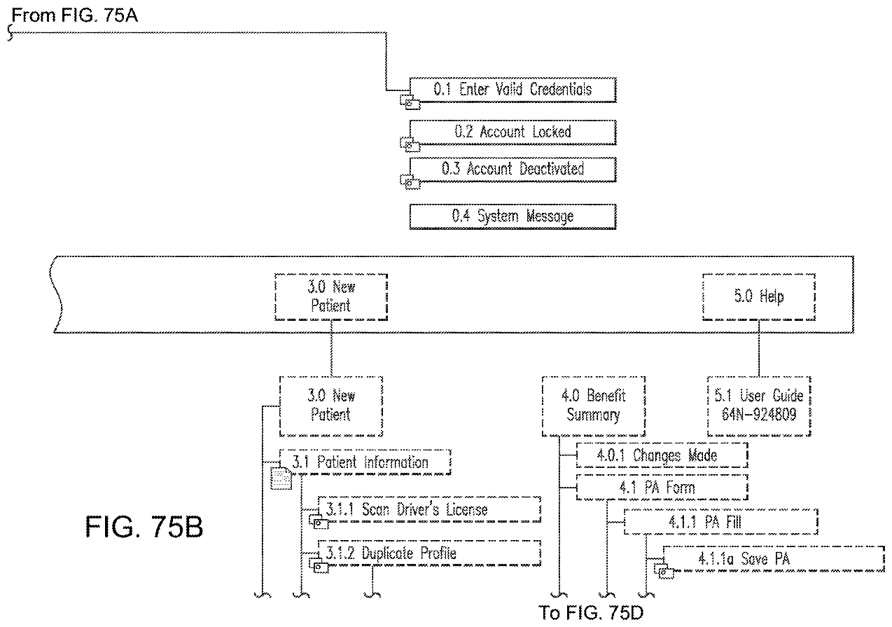

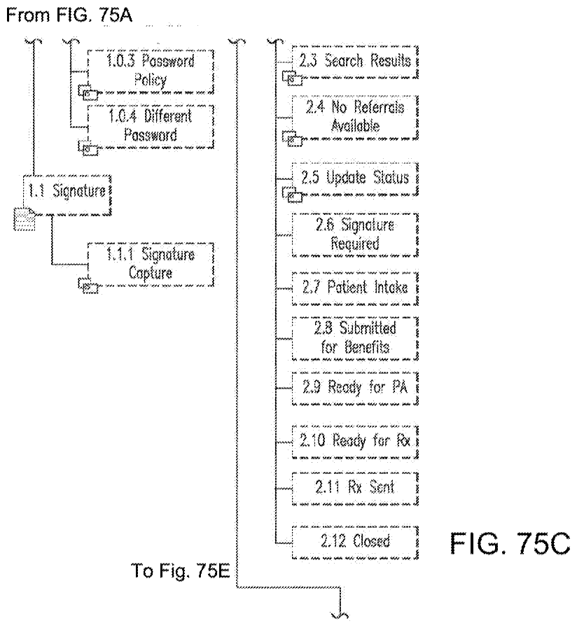

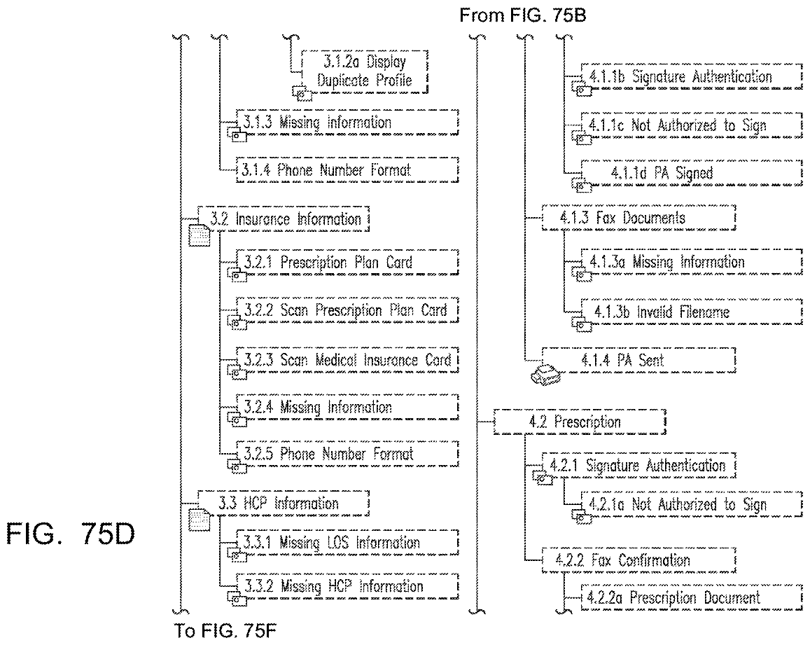

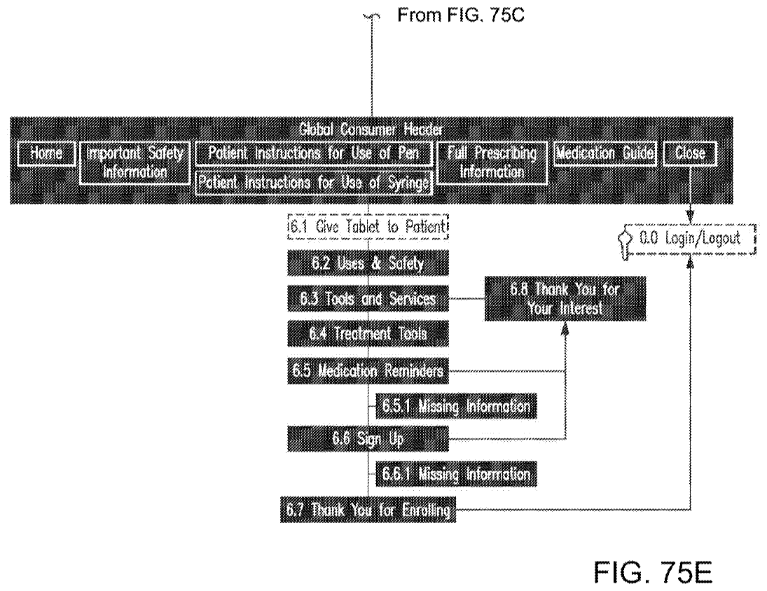

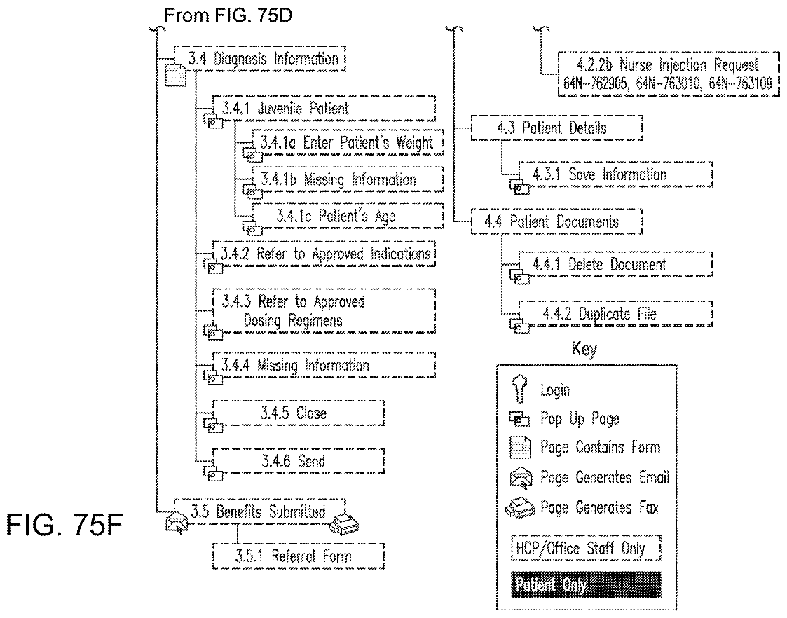

FIG. 21 is a screenshot of a staff profile window 2100 displayed to a staff user of the system when the user selects profile button 1802. Staff profile window 2100 displays information about the logged in staff member. The information includes, for example, name, user ID, password, email address, work and cell phone numbers, LOS, associated HCPs, HCPs for whom the staff member is authorized to sign documents, or the like. Information can be edited by the staff member and/or entered when not already entered into the system. In the exemplary embodiment, associated HCPs, LOS information, and HCPs for whom he staff member is authorized to sign may not be edited by the staff member and is only displayed to the staff member on profile window 2100. Changes to and entry of such information are made by the administrator. For purposes of illustration and not limitation, FIGS. 75A-75F illustrate another exemplary diagrammatical map of an exemplary user interface in connection with the system disclosed herein implemented according to the systems and methods of the present disclosure.

As noted above, prescription manager (including, for example and without limitation, various embodiments of the prescription manager, depicted in the figures as 10, 7310, and 112) can also manage the acquisition of certain patient information (“patient intake”) (21), either alone or in combination with one or more user devices (including, for example and without limitation, various embodiments of the user devices, depicted in the figures as 60, 500, 522, and 114).

As disclosed herein, for illustration, to establish a record or account for the patient (11), various types of information concerning the patient can be required, which can be referred to as “patient intake” (21). Patient information can include, for example and without limitation, name, address, gender, birth date, social security number, insurance provider, insurance number, preferred pharmacy, preferred healthcare facility (e.g., hospital or clinic), name of the primary care physician, and so on. There are various ways to obtain the necessary patient information. As an example, suppose that a patient wishes to establish an account with prescription manager 7310 when visiting a healthcare provider (e.g., having the healthcare provider perform the intake process for the patient using user device 7340). If user device 7340 includes a card scanner, the patient's driver's license, identification card, and/or insurance card(s) can be scanned (e.g., front and/or back of the card or both sides) and the patient's information can be extracted from the scanned images automatically (e.g., using OCR). As another example, if user device 7340 includes a camera, digital photos of the patient's driver's license, identification card, of the patient (e.g., the patient's face), and/or insurance card(s) can be taken, and the patient's information can be extracted from the digital photos automatically (e.g., using image recognition). As used herein, the term “scanning device” can refer to, for example, an optical scanner, such as a card scanner, as well as a camera suitable to acquire digital photos. One of ordinary skill in the art will appreciate that such a scanning device need not be directly coupled to a particular user device (e.g., user device 7340). For example, the scanning device can be coupled to any suitable computing device or processor, which can be coupled to the user device 7340 so as to transmit the scanned images. As a third example, the patient's information can be manually typed into user device 7340 (e.g., using a virtual or physical keyboard).

As disclosed herein, for illustration, the user interface provided by prescription manager 7310 can include a series of screens (e.g., web pages), accessible with a user device (e.g., user device 7340 or 7350), that guides the healthcare provider through the patient intake process (21) or guides the patient through the account registration process. At various screens, the healthcare provider or the patient can input various types of information to be saved with prescription manager 7310 in the patient's account or transmitted to an EMR to be saved in the patient's record(s) there (e.g., in data store 7360).

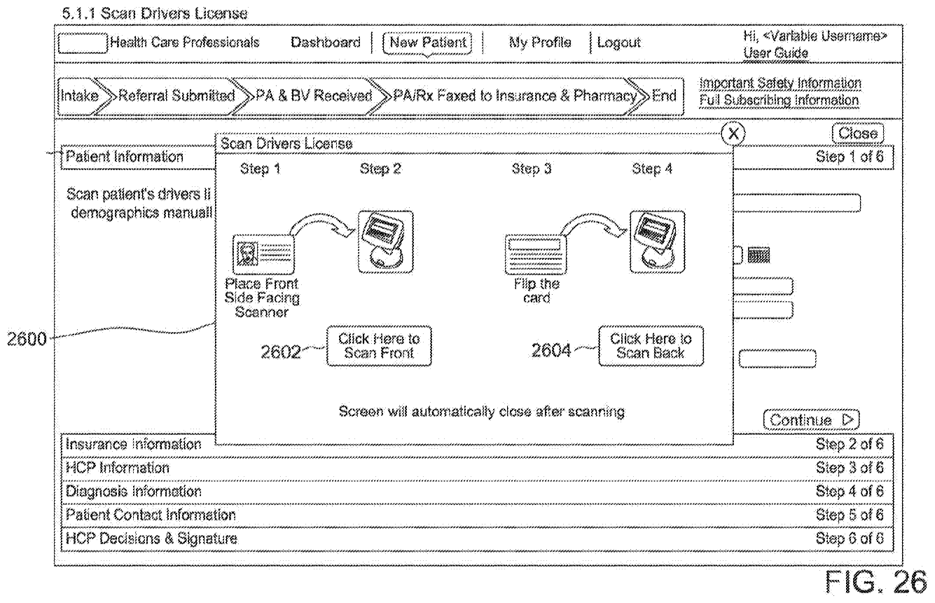

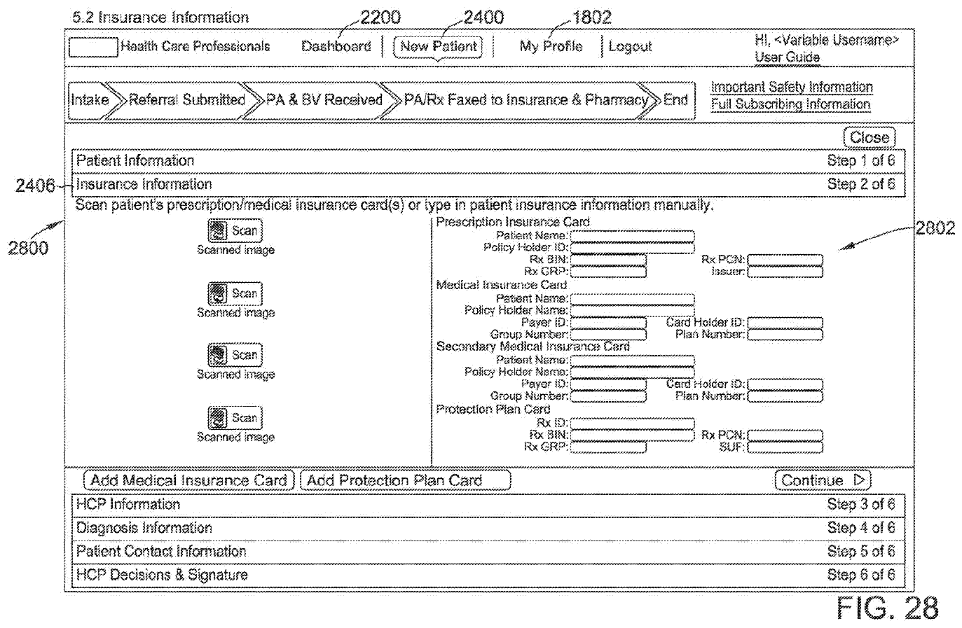

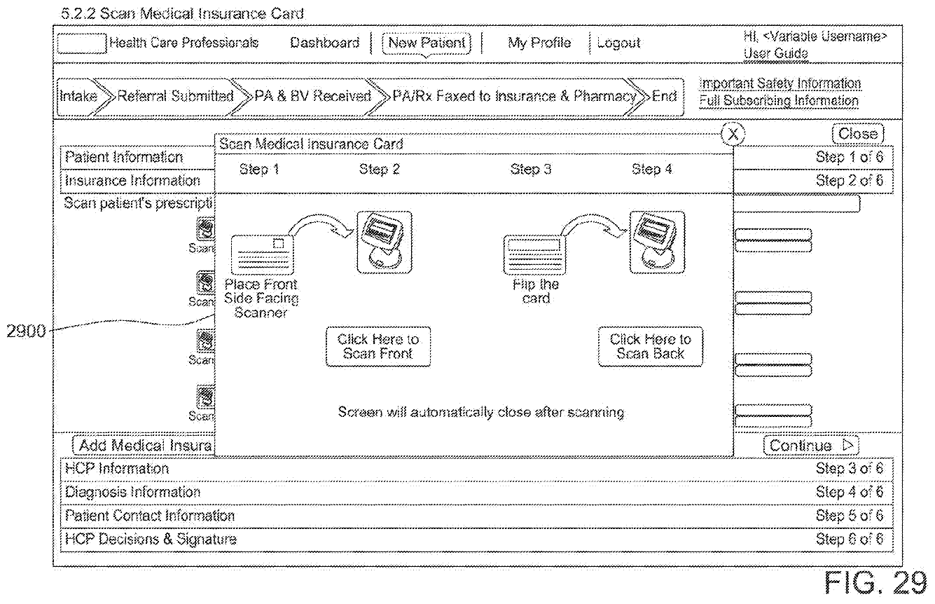

FIGS. 25-30 illustrate example screens that guide a healthcare provider to scan a patient's identification documents in order to extract the necessary patient information automatically when performing the intake process for a patient (e.g., using user device 7340 that includes a card scanner). For example, the healthcare provider can activate “Scan” icon 2504 illustrated in FIG. 25 to begin the card scanning process. In FIG. 26, icons 2602 and 2604 can guide the healthcare provider to scan both the front and back of the patient's driver's license. In FIGS. 28 and 29, the healthcare provider can be guided to scan the front and/or back or both sides of the patient's medical insurance card or prescription card. The information extracted from these cards can be used to automatically populate (i.e., fill in) the various fields 2502 illustrated in FIG. 25 and fields 2802 illustrated in FIG. 28, which relate to the patient's information. The patient can review the individual field values to make sure that the information is correctly extracted from the scanned images of the cards, and manually correct any field values when needed.

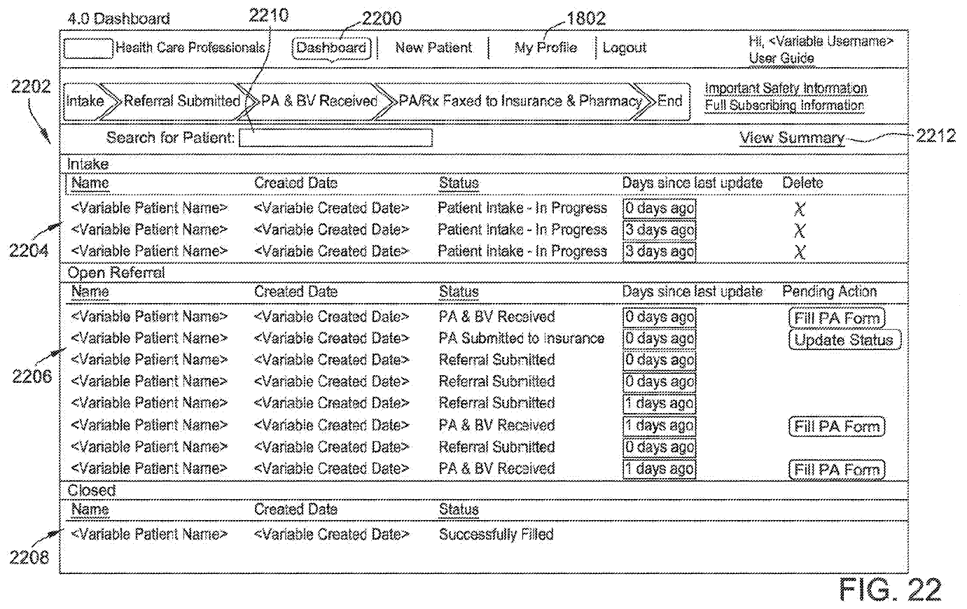

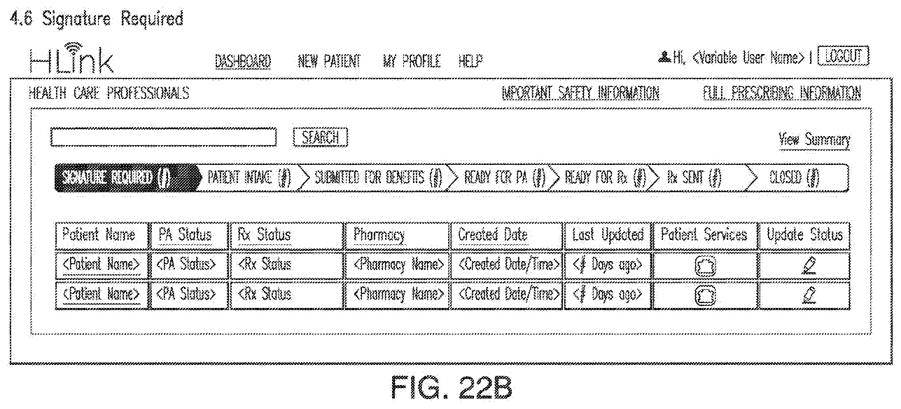

Once the patient's information is entered into user device 7340, user device 7340 can encrypt and send the patient's information to prescription manager 7310. Prescription manager 7310 can in tum create an account for the patient and store the patient's information in the account (e.g., in data stores 7312). The information can be stored in an encrypted format, and can be temporarily decrypted for display or processing purposes, as disclosed herein. The patient can use the user name and password associated with the account to log into his/her account in the future. In addition, the healthcare provider can access the patient's information through his/her own account (e.g., from screen 2202 illustrated in FIG. 22).

For purpose of illustration and not limitation, reference is now made to a situation where a patient is visiting a healthcare provider (e.g., doctor, nurse, or other types of medical profession), and the healthcare provider decides to prescribe the patient a prescription medication (i.e., a prescription product), such as HUMIRA® developed and manufactured by Abbott Biotechnology Ltd, or refer the patient to another healthcare provider (i.e., a prescription service), such as a specialist, which is supported by the system and method disclosed herein. The healthcare provider can utilize system and method to obtain authorization from the patient's insurance provider for the prescription product or service, when needed.

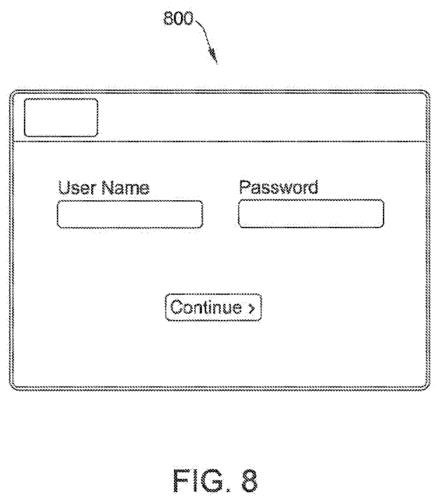

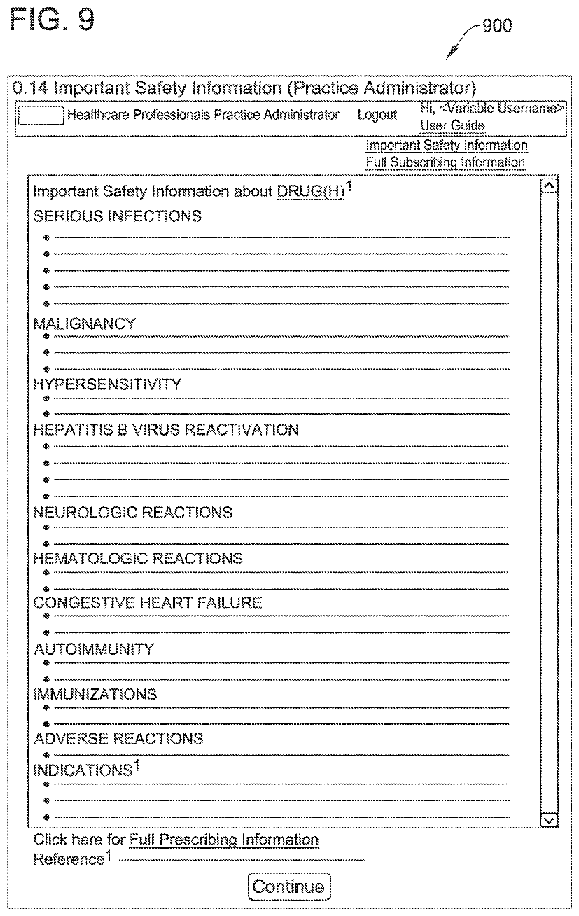

As described above, As disclosed herein, for illustration, in order to utilize system 7300, both the healthcare provider and the patient would need to establish their respective user accounts with prescription manager 7310. Upon deciding to prescribe a specific product or service to the patient, the healthcare provider can log into his/her account with prescription manager 7310. To do so, the healthcare provider can, for example, access a login screen (e.g., a login web page at the website corresponding to prescription manager 7310) using user device 7340, and provide his/her user name and password associated with the account. An example login screen 800 is illustrated in FIG. 8, which can be part of the web-based user interface provided by prescription manager 7310. Once logged into his/her account, the healthcare provider can access functions implemented and supported by prescription manager 7310 to perform various patient-care activities. As for the patient, again, if the patient does not already have an account with prescription manager 7310 at the time visiting the healthcare provider, the healthcare provider can log into his/her own account and then perform the intake process for the patient as needed to establish a user account for the patient. On the other hand, if the patient has already been inputted into system 7300 and has a user account with prescription manager 7310, it is not necessary to perform an intake process for the patient. Instead, As disclosed herein, for illustration, the healthcare provider can log into his/her own account and retrieve the patient's information through his/her account (e.g., from screen 2202 in FIG. 22) and verify the information stored with prescription manager 7310 with the patient.

As embodied herein, screens (e.g., web pages) can be provided by prescription manager 7310, as part of its web-based user interface, to allow the healthcare provider to browse or search for patients in system 7300. FIG. 22 illustrates an example patient-information screen 2202. In this case, patients whose intake processes are in progress can be listed in area 2204. Patients who have open referrals can be listed in area 2206. In addition, the healthcare provider can search for a specific patient by inputting the patient's name in text field 2210. Once the patient's record in system 7300 has been located, the healthcare provider can continue with the prescription process. For purposes of illustration and not limitation, FIG. 22B illustrates another exemplary patient information screen including a signature requirement in accordance with an embodiment of the disclosed subject matter.

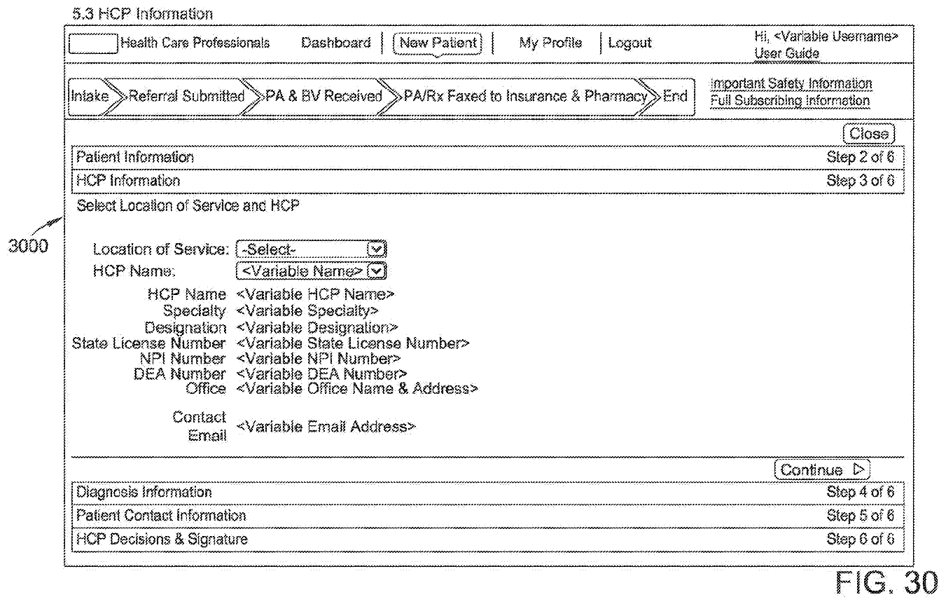

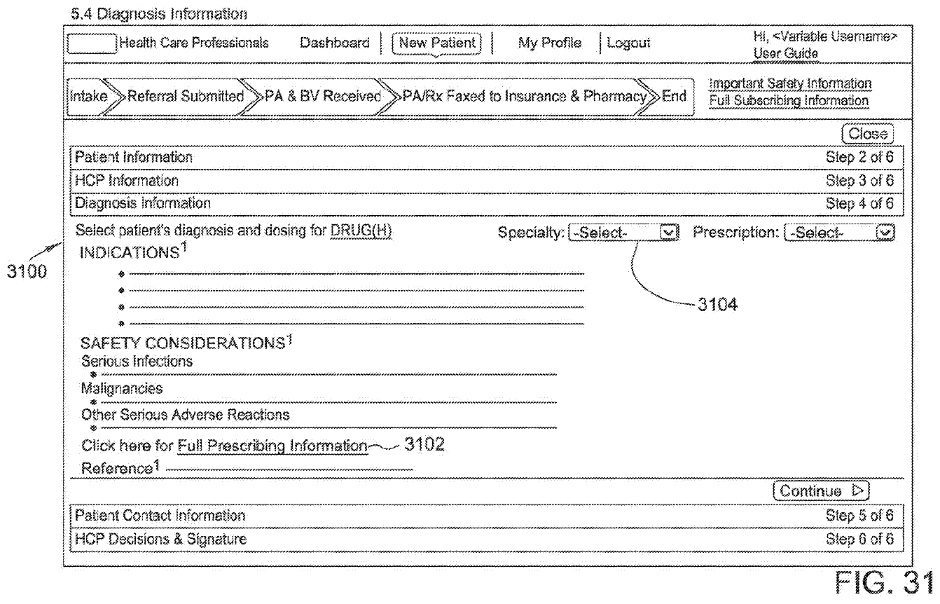

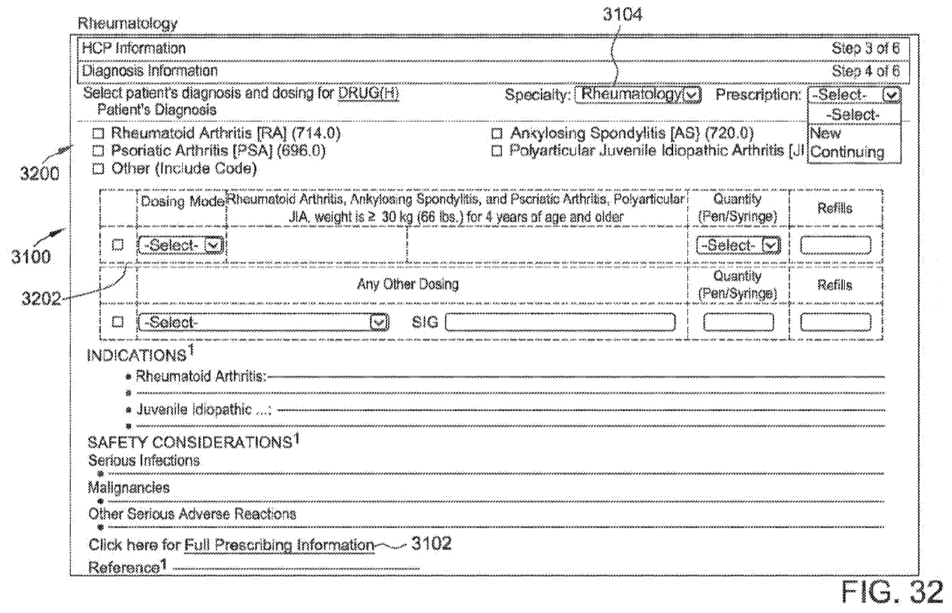

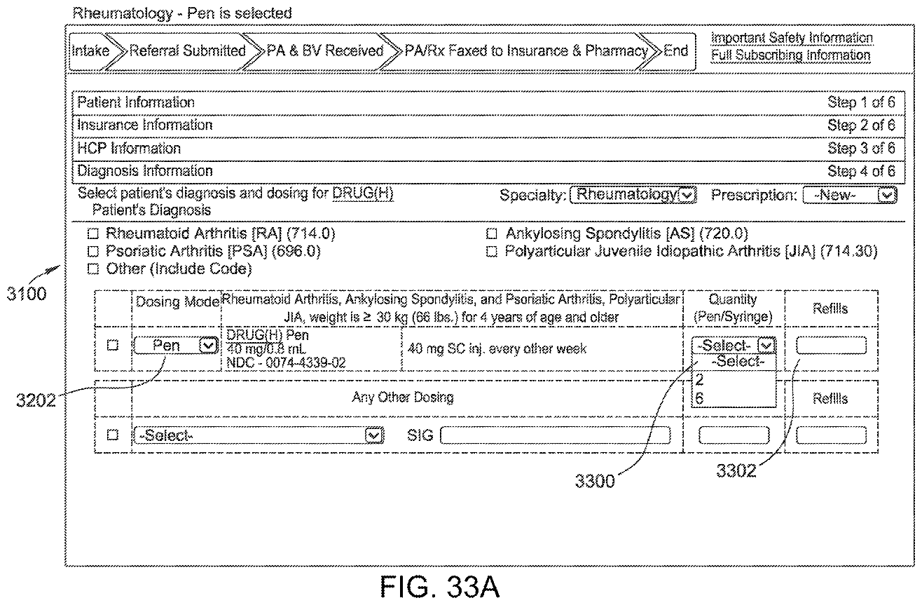

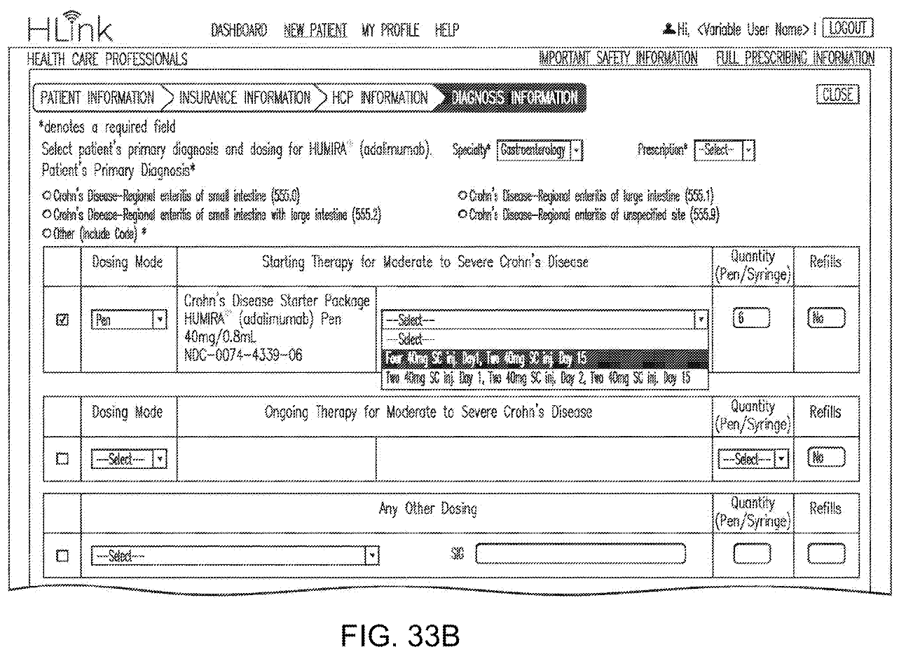

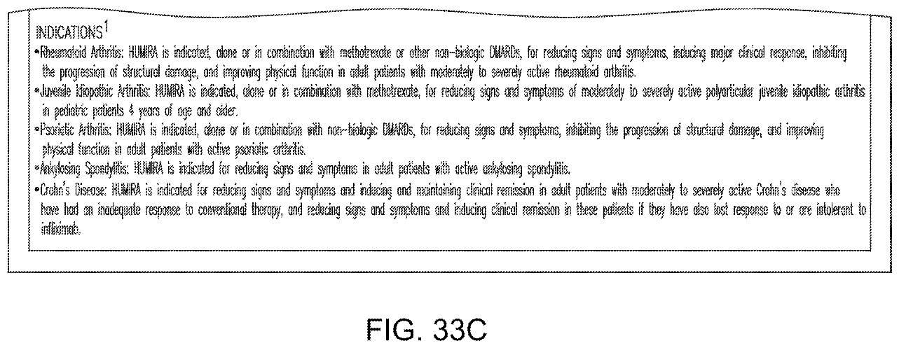

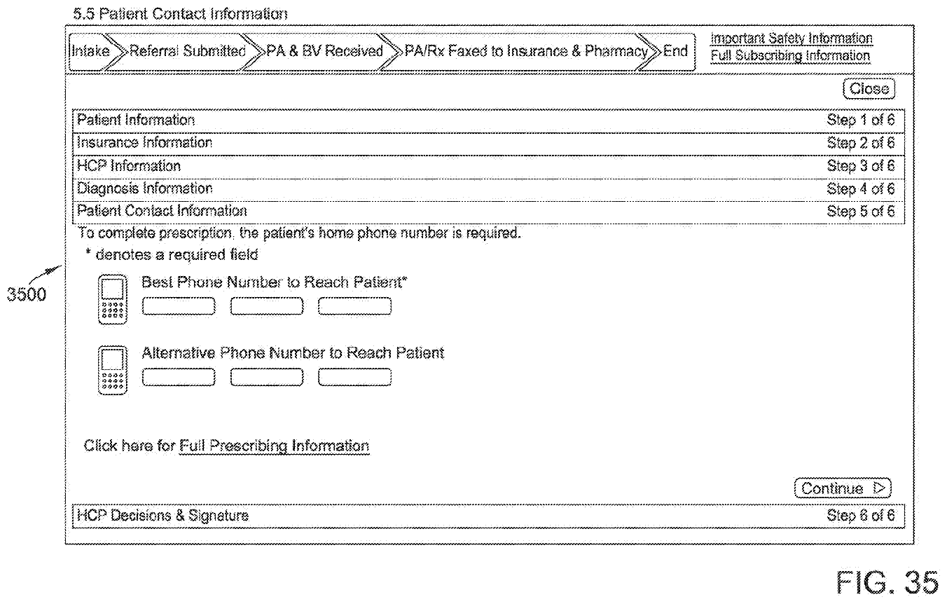

As disclosed herein, for illustration, the healthcare provider can input information concerning the prescribed product or service using user device 7340. For purpose of illustration, screens can be provided by prescription manager 7310, as part of its web-based user interface, to guide the healthcare provider to input prescription information. FIGS. 30-35 illustrate example screens that guide the healthcare provider to enter prescription information in user device 7340. For example, the healthcare provider select the “Location of Service” and “HCP Name” (i.e., the name of the healthcare provider) for the patient from screen 3000 illustrated in FIG. 30. This can be the healthcare provider's own office location and name. From screen 3100 illustrated in FIG. 31, the healthcare provider can type in or select from a pre-established list (e.g., a pull-down menu) the patient's diagnosis and the specific product or service to be prescribed to the patient. The system and method can be configured to support only one prescribed product, or allow for the selection from a number of different prescribed products. If the healthcare provider prescribes a medication to the patient, there can be a recommended dosage displayed on screen 3100 illustrated in FIGS. 32 and 33A-33C. The healthcare provider can select the recommended dosage or override it and enter a different dosage. Similarly, there can be a recommended frequency for administering the medication displayed on screen 3100 illustrated in FIGS. 33A-33C, which the healthcare provider can override if he/she so chooses. There can be safety considerations displayed on screen 3100 illustrated in FIG. 31. The healthcare provider can select or specify the form of the medication (e.g., pills, injections, or the like). The healthcare provider can specify whether this is a medication newly prescribed to the patient, a continuing prescription, or the patient is restarting on the medication after a break. The patient can, through the healthcare provider, select a preferred pharmacy where the patient can purchase and pick up the medication. If the healthcare provider refers the patient to a specialist, the healthcare provider can specify the name and location of the specialist, the practice field of the specialist, or the treatment needed for the patient. In addition, the patient can provide one or more telephone numbers (e.g., as illustrated in FIG. 35) or other contact information (e.g., an email address) so that the pharmacy or the specialist may contact the patient (e.g., when the medication is ready for pickup or set up an appointment with the specialist).

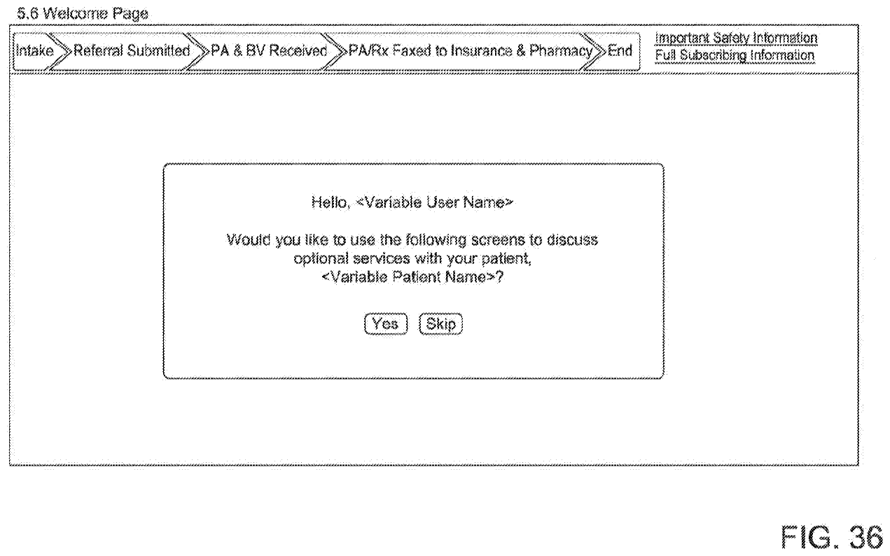

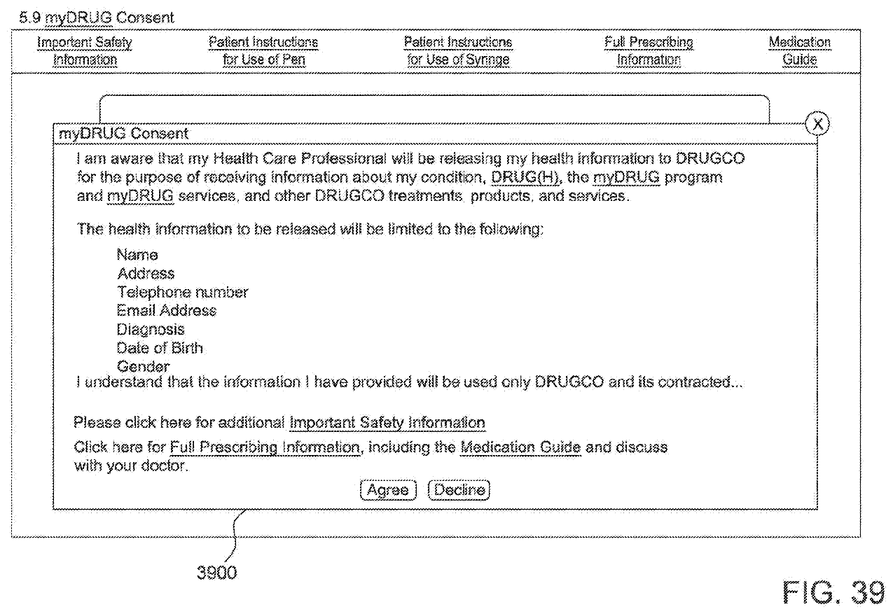

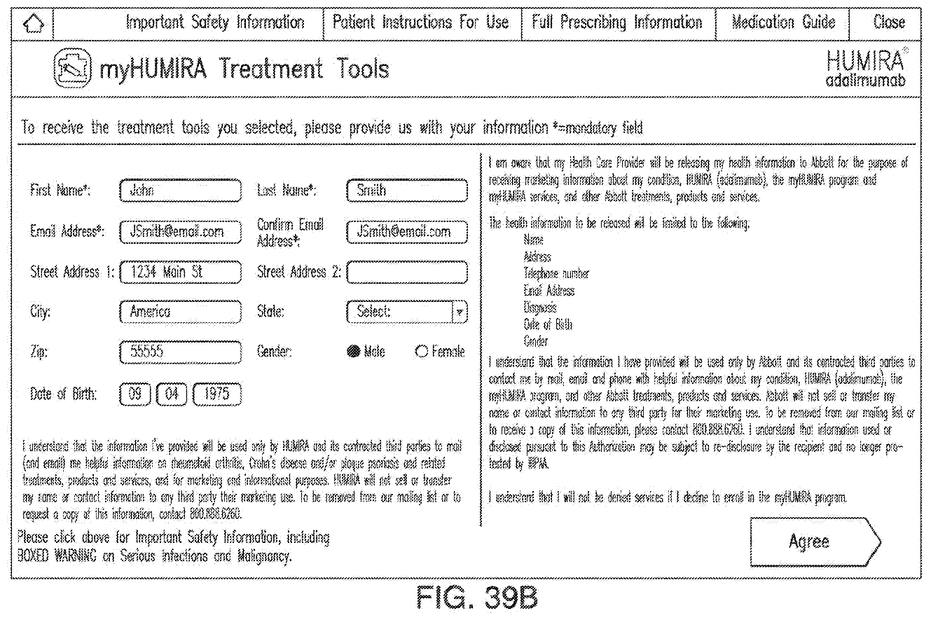

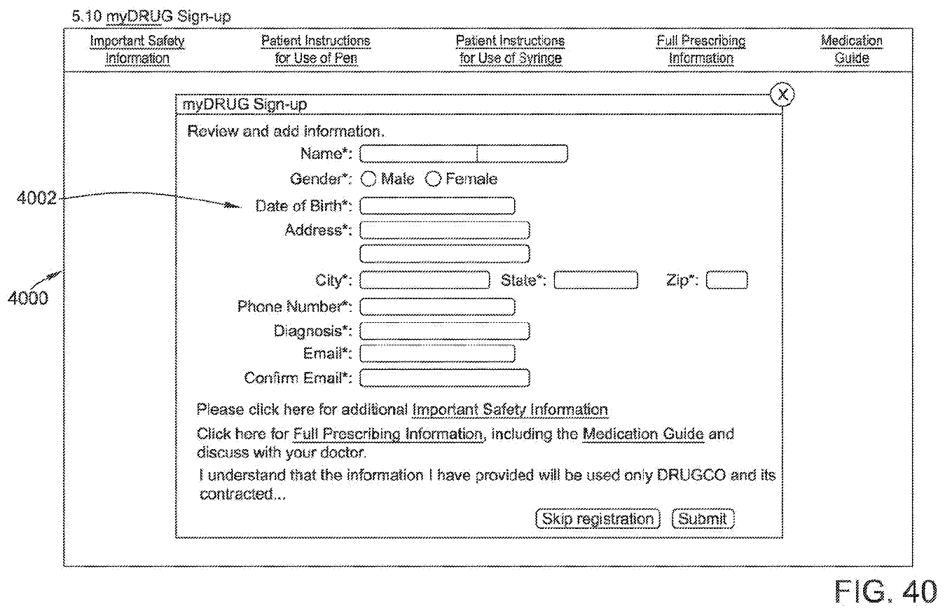

For purpose of illustration, the healthcare provider can discuss optional services, which can be relevant to the patient's treatment, with the patient. Again, screens can be provided by prescription manager 7310 to guide the healthcare provider through such a discussion. FIGS. 36-40 illustrate example screens that guide the healthcare provider to discuss optional services with the patient. For example, the screens can display those optional services specifically available or relevant to the patient (e.g., product support services provided by the manufacturer of the medication prescribed to the patient), as illustrated in FIG. 36. The patient can select and sign up for specific services, with help from the healthcare provider using user device 7340, as illustrated in FIG. 40, and give the necessary content required by these services, as illustrated in FIG. 39. In an exemplary embodiment, optional services can be discussed, and or displayed via guiding screens, at any number of instances. For example, optional services can be discussed after entry of prescription information, before or after generating and/or transmission of a prescription document or medical order document, before or after benefits verification and/or prior authorization, as discussed in more detail below.

In some cases, the healthcare provider can allow the patient to enter information directly into user device 7340. For example, from screen 3500 illustrated in FIG. 35, the patient can enter his/her contact information (e.g., telephone numbers). The patient can select a specific pharmacy from where the patient prefers to obtain the prescription medication. If desired, when the healthcare provider hands user device 7340 to the patient, the patient can be locked out of certain screens as a security measure. For example, the patient can not be able to access those screens where the healthcare provider specifies prescription medications and their dosages for the patient. This prevents the patient from changing (e.g., increasing) the dosages of the prescription medications or changing the medications or adding other medications for himself/herself. For purpose of illustration, there can be a button on user device 7340 or an icon included in one of the screens that once activated, causes certain screens to be locked from further access. Before handing user device 7340 to the patient, the healthcare provider can activate the button or the icon. Once, the patient returns user device 7340 back to the healthcare provider, the healthcare provider can unlock these screens (e.g., by inputting user name and password to user device 7340).

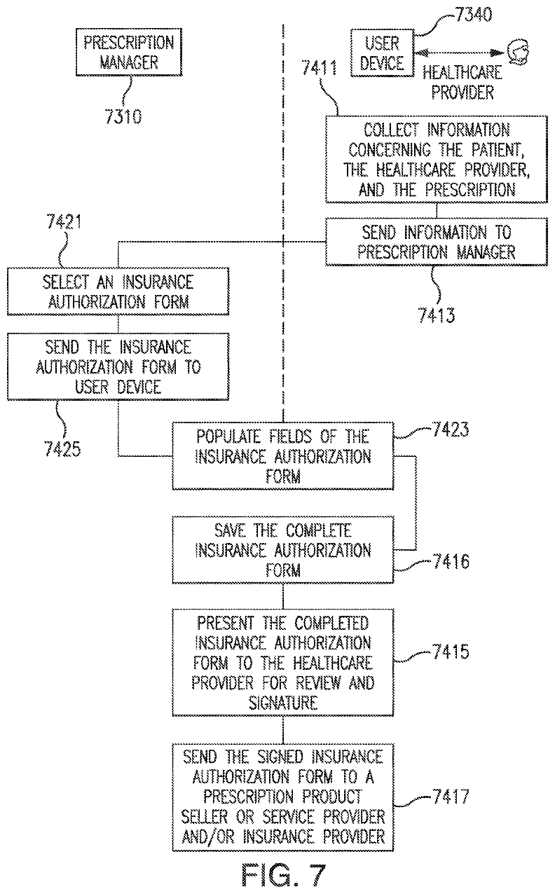

With reference to FIG. 7, As disclosed herein, for illustration, at 7411, user device 7340 can collect all the information entered into it by the healthcare provider and optionally by the patient, which can include information concerning the patient (e.g., the patient's name, insurance number, or user name), the healthcare provider (e.g., the healthcare provider's name or user name), and the prescription product or service. At 7413, user device 7340 can optionally encrypt the information and send the information to prescription manager 7310 (e.g., through a HTTPS connection). For example, the healthcare provider can click a “SUBMIT” button displayed on one of the screens to cause user device 7340 to begin sending the information to prescription manager 7310.

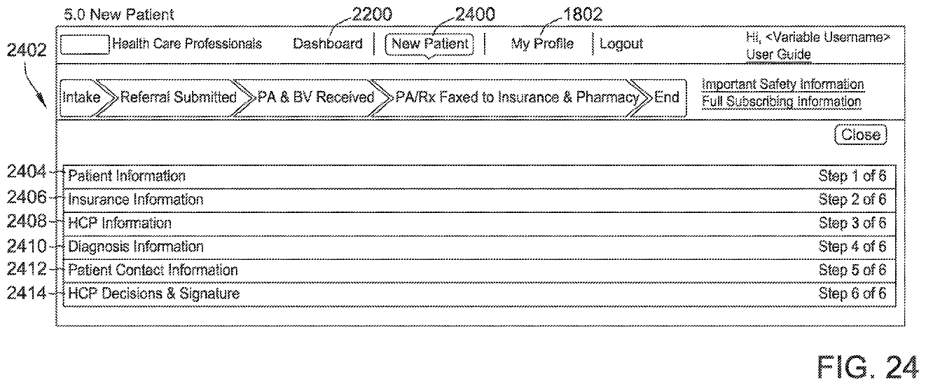

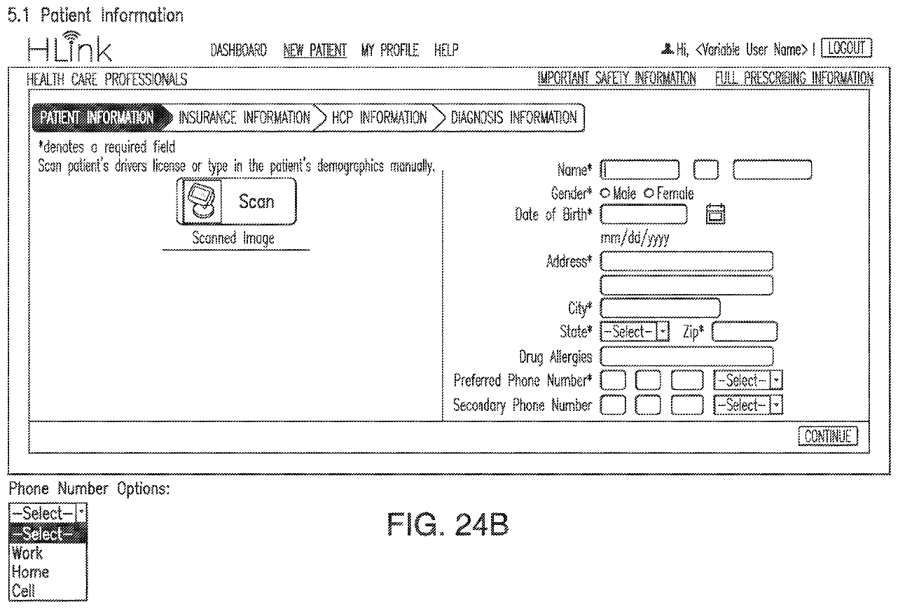

For purposes of illustration and not limitation, new patient intake using an exemplary embodiment of the system disclosed herein will be described with reference to FIGS. 24-43. This process can be performed by the HCP, a staff member, or a combination of the HCP and one or more staff members. Accordingly for FIGS. 24-43, unless otherwise specified, a user can be an HCP or a staff member.

Referring initially to FIG. 24, when the user selects new patient button 2400, new patient page 2402 is displayed. New patient page 2402 includes a patient information tab 2404, an insurance information tab 2406, an HCP information tab 2408, a diagnosis information tab 2410, a patient contact information tab 2412, and an HCP decision and signature tab 2414. These six tabs access windows applicable to six steps in the new patient intake process. In the exemplary embodiment, computing device 502 (shown in FIG. 5) enters a first input mode. In the first input mode, computing device 502 receives data entered by HCP. For purposes of illustration and not limitation, FIG. 24B illustrates another exemplary patient information screen in accordance with an embodiment of the disclosed subject matter.

Selecting patient information tab 2404 opens patient information window 2500 shown in FIG. 25. Patient information window 2500 includes fields 2502 for patient information (e.g., name, address, or the like). Information can be manually input into patient information window 2500 using, for example, keyboard 508 and/or touch screen display 506. Alternatively, or additionally, the user can select to scan a patient identification document, e.g., a driver's license, to acquire the information and populate fields 2502 with the information. When the user selects scan button 2504, scanning pop-up 2600, shown in FIG. 26, is displayed over patient information window 2600. Scanning pop-up 2600 instructs the user how to scan the identification document using, for example, scanning device 504. The user scans the front and back of the identification document by placing the document on scanning device 504 and selecting a scan front button 2602 and a scan back button 2604. In the exemplary embodiment, scanning device 504 delivers the scanned image(s) of the identification document to computing device 502. Computing device 502 performs optical character recognition on the scanned images to the needed information for fields 2406 from the images of the identification document. In other embodiments, a different component of the system, such as scanning device 504, performs the optical character recognition. Additionally or alternatively, the information can be extracted by other than optical character recognition. For example, in an exemplary embodiment, a bar code or other visual data encoding element is read by the system. In still other embodiments, a non-visual data storage element, such as a magnetic stripe, an RFID chip, or the like, in the identification document is read to extract the patient information. The extracted information is used in connection with an exemplary embodiment of the system disclosed herein to automatically populate fields 2502. In the exemplary embodiment, the system stores the captured images of the identification document and displays one or more of the images in patient information window 2500. In yet another embodiment, the information can be imported into the system from an electronic medical records system.

If the user attempts to enter patient information, whether manually or via an ID scan, for which a patient profile already exists in the system, a duplicate profile pop-up 2700 is displayed. Identification information for the preexisting patient profile is displayed in pop-up 2700. The user can select to use the identified patient profile or ignore the existing profile and continue to create a new patient profile.

When the user selects insurance information tab 2406 or selects to continue from patient information window 2500, insurance window 2800 is displayed, as shown in FIG. 28. Insurance window 2800 includes fields 2802 for the patient's insurance information, also referred to herein as insurance data. More specifically, insurance window 2800 includes fields for prescription insurance information, medical insurance information, and prescription protection plan information. Not all patients will have all types of insurance and some can have more than one of a particular type of insurance. Information can be manually input into insurance window 2800 using, for example, keyboard 508 and/or touch screen display 506. Alternatively, or additionally, the user can select to scan insurance identification document(s) to acquire the information and populate fields 2802 with the information. As with scanning of a patient identification document, when the user selects to scan an insurance identification document, a scanning pop-up 2900, shown in FIG. 29, is displayed over insurance information window 2800. Scanning pop-up 2900 instructs the user how to scan the particular document using, for example, scanning device 504. The user scans the front and back of the identification document as instructed by scanning pop-up 2900. In the exemplary embodiment, scanning device 504 delivers the scanned image(s) of the identification document to computing device 502. Computing device 502 performs optical character recognition on the scanned images to the needed information for fields 2802 from the images. In other embodiments, a different component of the system, such as scanning device 504, performs the optical character recognition. Moreover, if desired, the information can be extracted by other than optical character recognition. For example, in an exemplary embodiment, a bar code, QR code, or other visual data encoding element is read by the system. In still other embodiments, a non-visual data storage element, such as a magnetic stripe, an RFID chip, or the like, in the identification document is read to extract the patient information. The extracted information can be used by the system to automatically populate fields 2802. In the exemplary embodiment, the system stores the captured images of the identification document, and displays one or more of the images in insurance window 2800.

Selecting to continue causes an HCP information window 3000 to be displayed, as shown in FIG. 30. The user can select the location of service and HCP for the patient from pull down menus. Detailed information for the selected HCP is displayed in HCP information window 3000 after a selection is made.

FIG. 31 is a screenshot of a diagnosis window 3100 displayed after a user selects to continue from HCP information window 3000. Information, e.g., indications, safety considerations, or the like, concerning the drug to be prescribed is displayed in diagnosis window 3100. Also displayed is a link 3102 to full prescribing information for the drug. In the exemplary embodiment, the prescribing HCP's specialty is preselected in a pull down menu 3104 based on the HCP's profile. In other embodiments, a specialty is not preselected and the user must select a specialty from pull down menu 3104. In the exemplary embodiment, the specialties available in pull down menu 3104 are limited to specialties for which the drug to be prescribed is prescribed. In other embodiments, additional specialties may be available and/or the particular HCP's specialty may be the only specialty available for selection. In FIGS. 32-34 rheumatology has been selected as the specialty for explanatory purposes only and is not intended to limit the exemplary embodiment to rheumatology. After the HCP's specialty is selected, diagnosis window 3100 expands as shown in FIG. 32. The patient's diagnosis is selected from a list of diagnoses 3200 for which the drug may be prescribed. As shown in FIGS. 33A-33C, the user selects the dosing mode from pull down menu 3202, and the system disclosed herein can populate the details of the pharmaceutical product for the selected diagnosis and dosing mode. In the example embodiment, the available dosing modes, also referred to as delivery devices, include syringes and injection pens. In other embodiments, any other appropriate dosing mode can be selectable and/or insertable. Appropriate dosing modes can include, for example, infusion pumps, injection pens, syringes, pills, capsules, suppositories, ingestible liquids, topical applications (including creams, lotions, patches, or the like), pumps, wearable pumps, or the like. In the exemplary embodiment, the user enters the usage frequency for the prescribed product. In other embodiments, the usage frequency can be selected by the system based on patient data, the selected dosing, and/or the selected diagnosis. The user also selects a quantity to be prescribed from pull down menu 3300 and inserts the number of refills to be prescribed in box 3302. For purposes of illustration and not limitation, FIGS. 33B and 33C illustrate another exemplary diagnosis information screen in accordance with an embodiment of the disclosed subject matter.

Certain embodiments of the system inform the user of important information, requests additional information, and/or limits available options to user based on the details of a particular case/patient. The trigger for such limitations can vary based on the particular pharmaceutical product being prescribed. Triggers can include, for example, age of patient, weight of the patient, adult/juvenile status of the patient, or the like. For example, when, based on the patient's profile and/or the diagnosis, the patient is determined to be a juvenile, the system requests additional information, such as the weight of the patient. The particular information can vary based on the particular pharmaceutical product being prescribed. In the exemplary embodiment, the system limits the prescription options available to the user based on the suggestions and/or requirements for prescribing the pharmaceutical product to juveniles. In other embodiments and/or for other pharmaceutical products, the system can warn the user without limiting the available prescription options, may not warn the user, and/or may suggest a prescription option without limiting other available options.

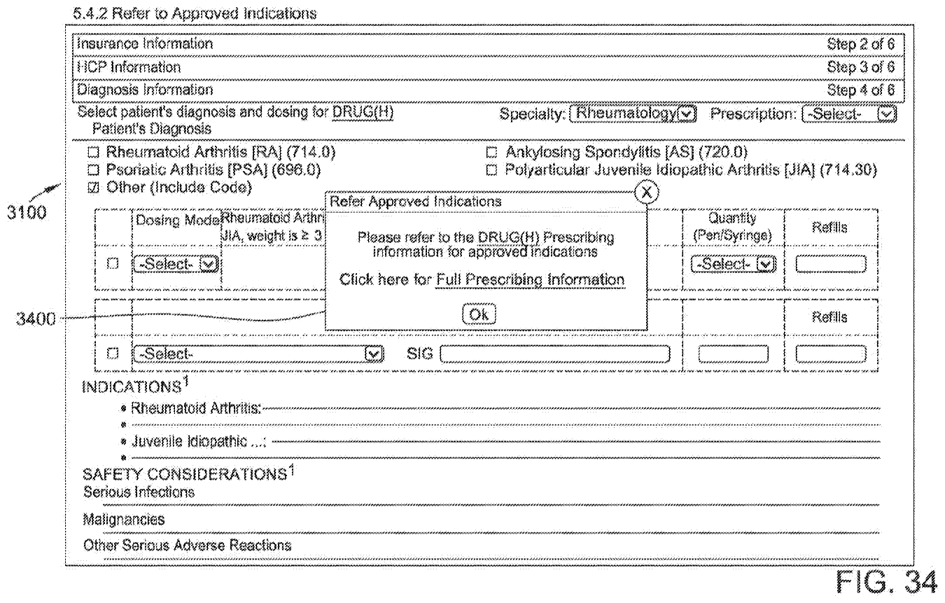

If desired, the system can permit the user to enter a diagnosis other than the listed diagnoses. As shown in FIG. 34, when the user selects “Other” as the diagnosis, a pop-up window 3400 is displayed over diagnosis window 3100 advising the user to refer to the drug's prescribing information for approved indications. In the exemplary embodiment, after closing pop-up window 3400, the user can enter an “other” diagnosis and proceed. In other embodiments, the system can prohibit entry of a diagnosis other than as listed in diagnosis window 3100. Similarly, when the user selects to enter a dosing other than one of the selectable dosing choices, a pop-up window (not shown) warns the user to refer to the drug's prescribing information for approved dosing. In the exemplary embodiment, after closing the pop-up window, the user can enter an “other” dosing and proceed. In other embodiments, the system can prohibit entry of a dosing other than as listed in diagnosis window 3100 or can not provide a warning to the user.

As embodied herein, in connection with some pharmaceutical products and/or specialties, there may be different indications, requirements, dosing, etc. depending on whether it is a new prescription for the product or a continuing prescription. For example, if the specialty is rheumatology, the system disclosed herein can provide a drop down to choose one of the following choices: New, Continuing. The system disclosed herein can provide an option for selection of the diagnosis for which prescription product is being prescribed. The diagnoses can vary depending on the specialty, pharmaceutical product, etc. For the rheumatology specialty, for example, embodiments of the system can provide an option to select the following diagnosis options using Multiselect Check Boxes: “Rheumatoid Arthritis (714.0)”; “Psoriatic Arthritis (696.0)”; “Polyarticular Juvenile Idiopathic Arthritis [JIA] (714.30)”; “Ankylosing Spondylitis (720.0)”; and “Other (include code):______”

Moreover, the patient's age can affect indications, prescribing requirements, dosing, etc. For example, the system can prevent the user from selecting any other diagnosis information if “Polyarticular Juvenile Idiopathic Arthritis [JIA] (714.30)” is selected. If the diagnosis is Polyarticular Juvenile Idiopathic Arthritis [JIA] (714.30), the system can prompt the user to enter patient's weight in pounds. If the diagnosis is Polyarticular JIA and the patient's weight is between 15 kg (33 lbs) to <30 kg (66 lbs) and the patient is 4 years of age or older, the dosing and frequency can be auto populated and not editable. The quantity and number of refills can be selectable by the user. If the user selects “Any Other Dosing” check box, the system can display a pop up with the warning message stating: “Please refer to the [Prescription Product Name] Prescribing Information for approved dosing regimens. Click here for full Prescribing Information.” Upon clicking of the full Prescribing information link, an external link to the full prescribing information can be opened in a new window. If the user selects OK in the pop up, the system can close the pop up and allow the user to enter any other dosing. The system can flag the referral as off label in database. If the user clicks on continue button, the system can save the referral. The system can check that the mandatory fields are filled in retain the referral status as “Patient Intake—In Progress”.

Additionally or alternatively, for example, if the user enters a weight 2: 30 kg (66 lbs) and the diagnosis is Rheumatoid Arthritis, Ankylosing Spondylitis, Psoriatic Arthritis, or Polyarticular JIA, the system disclosed herein can prompt the user to select a dosing mode. The system can provide a mandatory drop down with the applicable dosing modes. The available dosing modes can vary according to the particular drug being prescribed, and can include one or more of syringe, pen, tablet, liquid, or the like. After the dosing mode is selected, the system can auto populate the dosing and frequency. The fields can disallow editing by the user. The quantity and number of refills can be selectable by the user. If the user selects “Any Other Dosing” check box, the system can display a pop up with a warning message as described above, including an external link to the full prescription information, and can proceed as described above.

Additionally, as embodied herein, if the user selects other headers before completing the Diagnosis Information, the system can provide a pop up to the user to confirm the action. The pop up can read, for example, “Save Referral Information” with “Yes” and “No” buttons. If the user clicks the Yes button, the system can save the information entered by the user and retain the status of referral as “Patient Intake—In Progress”. If the user clicks the No button, the system can end the session without saving information.

FIG. 35 is a screenshot of a patient contact information window 3500 displayed after the user continues from diagnosis window 3100. The patient's phone number and alternate phone number are entered into patient contact information window 3500. In an exemplary embodiment, one or more of the phone numbers are prefilled based on patient information, such as the patient's profile, stored in the system.

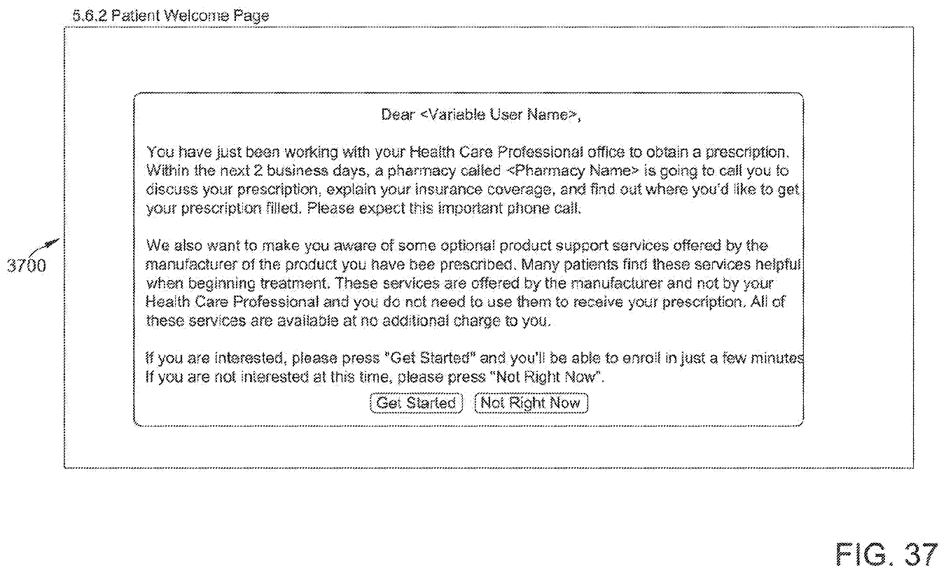

In the exemplary embodiment, the user can optionally use the system to display information concerning optional services related to the pharmaceutical product being prescribed after completion of the patient contact information window. In other embodiments, the optional services information can be presented at a different stage of the process. FIG. 36 shows a selection screen asking if the user would like to use subsequent screens to discuss optional service with the patient. Other embodiments proceed directly to FIG. 37 without presenting an option to the user regarding whether or not to discuss optional services with the patient, although the patient can decline to accept or consider any optional services. FIGS. 37-40 are screenshots of the optional services pages for review and completion by the patient. The time period while the patient completes and reviews the various optional services pages can be referred to as a patient interaction period. In the exemplary embodiment, during the patient interaction period, computing device 502 (shown in FIG. 5) enters a second mode, also referred to as a patient interaction mode, in which computing device 502 is prohibited from displaying data entered by the HCP. Accordingly, patients are prevented from viewing confidential and/or medical information entered by the HCP.

When the user selects, in FIG. 36, to discuss the optional services with the patient, a welcome page 3700 (shown in FIG. 37) is displayed for the patient. Welcome page 3700 explains briefly about the purpose of the subsequent pages, i.e., to offer optional services to the patient, and allows the patient to select whether or not to get started reviewing and/or signing up for optional services.

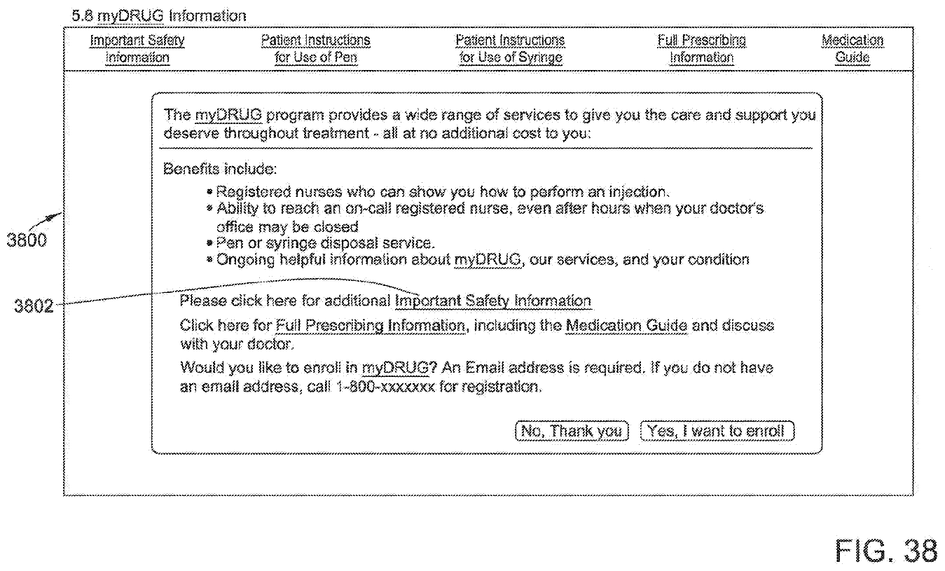

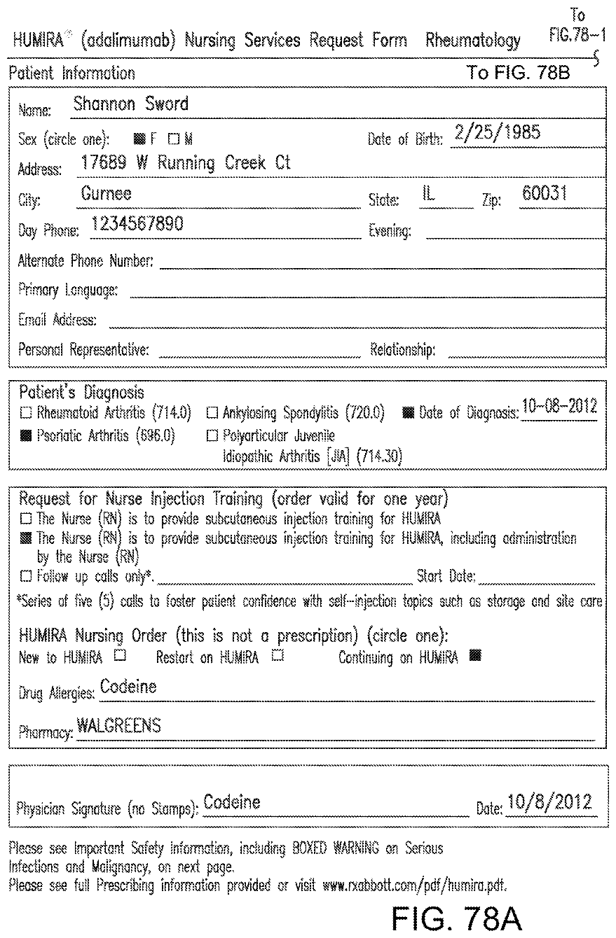

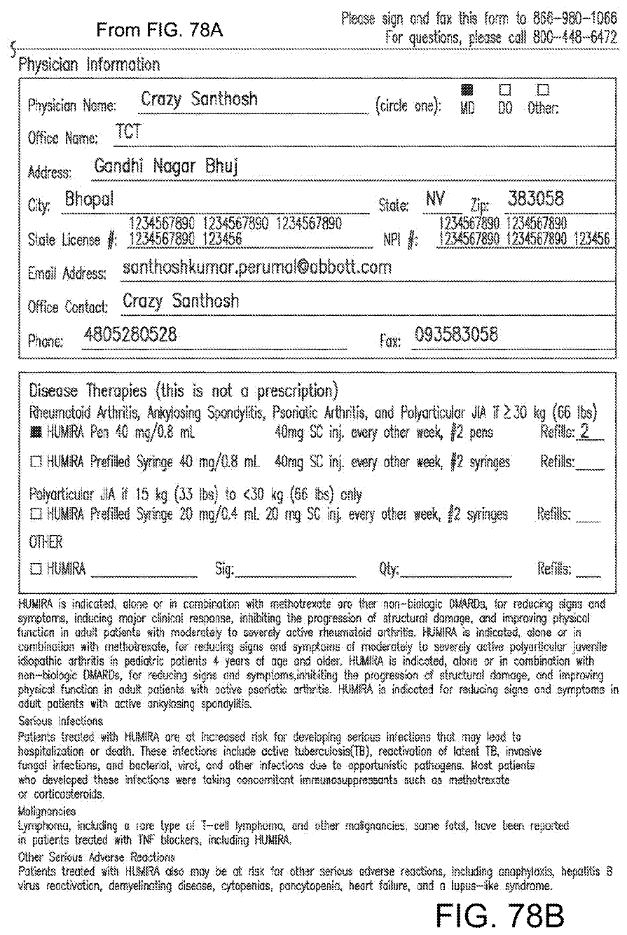

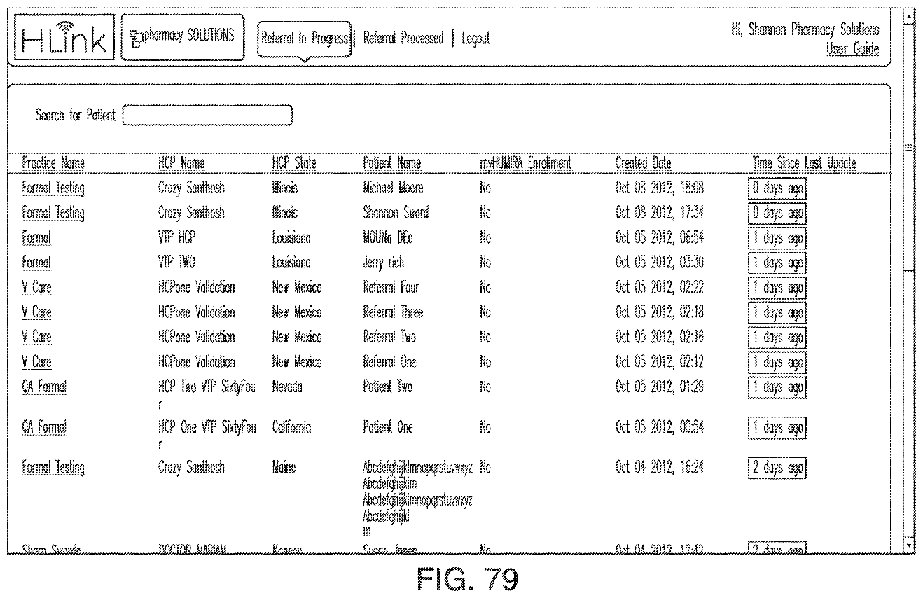

FIG. 38 is a screenshot of an exemplary information page 3800 about an optional support services program shown to the patient after selecting to get started on welcome page 3700. In other embodiments, other optional services can be presented, additionally or alternatively, to the patient. Information page 3800 includes the benefits that can be received from the support services program and provides links 3802 to additional information about the drug prescribed for the patient. The benefits can include, for example, training in the pharmaceutical product by registered nurses, disposal of syringes and/or other medical items, ongoing informational services, and after-hours access to a registered nurse for questions about the pharmaceutical product. For purposes of illustration and not limitation, FIGS. 78A and 78B illustrate an exemplary nurse injection training request form in accordance with an embodiment of the disclosed subject matter.

If the user elects to enroll in the support services program, consent pop-up 3900, shown in FIG. 39, is displayed over information page 3800. Consent pop-up 3900 provides the patient the ability to consent to or deny the disclosure of health information to the third parties providing the support service. For purposes of illustration and not limitation, FIG. 39B illustrates another exemplary consent pop-up in accordance with an embodiment of the disclosed subject matter. If the patient consents to the disclosure, the system displays a sign-up page 4000 for the support services program to the patient, as shown in FIG. 40. In the exemplary embodiment, at least some of patient information fields 4002 are pre-filled by the system based on the patient's profile information created as described above. Additional information not collected by the system, but needed for registration with the support services program is manually entered by the patient. If desired, the registration for the support service program occurs outside the system, such as through a website of the support services group. In such embodiments, the system can open the appropriate registration page in a separate window, program, tab, or the like, or can open the registration page within the system such that the registration page appears to be integrated within the system.

In the exemplary embodiment, the optional services are provided by a manufacturer of the pharmaceutical product prescribed for the patient. In other embodiments, services offered by one or more other third parties can be, additionally or alternatively, offered to the patient using the system.

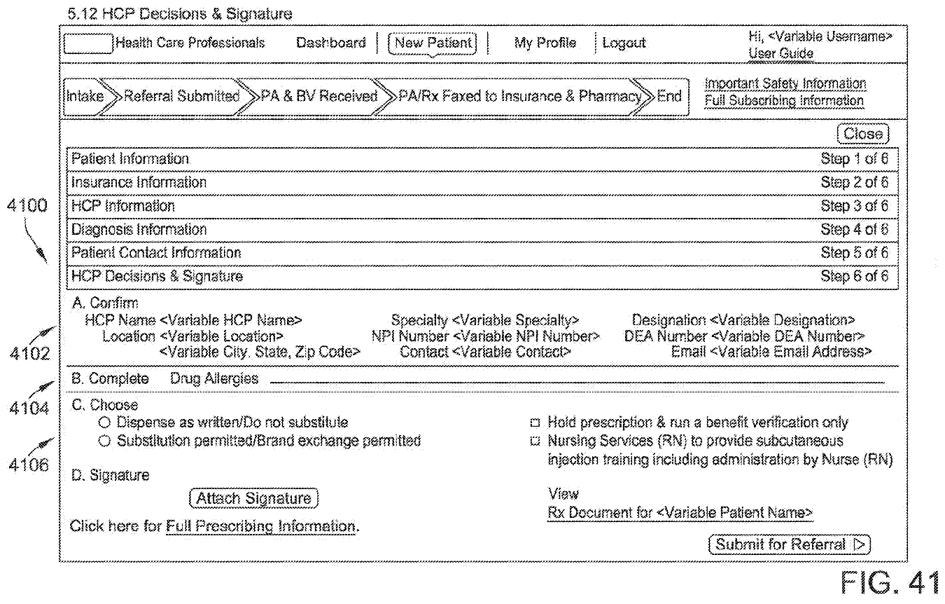

Following completion of registration for any desired optional service, or refusal to register for any such services, the intake process resumes with the HCP or office staff user. To continue the intake process, the HCP or office staff user indicates that the patient interaction period is complete. In the exemplary embodiment, the user is required to reenter his/her username and password to continue following the patient's review of the optional services. FIG. 41 is a screenshot of an HCP decision and signature window 4100 that is next displayed. The user confirms that certain information about the HCP in a confirmation section 4102 is correct. The user can also enter any known drug allergies of the patient into an allergy section 4104. In a handling section 4106, the user can select whether or not to allow substitution. Moreover, the user can select to have the prescription being created held, i.e., not filled, and only have a benefits verification run based on the prescription. Other optional services can also be selected in the handling section 4106. For example, in the exemplary embodiment, in which the pharmaceutical product is an injectable drug, the user can optionally request injection training of the patient by a registered nurse.

As described above, the patient intake procedure can include a number of discrete steps, partitioned into a set of screens for each step, including the general categories of “patient information,” “insurance information,” “HCP information,” “diagnosis information,” “patient contact information,” and “HCP decisions and signature.” Alternatively, the patient intake procedure can be grouped into a smaller number of discrete steps. For example, patient intake can be partitioned into the general categories of “patient information,” “insurance information,” “HCP information,” and “diagnosis information.” In this alternative embodiment, the four discrete steps can include acquisition of the same information as acquired in the embodiments described above. Moreover, as described in more detail below in connection with the generation of a medical order/prescription, in an exemplary embodiment a HCP signature need not be required upon patient intake.

As noted above, the prescription manager (including, for example and without limitation, various embodiments of the prescription manager, depicted in the figures as 10, 7310, and 112) can also manage the verification of benefits (31), either alone or in combination with one or more user devices (including, for example and without limitation, various embodiments of the user devices, depicted in the figures as 60, 500, 522, and 114). For example, a benefits verification request can be generated based upon information acquired during patient intake (21), including prescription information. The system can route the information to a “benefits verifier” in the form of a benefits verification request with or without a prescription referral. For example, in an exemplary embodiment benefits verification (31) can be preformed prior to generation of a prescription document or medical order document (41). In certain other embodiments, a medical order/prescription document can be generated prior to at least a portion of the benefits verification process, as described herein.

In an exemplary embodiment, the “benefits verifier” can be a “pharmacy receiver.” The pharmacy receiver can receive the information submitted by the HCP, including for example the benefits verification request. For example, in an exemplary embodiment, the pharmacy receiver can access the prescription manager via one or more user devices to receive the information submitted by the HCP. Alternatively, the prescription manager can transmit, for example via fax, secure email, or the like, the information to the pharmacy receiver. The pharmacy receiver can be, for example, a licensed pharmacy (whether or not the licensed pharmacy is the pharmacy that will fill the prescription), a pharmacy service company, a pharmacy support company, and/or pharmacy intermediary. The pharmacy receiver can verify benefits and, in an exemplary embodiment, identify any prior authorization (PA) requirements. The pharmacy receiver can electronically provide a benefits verification summary to the HCP (and, in an exemplary embodiment, the proper PA form required by the patient's insurer) via the medical treatment coordination system disclosed herein. The medical treatment coordination system can be configured to notify the HCP of the availability of the benefits verification and/or PA form. Such notification can be in the form of a preferential selection of an email notification, an SMS text notification, an icon, an alert, a phone call, or a change of status within the medical treatment coordination system. The PA form can be provided to the HCP by any suitable method of making the PA form available to the HCP. For example, the PA form can be made available by transmission from a computing device associated with the pharmacy receiver to a computing device associated with the HCP, by placing the PA form in the system and associating it with the patient, HCP, and/or incident, and/or by making the PA form available for download by the HCP. Moreover, as embodied herein, different entities can perform the tasks described herein. For example, one pharmacy receiver can perform the benefits verification, while a second entity can identify any needed PA.

As embodied herein, the pharmacy receiver can generate a benefits verification summary to summarize benefits provided to the patient by the patient's insurance provider. The summary can include, but is not limited to, deductible amount(s), co pay amounts, lifetime limits, whether three month supply prescriptions are covered and the applicable deductibles or co pay amounts, the availability of online and/or mail-order prescriptions, the insurance provider's preferred and/or mandatory pharmacy, duration of prior authorization, time period limitations for the prescription, pharmacy restrictions for filling the prescription, and/or other pertinent information related to the patient's insurance coverage. The information included in the benefits verification summary can vary based on the amount of information that the insurance provider will provide to the pharmacy receiver. In an exemplary embodiment, the pharmacy receiver generates a benefits verification summary if possible and transmits the benefits verification summary to the HCP or the patient. In other embodiments, a benefits verification summary may not be generated. Moreover, as embodied herein, different entities can perform the tasks described herein. For example, one pharmacy receiver can perform the benefits verification, while a second pharmacy receiver can identify any needed PA.

As further embodied herein, the HCP and/or the patient can be notified of the availability of the benefits verification summary and/or PA form, such as via an email notification, an SMS text notification, an icon, alert, phone call, or change of status within the system, or the like. With reference back to FIG. 22, as embodied herein, the status of the cases within open referral section 2206 indicates when a PA form and/or benefits verification (BV) summary have been received from the pharmacy receiver(s) in response to a submitted referral. Moreover, the pending action column of the open referral section 2206 for those cases for which a PA form has been received, but not yet completed, indicate the pending action is to fill out the PA form, as described in more detail below.

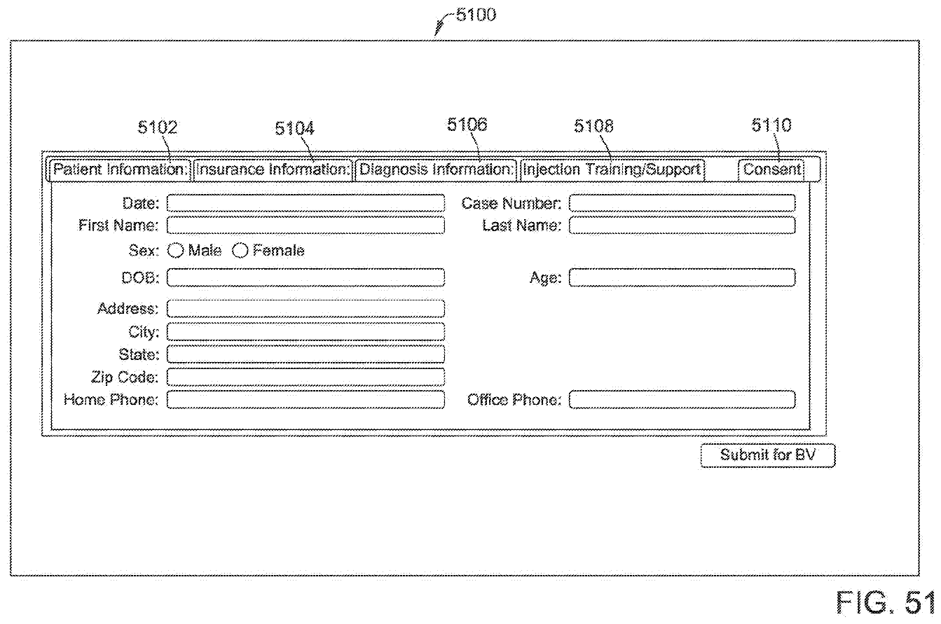

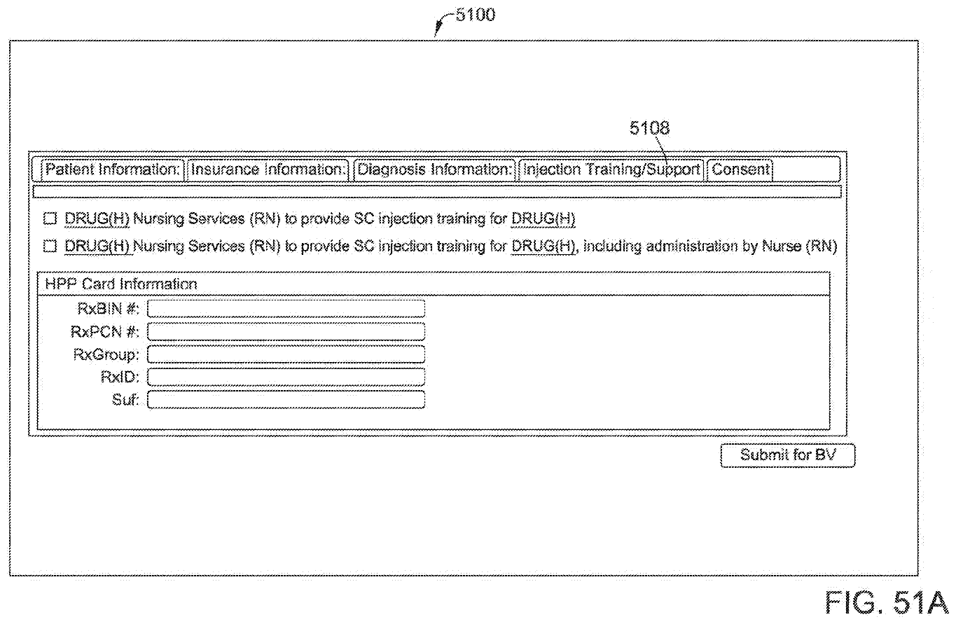

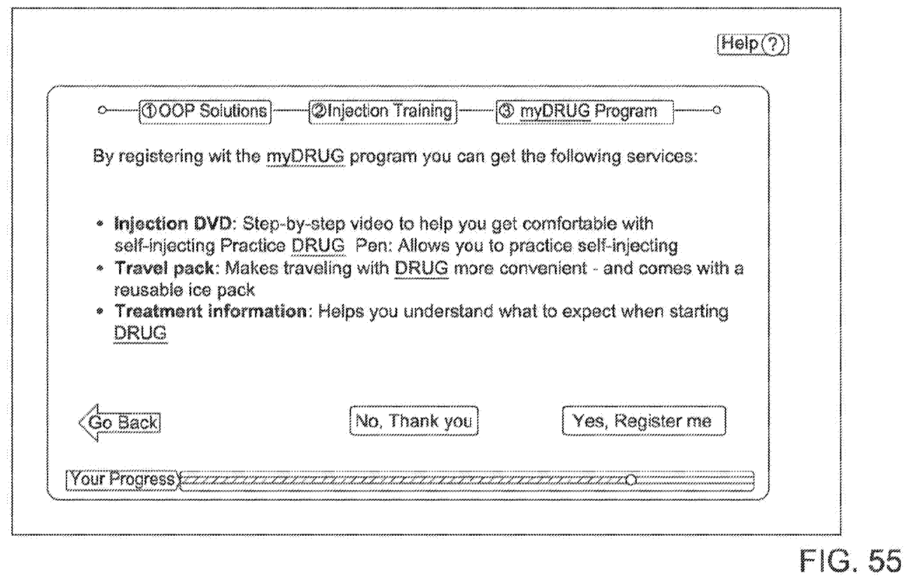

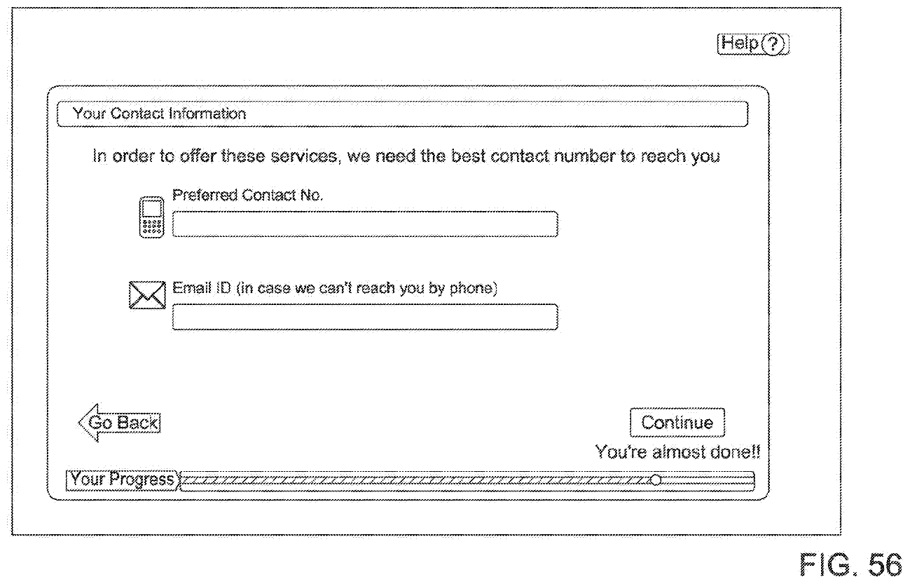

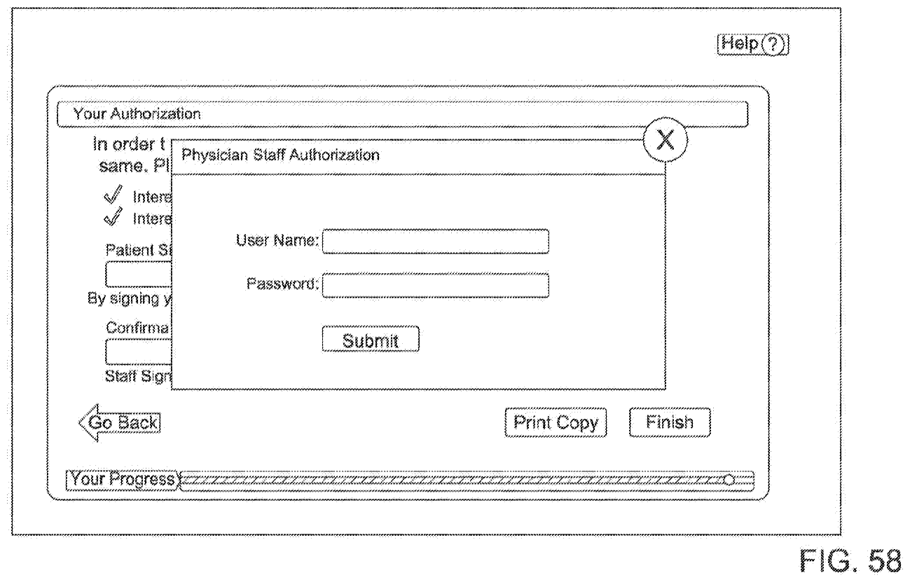

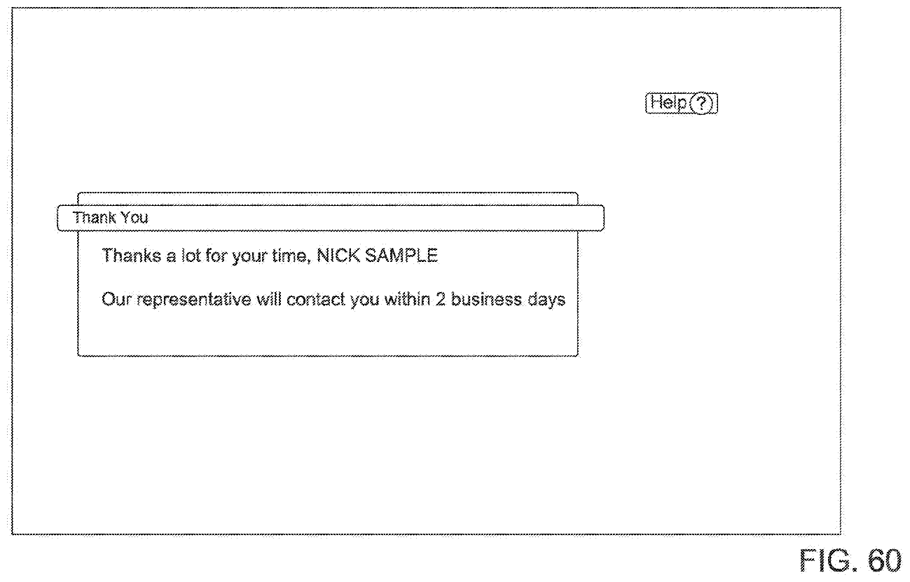

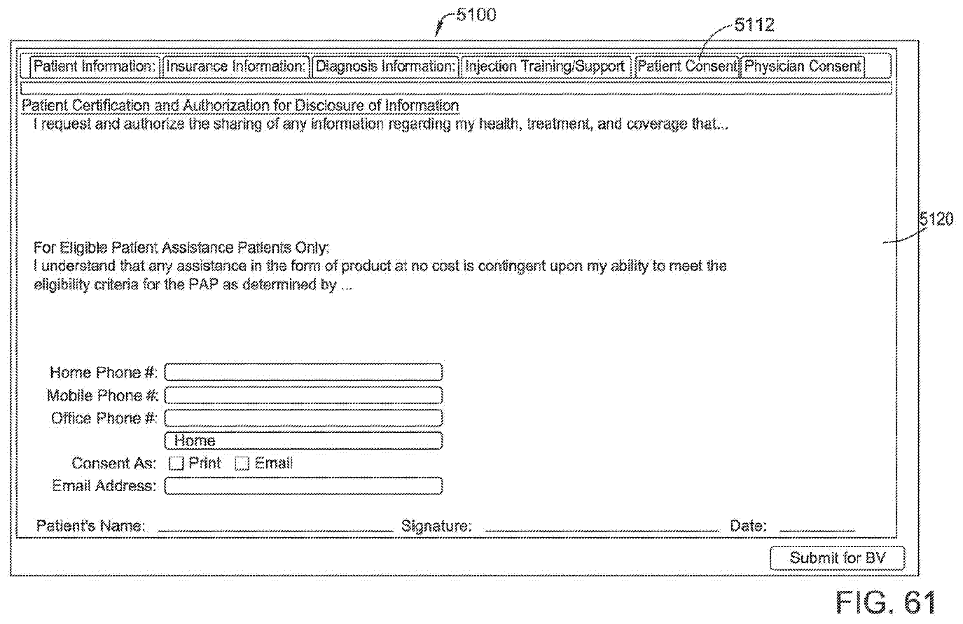

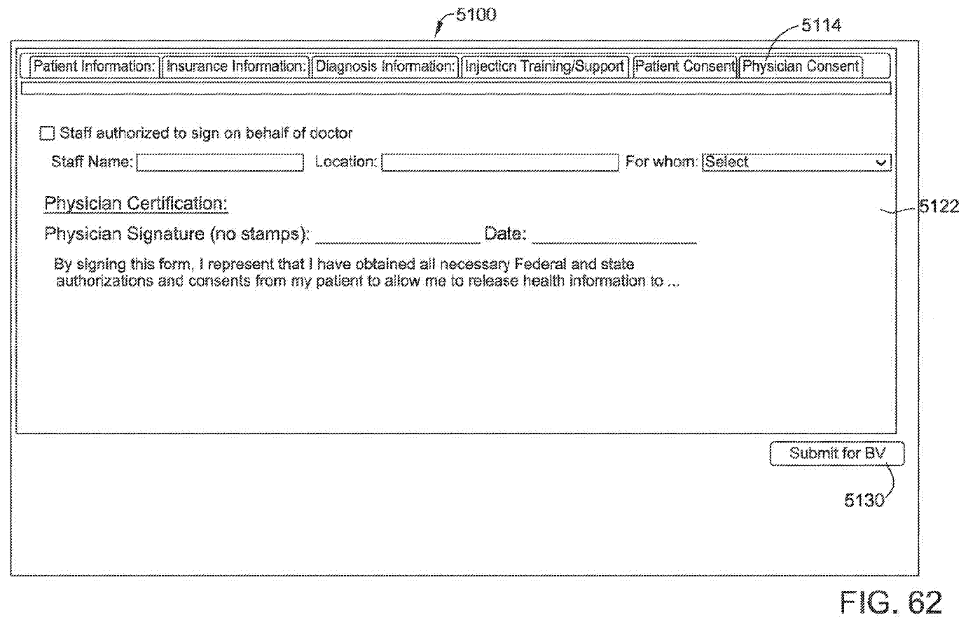

FIGS. 51-64 include screenshots of various exemplary tabs of a benefits verification request window 5100. Benefits verification request window 5100 can be presented to a user after the user registers a new patient, or after the user selects an existing patient. Benefits verification request window 5100 includes a patient information tab 5102, an insurance information tab 5104, a diagnosis information tab 5106, a training/support tab 5108, and a consent tab 5110. The consent tab 5110 can further include a patient consent tab 5112 and a physician consent tab 5114 as shown in FIGS. 61 and 62.

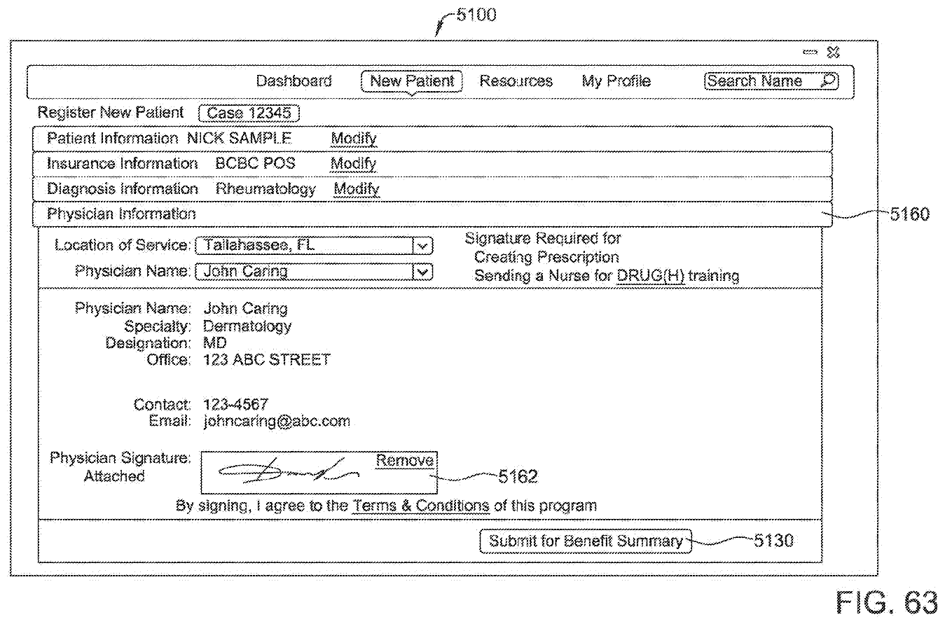

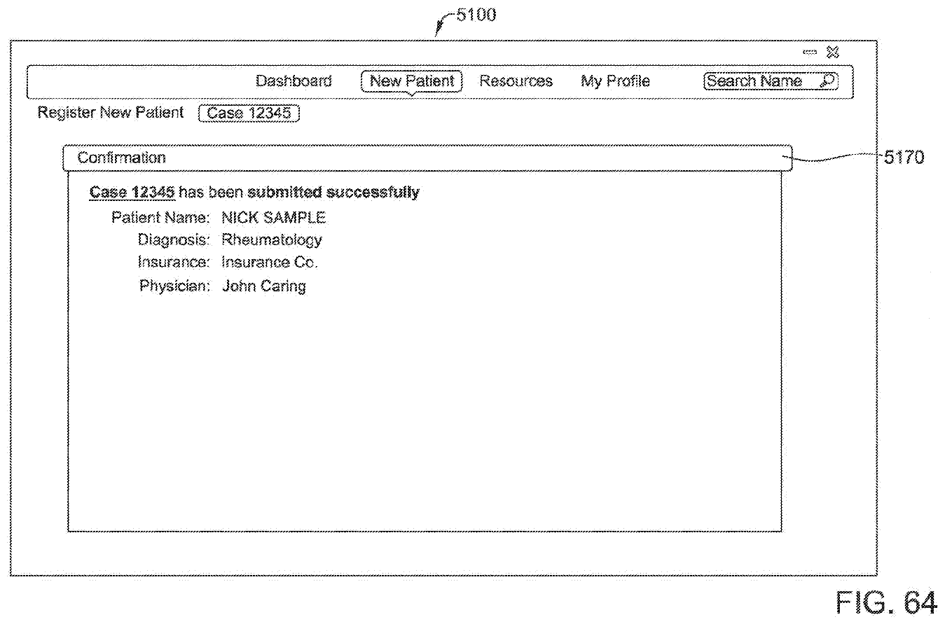

Once all information has been input into the benefits verification request window 5100, and the patient and physician have executed consent forms 5120 and 5122, the user can select a submission button 5130. Selection of submission button 5130 transmits a benefits verification request to the licensed pharmacy and/or equivalent provider. The benefits verification request includes the information from benefits verification request window 5100. As shown in FIG. 63, the benefits verification request window 5100 can also include a physician information tab 5160. Physician information tab includes physician information and a signature space 5162 for a physician signature, as well as submission button 5130. After submission button 5130 is selected, as shown in FIG. 64, a submission confirmation 5170 is displayed.

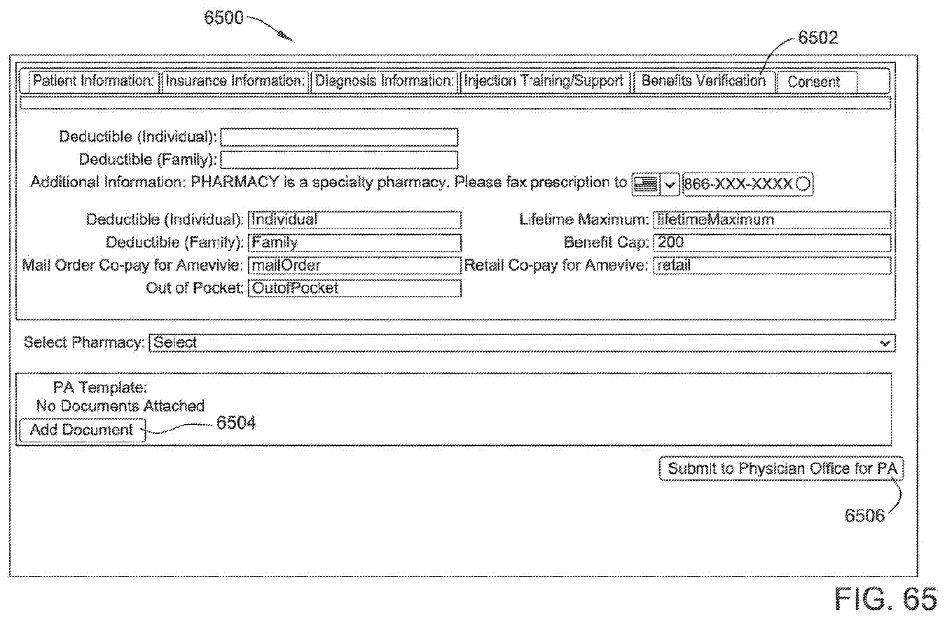

FIG. 65 is a screenshot of a benefits verification window 6500 as viewed by a pharmacy receiver upon receipt of the benefits verification request. A user can view and input benefits verification information (e.g., deductibles, out-of-pocket expenses, or the like) into a benefits verification tab 6502. By selecting an add document button 6504, the user can attach the appropriate prior authorization (PA) form for transmittal to the HCP. If desired and/or appropriate, the system disclosed herein can automatically identify and attach the appropriate PA form based on at least some of the information in the benefits verification request. Once the benefits verification information has been input and the appropriate PA form has been attached, a user selects a PA submission button 6506, and the PA form is transmitted to the HCP. In an exemplary embodiment, the PA form transmitted to the HCP is selected from a plurality of PA forms stored in a database, such as database 20 (shown in FIG. 1). Each PA form can be associated with a different insurance provider, as different insurance providers typically have different, distinct PA forms.

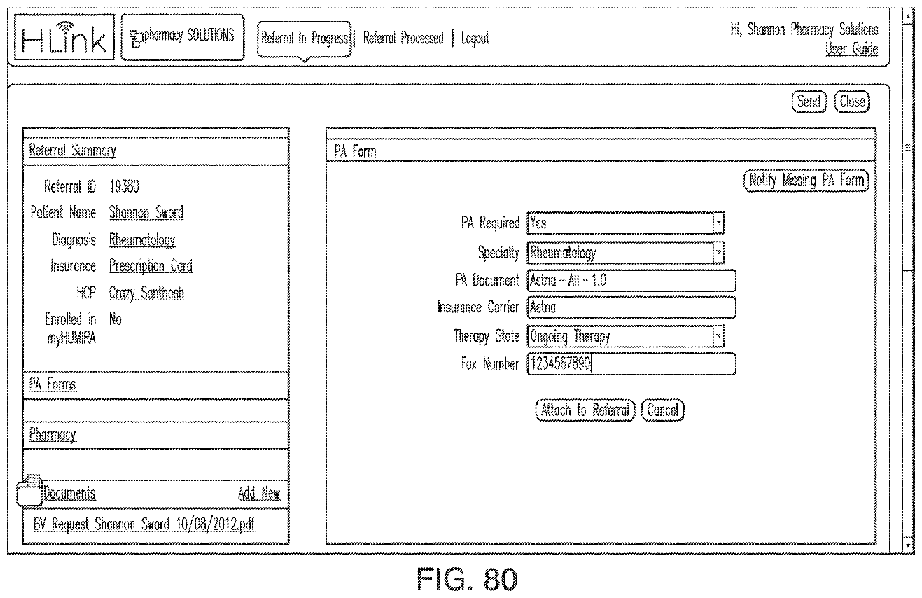

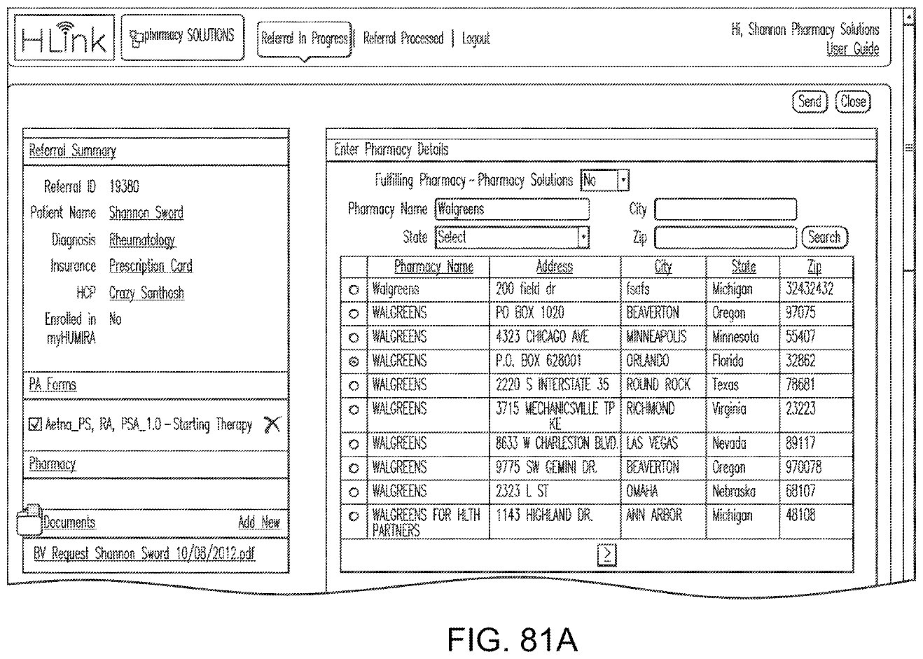

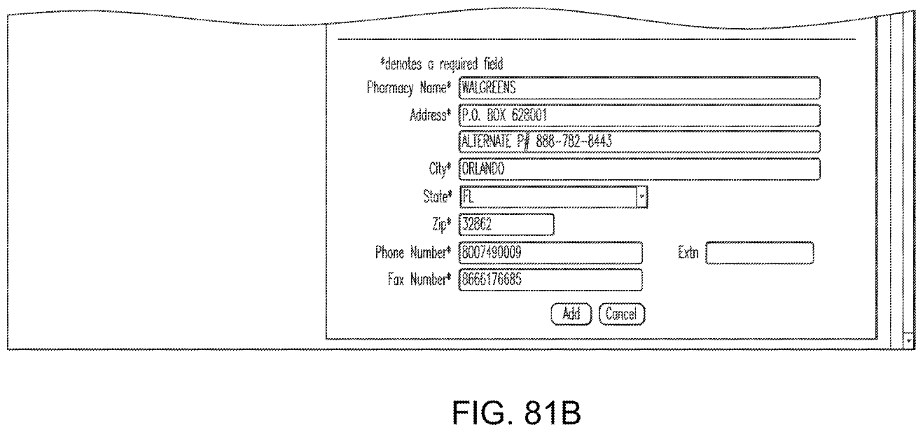

For purposes of illustration and not limitation, FIGS. 79-81B illustrate exemplary screens for a benefits verifier (e.g., pharmacy receiver) in connection with access by the benefits verifier to the system disclosed herein. FIG. 79 illustrates an exemplary dashboard screen in accordance with an embodiment of the disclosed subject matter. FIG. 80 illustrates an exemplary screen for selecting a PA form in accordance with an embodiment of the disclosed subject matter. FIGS. 81A and 81B illustrate an exemplary screen for entering pharmacy details in accordance with the disclosed subject matter.

As noted above, the prescription manager (including, for example and without limitation, various embodiments of the prescription manager, depicted in the figures as 10, 7310, and 112) can manage the selection, population, and release of certain predetermined forms, such as prior authorization forms, for a patient (51), either alone or in combination with one or more user devices (including, for example and without limitation, various embodiments of the user devices, depicted in the figures as 60, 500, 522, and 114).

As disclosed herein, for illustration, upon receiving the information from user device 7340, prescription manager 7310 can optionally decrypt the information. At 7421, prescription manager 7310 can select one or more insurance authorization forms for the prescription product or service, as well as determine the procedures to be followed, as required by the insurance provider(s), to obtain a prior authorization. Sometimes, different insurance providers or healthcare providers use different authorization forms and/or procedures for different prescription products or services. Prescription manager 7310 can select, from the authorization forms stored with prescription manager 7310, an appropriate authorization form for a specific prescription product or service based on, for example, the identity of the healthcare provider proscribing the product or service, the facility (e.g., hospital or clinic) with which the healthcare provider is affiliated, the type of product or service prescribed to the patient, or the insurance provider of the patient. As disclosed herein, for illustration, prescription manager 7310 can determine some of the information needed from the information sent by user device 7340 at 7413. For example, the identities of the healthcare provider and the patient and the type of product or service prescribed to the patient can be extracted from the information received from user device 7340 at 7413. In addition, prescription manager 7310 can retrieve some of the information needed from the information stored in the user account of the healthcare provider or the patient. For example, the facility with which the healthcare provider is affiliated can be retrieved from the healthcare provider's account or from the information sent by user device 7340 at 7413. The patient's insurance provider can be retrieved from the patient's account determined based on the patient's identify.

If the patient has multiple insurance providers, each insurance provider can require its own authorization form and/or procedure. As embodied herein, prescription manager 7310 can select, from the authorization forms stored with prescription manager 7310, multiple insurance authorization forms (e.g., one for each insurance provider of the patient).

Each insurance authorization form can include any number of fields, each field corresponding to a different piece of information that needs to be filled. As disclosed herein, for illustration, at 7423, for each authorization form selected for the prescription product or service, prescription manager 7310 can automatically fill in the required fields in the form based on the information available to prescription manager 7310, which can include information from the healthcare provider's user account and the patient's user account with prescription manager 7310, information received from user device 7340, information provided by the manufacturer or seller of the prescribed product, or information provided by the prescription service provider. In addition, if prescription manager 7310 has access to an electronic medical records system, prescription manager 7310 can retrieve relevant information (e.g., the patient's record) from the electronic medical records system.

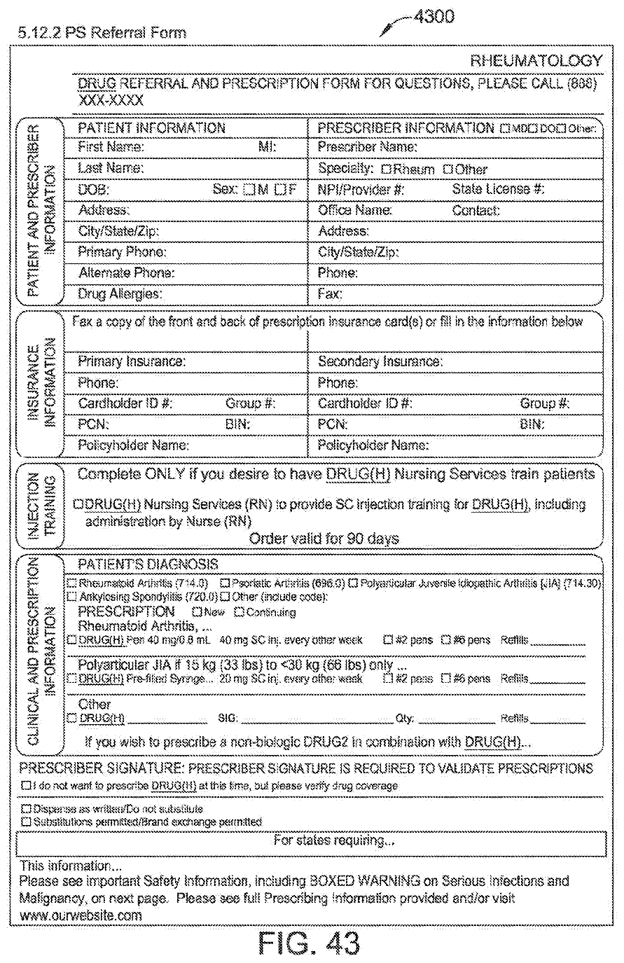

FIG. 43 illustrates a page of an example insurance authorization form. For example, the form can include a section for the patient's information, a section for the healthcare provider's (i.e., the prescriber's) information, a section for the patient's insurance information, and two sections relating to the prescribed product or service. Each section can include a number of fields. For example, under the “Patient Information” section, there are fields corresponding to the patent's first name, middle initial, last name, date of birth, gender, address, phone numbers, and drug allergies. Under the “Insurance Information” section, there are fields corresponding to the patient's primary insurance and secondary information, such as phone number, card-holder identification number, group number, policy holder name, or the like. Information needed for filling these fields can be retrieved from a data store (e.g., data store 7312 and/or data store 7360), for example, from the patient's user account or the patient's record from an electronic medical records system or information received from user device 7340 at 7413. The information is then populated into the authorization form automatically by system 7300 (e.g., specifically by prescription manager 7310). In the instance of multiple authorization forms (e.g., corresponding to multiple insurance providers), prescription manager 7310 can automatically populate the appropriate fields (e.g., in each authorization form). In addition, there are fields relating to the healthcare provider (i.e., the prescriber), the patient's diagnosis, prescription drug, or the like. Information needed for filling these fields can be retrieved, for example, from the healthcare provider's user account or information received from user device 7340 at 7413 or information supplied by the manufacturer or seller of the drug.

As disclosed herein, for illustration, at 7425, once the insurance authorization form or forms have been filled out, prescription manager 7310 can, optionally, encrypt the form(s) (e.g., for patient's privacy protection), and send the completed form(s) to user device 7340 associated with the healthcare provider. As appropriate, the insurance authorization form(s) can be selected and to allow the completed form(s) can be sent back to user device 7340 in sufficient time to allow the healthcare provider to receive the completed form(s) while the patient is still consulting with the healthcare provider. In this case, the healthcare provider can, as appropriate, review the form(s) with the patient and sign them. Other times, the completed form(s) can be sent back to user device 7340 after (e.g., a few hours or within a day) the patient's consultation with the healthcare provider). Additionally, prescription manager 7310 can conduct a quality/spelling check to ensure that each authorization form is filled in completely and the information entered into the form is spelled properly or correctly. In 7416, an HCP can save the populated insurance authorization form, for example by clicking a “save” button, at which time the prior authorization form can be saved in data store 7312.

As disclosed herein, for illustration, at 7415, the completed forms can be presented to the healthcare provider on user device 7340 for review and signing. To review a completed insurance authorization form, the healthcare provider can log into his/her account with prescription manager 7310. All the pending insurance authorization forms (i.e., corresponding to products or services prescribed to various patients) can be found in the healthcare provider's account. The healthcare provider can select a specific insurance authorization forms for review and signing.

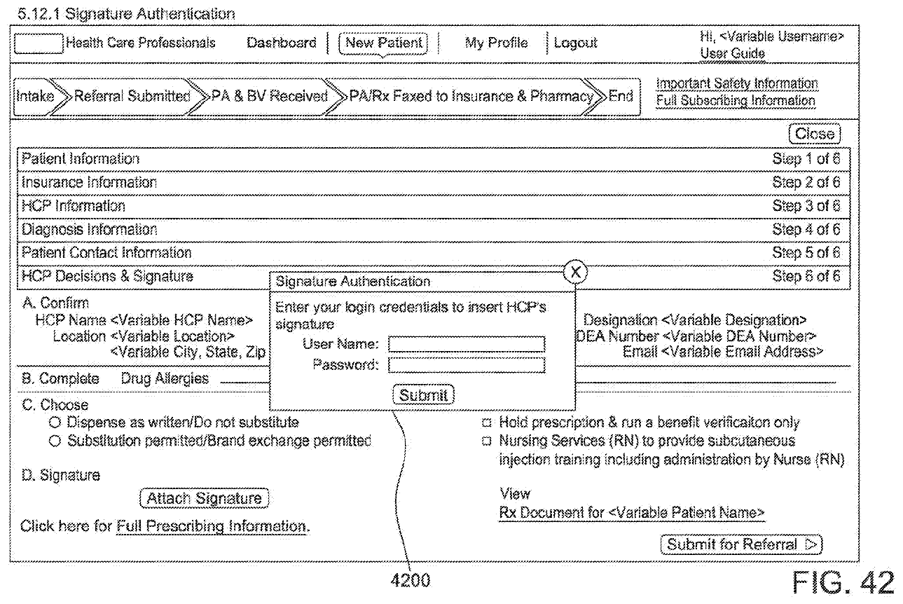

For purpose of illustration, there can be screens provided by prescription manager 7310, as part of its user interface, that guide the healthcare provider through the review and signing process. For example, FIG. 42 illustrates an example screen from which the healthcare provider can enter a user name and password in order to insert an electronic signature (e.g., stored with the healthcare provider's account or on user device 7340) into a completed insurance authorization form. Sometimes, a jurisdiction (e.g., a state) does not allow electronic signatures to be used on insurance authorization forms. In such cases, the healthcare provider can need to print a physical copy of the form and sign it on paper.

As disclosed herein, for illustration, at 7417, the healthcare provider can send a signed insurance authorization form to a prescription product seller 7320 (e.g., a pharmacy selected by the patient) or a prescription service provider 7330. Optionally, the healthcare provider can send other relevant documents, such as the patient's chart, along with the signed insurance authorization form. The form and optionally, the additional documents, can be sent using any applicable means, such as via fax or email.

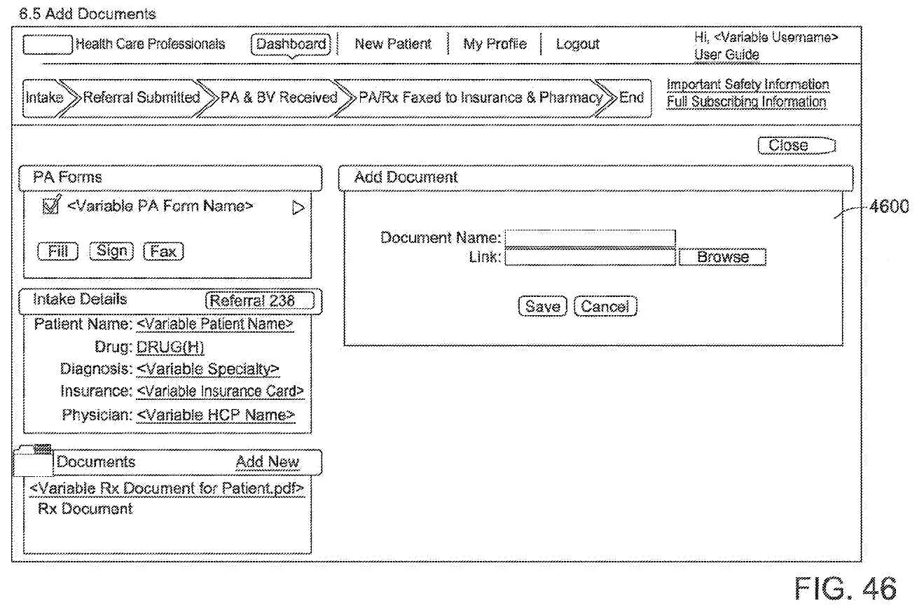

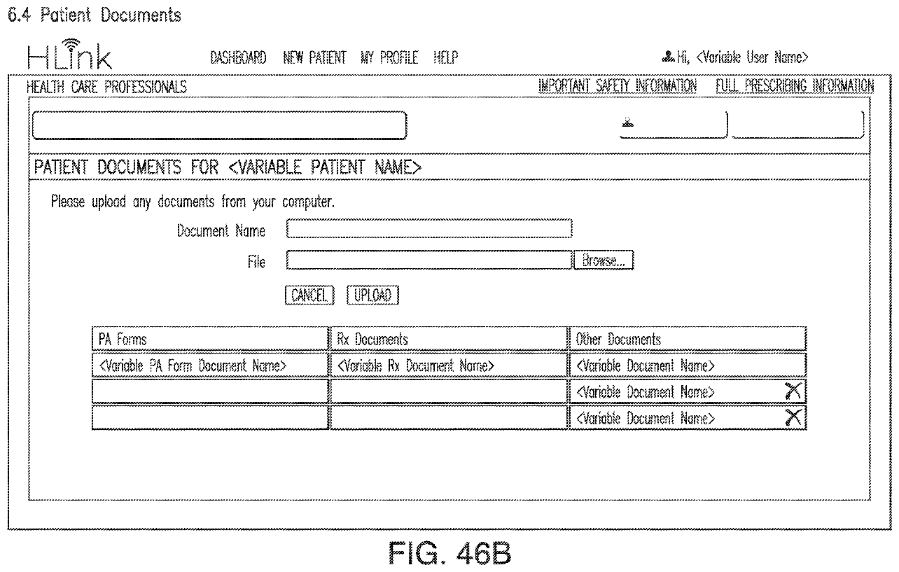

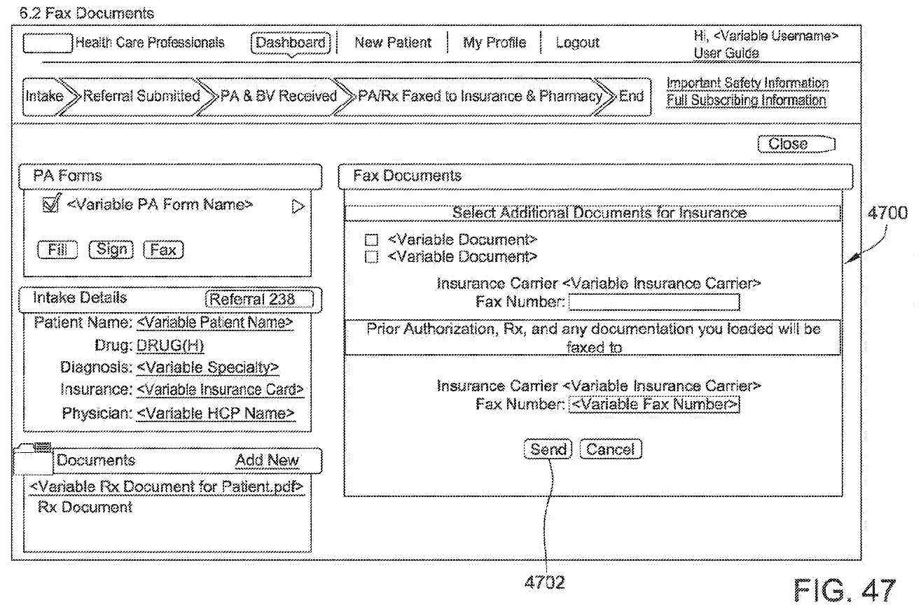

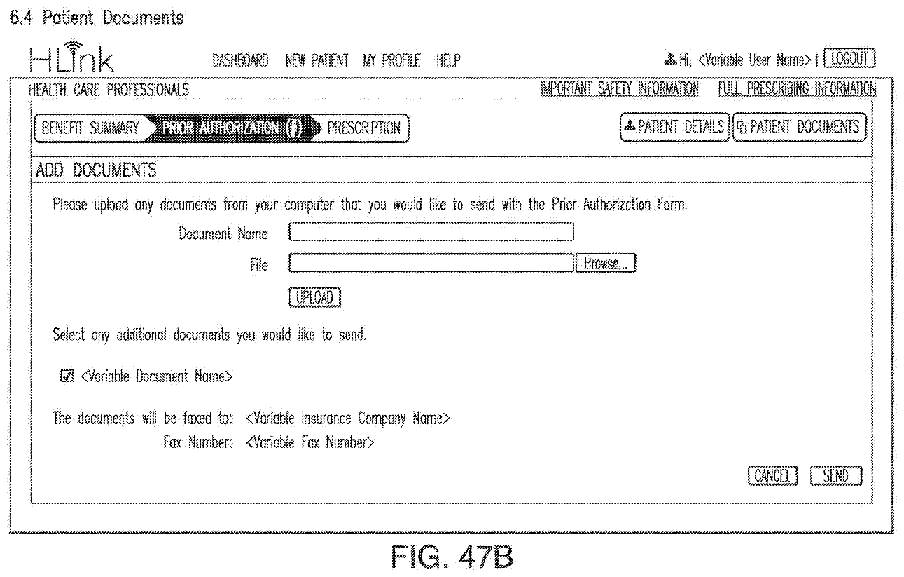

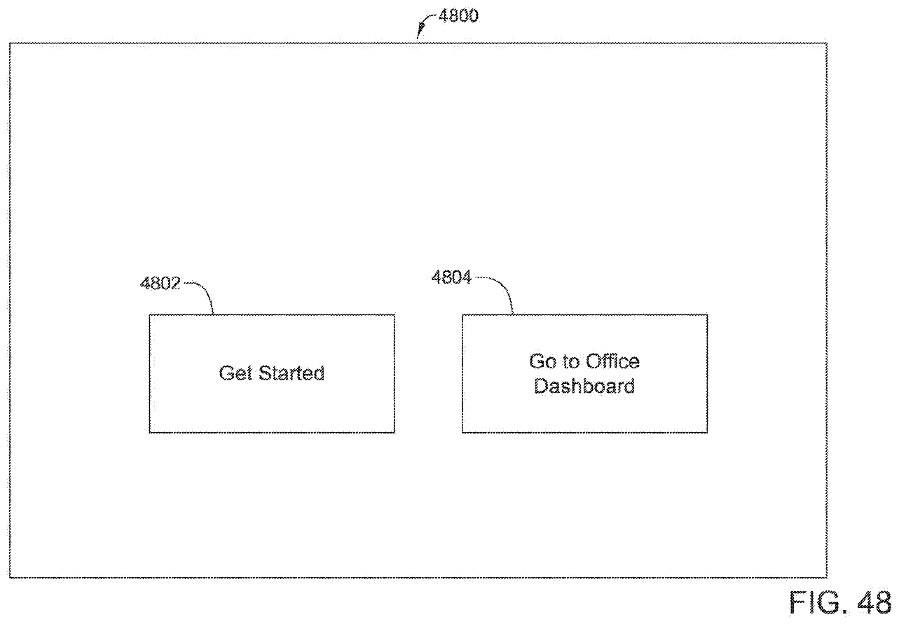

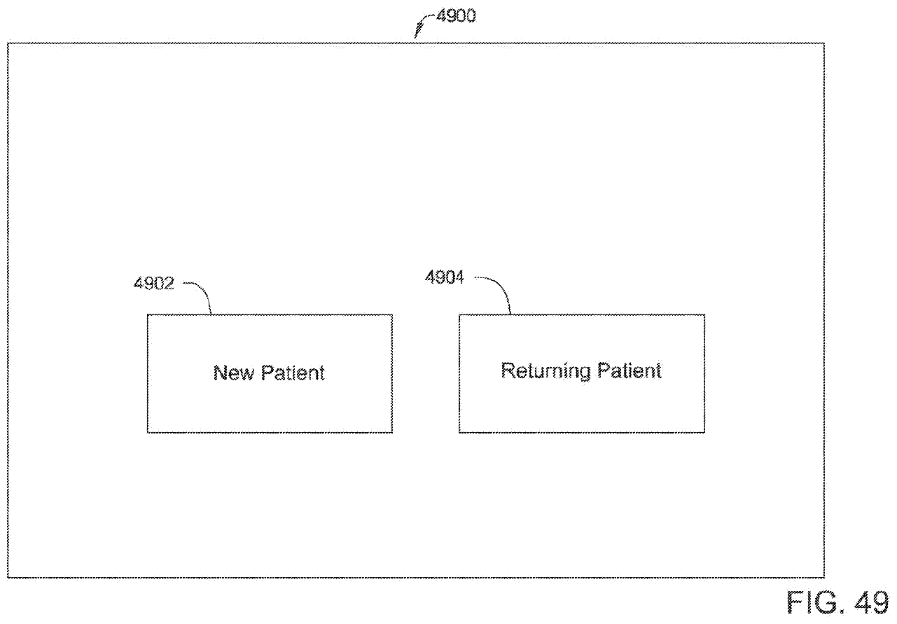

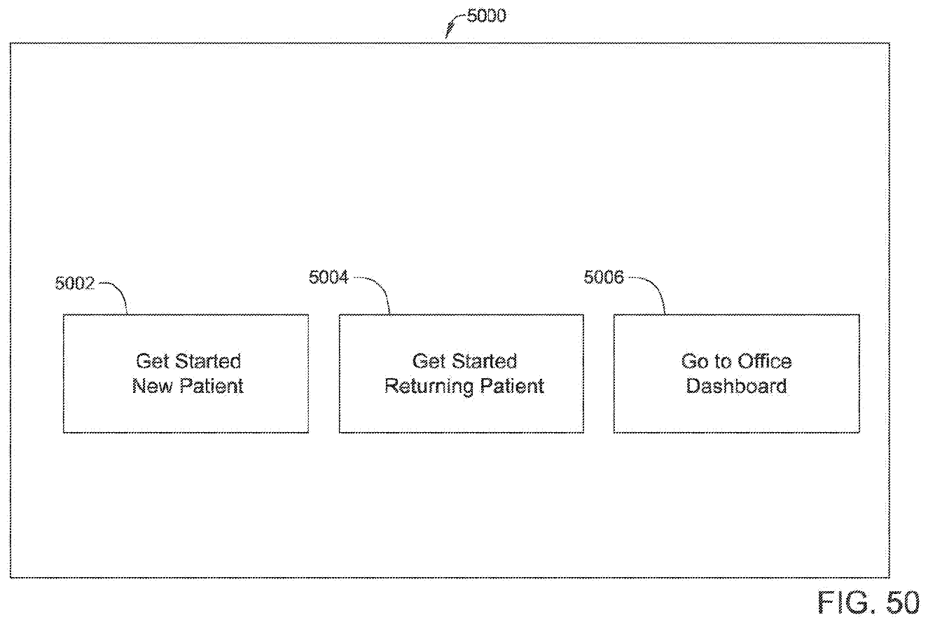

For purpose of illustration, screens can be provided by prescription manager 7310, as part of its user interface, to guide the healthcare provider to send a signed insurance authorization form to an appropriate recipient. FIGS. 46-47 illustrate example screens that guide the healthcare provider to fax a signed insurance authorization form. For example, from screen 4600 illustrated in FIG. 46, the healthcare provider can specify one or more additional documents, if needed, that can be sent together with the signed insurance authorization form. From screen 4700 illustrated in FIG. 47, the healthcare provider can enter a fax number of the recipient (e.g., a pharmacy or an insurance provider) for faxing the signed form and the additional documents.

For example, reference is made to the situation wherein the healthcare provider sends a completed and signed authorization form for a prescription medication to a pharmacy (i.e., a prescription product seller 7320) or an insurance provider that is a part of system 7300. As embodied herein, for illustration, the pharmacy can then forward the authorization form to the patient's insurance provider. If the patient's insurance provider approves the prescription medication, the insurance provider can notify the pharmacy of the approval. The pharmacy can then fill the prescription and contact the patient (e.g., telephone the patient using the phone number provided by the patient or notifying the patient through prescription manager 7310) so that the patient can pick up the medication at the pharmacy.

Sometimes, the patient's insurance provider can have a designated pharmacy that is not a part of system 7300 (i.e., a prescription product seller 7380). However, in order for the patient to receive payment benefit from the insurance provider, the patient can be required to obtain the actual medication from the insurance provider's designated pharmacy. In this case, even though the insurance provider has received the prescription and/or the authorization form from one pharmacy or insurance provider that is a part of system 7300 (i.e., a prescription product seller 7320), the patient's insurance provider can send the approval to another pharmacy that is not a part of system 7300 (e.g., its own designated pharmacy). The insurance provider's designated pharmacy can then fill the prescription and notify the patient. If the patient wishes for his/her insurance provider to pay for the medication, the patient can be required to pick the medication up at the insurance provider's designated pharmacy. Of course, the patient always has the option of paying for the prescription himself/herself, in which case the patient can be free to choose from which pharmacy to purchase the medication.

In an exemplary embodiment prior authorization (51) can be preformed prior to generation of a prescription document or medical order document (41). In certain other embodiments, a medical order/prescription document can be generated prior to at least a portion of the prior authorization process, as described herein.

Moreover, in an exemplary embodiment, one or more processors of the prescription manager (e.g., prescription manager 10 or 7310) can be configured to automatically select an appropriate prior authorization form, as described above. Alternatively, in an exemplary embodiment, the prior authorization form can be selected by the benefits verifier (such as, e.g., a pharmacy receiver) using the prescription manager 10. In an exemplary embodiment, the selection of the prior authorization form by the benefits verifier can be guided by a series of screens, as disclosed herein. For example, the benefits verifier can log into the system 7300, and the prescription manager can provide a user interface for the selection of a prior authorization form based on the patient provider information.

For example, and not limitation, after a referral and a prescription form is submitted to the pharmacy receiver, the pharmacy receiver can verify the patient's insurance benefits and identifies any prior authorization (PA) requirements. If desired, the pharmacy receiver prepares and submits a test claim for the prescription to the patient's insurance provider. The test claim can include the completed prescription form and a request to adjudicate payment for the prescribed product. If the claim is denied, the pharmacy receiver determines why the claim was denied. In particular, the pharmacy receiver determines if prior authorization from the insurance provider is required. If prior authorization is required, the pharmacy receiver determines the correct PA form for the patient's insurance provider. In other embodiments, the pharmacy receiver can directly contact the insurance provider, without submitting a test claim, to determine the insurance provider's claim requirements, the correct PA form (if applicable), the benefits provided by the insurance provider to the patient, or the like. In still other embodiments, data identifying the correct PA form(s) for particular insurance providers, benefits and requirements data concerning particular plans offered by insurance providers, or the like. can be used to automatically determine if a PA form is needed, which is the correct PA form, and/or benefits provided by the insurance provider to the patient. For example, the insurance provider can indicate the reason for the denial in a response to a test claim, and the PA form can be selected based on the reason for the denial.

If the correct form is already included in the system, the system automatically provides the correct PA form to the patient's HCP. In the exemplary embodiment, the PA form is an electronic PA form that includes a plurality of data fields. If the correct PA form is not included in the system, the pharmacy receiver prepares the PA form for inclusion in the system by, for example, creating a version of the form having the same document type as other forms in the system, e.g., a portable document format (PDF) document, and mapping the fields on the PA form to data available in the system to permit auto-population of the PA form by the system. The prepared PA form is then loaded into system by, for example, storing the PA form to a server such as server computer device 275. The PA form can be provided to the HCP by any suitable means for making the PA form available to the HCP. In the exemplary embodiment, the pharmacy receiver makes the correct PA form available to HCP via the system by associating the form with the particular patient and case to which it applies. In other embodiments, the pharmacy receiver can transmit the PA form to the HCP by making the form available for download by the HCP, by transmission to the HCP via electronic transmission, such as secure email or secure file transfer protocol (SFTP), or other transmission, such as via facsimile transmission.

As embodied herein, at least some PA forms that can be transmitted to the HCP include at least one electronically ‘tagged’ field that enables a processing device, such as computing device 502, to auto-populate the tagged field. The data used to auto-populate the PA form can include patient information (e.g., name, address, or the like), physician contact information (e.g., name, license number, or the like), information input into the benefits verification request window 5100 (shown in FIG. 51), office information (e.g., name, address), insurance information for the patient (e.g., company, plan number, or the like), and/or any other information pertinent to the fields of the PA form.

After the PA form is provided to the HCP (e.g., after the HCP accesses the PA form via the prescription manager), the HCP can manually populate the PA form with the required data, allow the system to automatically populate the form with the previously-provided information, or manually populate some portions of the PA form while allowing the system to automatically populate other portions of the PA form. The HCP can manually complete any fields that may not be auto-populated by the system, such as by providing additional medical information including lab results, previous treatments, or previous prescriptions. If required by the particular PA form, the HCP, and specifically the prescriber, signs the PA form electronically. In the example embodiment, the electronic signature is a digital representation of a physical signature by the HCP. The electronic signature can be captured at the time the physician is signing a particular PA form or may have been previously captured. If the electronic signature of the physician was previously captured, the physician can attach the existing electronic signature to the PA form to satisfy the requirement of signing the form. if desired, attachment of an existing electronic signature can require confirmation of the identity of the physician, such as via re-entry of a user ID and password. Moreover, if desired, one or more office staff members can be authorized to attach a physician's existing electronic signature to the PA form. Confirmation of the identity of the office staff member and his/her authorization to attach the signature can be confirmed prior to permitting the attachment.

The medical treatment coordination system then enables the HCP to submit the PA form to the insurer. The PA form can be electronically transmitted directly to a system maintained by the insurer, transmitted to a fax machine of the insurer, transmitted by secure email to the insurer, or transmitted to the insurer by any other appropriate method of transmission. In an exemplary embodiment, the PA form is submitted in a digital format. For example, the PA form can be submitted in an electronic PA (ePA) standard format. As mentioned above, the medical treatment coordination system presents optional services available to the patient for patient opt-in during the process. If the patient agrees to participate in one or more of these support services, third parties, including the drug manufacturer, that provide such services can proactively contact the patient to discuss financial assistance options, training, education, or support options. The contact can occur within hours of the initial engagement in the physician's office. The information to guide the discussion is transmitted, subject to the patient having opted-in, by the medical treatment coordination system to the service provider. In an exemplary embodiment, PA information typically included on the PA form is sent to the insurer in a format other than a form. Further, if desired, the PA form and/or PA information are sent using an electronic data interchange or a web service.

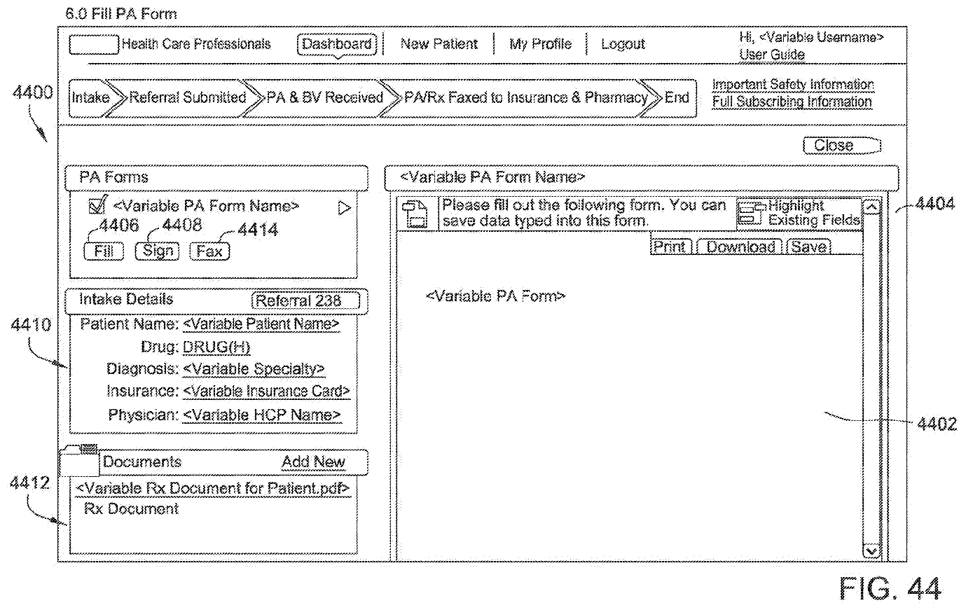

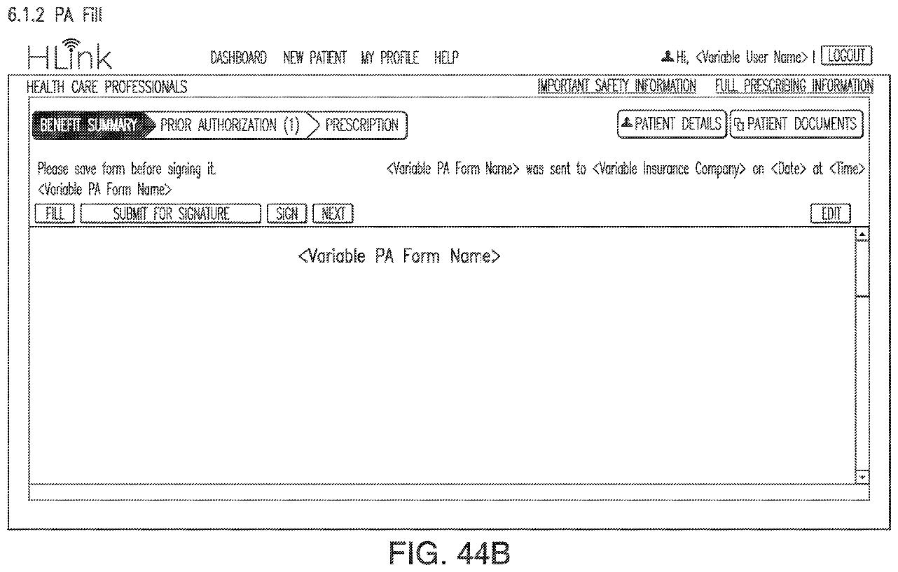

In an exemplary embodiment, and with reference to FIG. 44, by selecting to fill a PA form in dashboard window 2202, a PA window 4400 is opened, as shown in FIG. 44. PA window 4400 displays the PA form 4402 received from the pharmacy receiver in a PA form window 4404. PA form 4402 is a Tillable form, in which information can be entered by selecting a field, e.g., patient name, patient address, HCP name, HCP address, and diagnosis, and typing in the desired value for the field. In the exemplary embodiment, PA form can additionally or alternatively be filled by selecting a fill button 4406. If fill button 4406 is selected, the fields of PA form 4402 can be populated by the system disclosed herein with the appropriate information gathered in system 100 as described above. Specifically, the system can identify one or more data field in PA form 4402, maps corresponding data fields of stored patient and/or insurance data to the identified data fields, and populates the identified data fields with the stored patient and/or insurance data using the mapping. PA form 4402 can be partially completed by auto-populating the form and partially filled out manually, can be completely filled out automatically by the system, or can be completely filled out manually. In an exemplary embodiment, the system, or the PA form itself, can indicate PA form data fields where information is missing to alert the user that such data is required to complete the PA form. Further, in an exemplary embodiment, the system can prohibit saving and/or submitting a PA form until all required data fields have been completed. PA form 4402 can be signed by selecting, by the HCP or authorized staff member, a sign button 4408 and attachment of a signature as described above with reference to FIG. 41. In an exemplary embodiment, the HCP can create an electronic representation of the HCP's handwritten signature at the time of completion of PA form 4402 instead of attaching a previously stored electronic representation of the HCP's handwritten signature. For purposes of illustration and not limitation, FIG. 44B illustrates another exemplary PA screen in accordance with an embodiment of the disclosed subject matter.

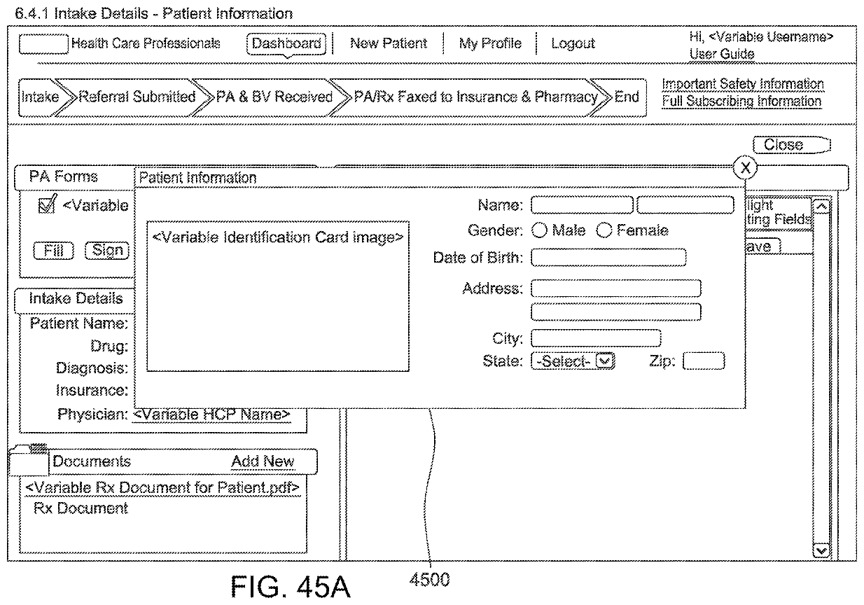

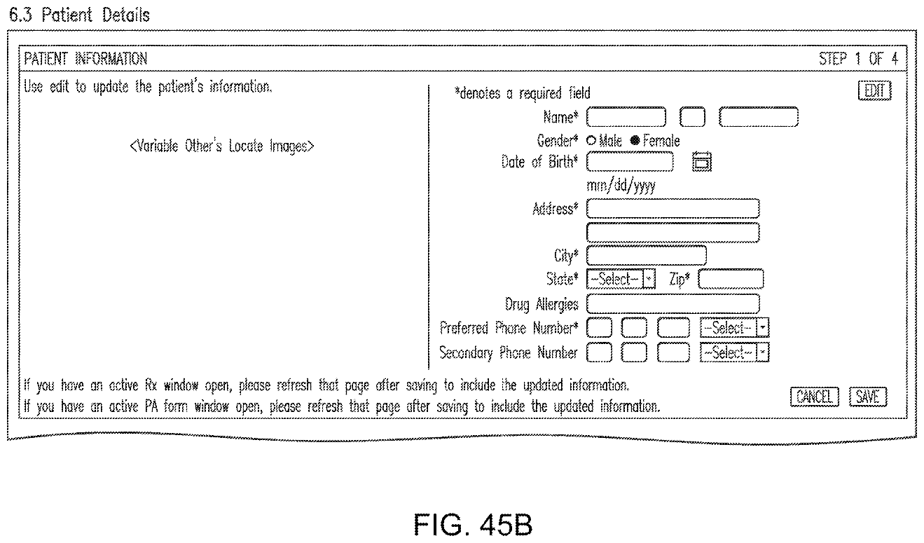

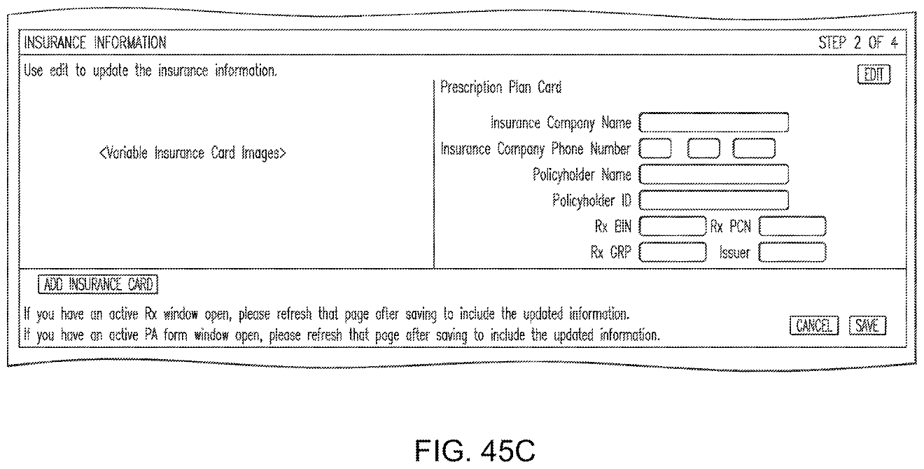

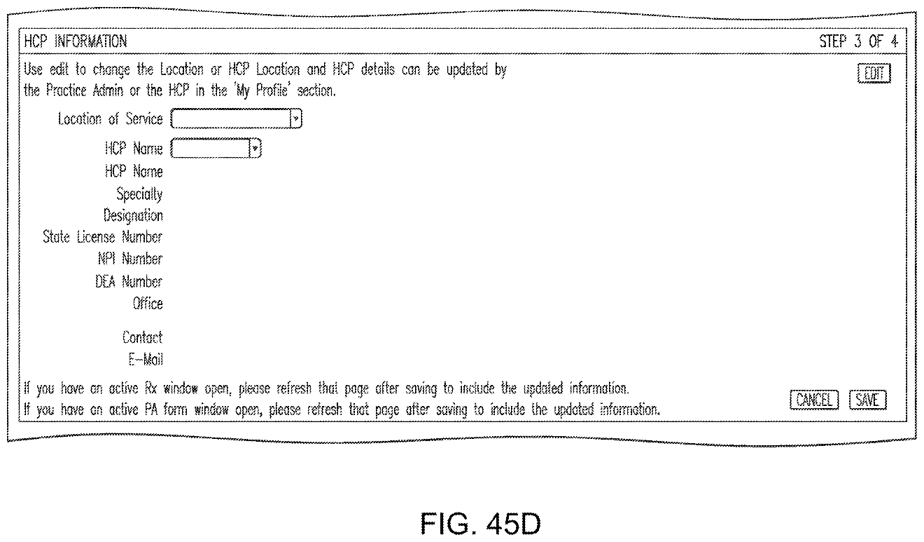

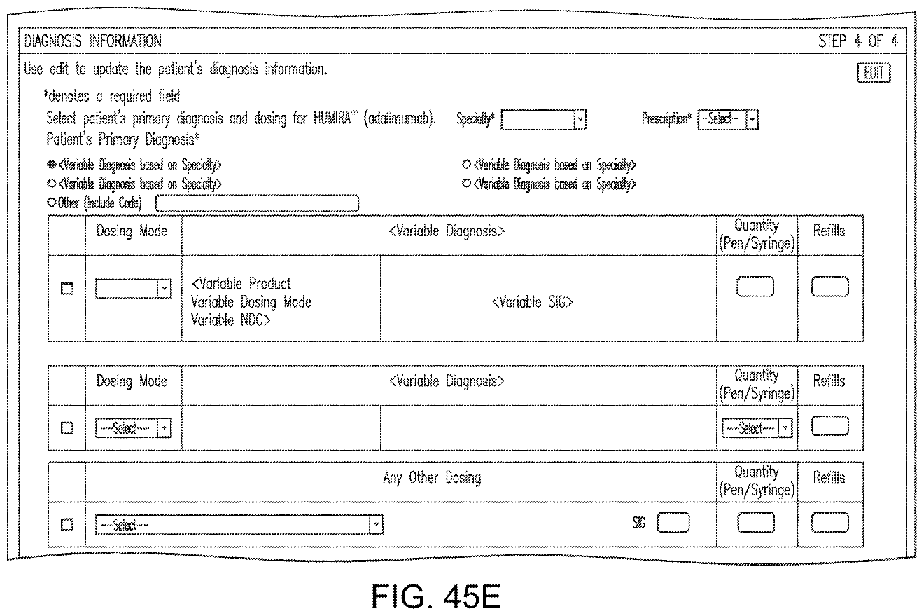

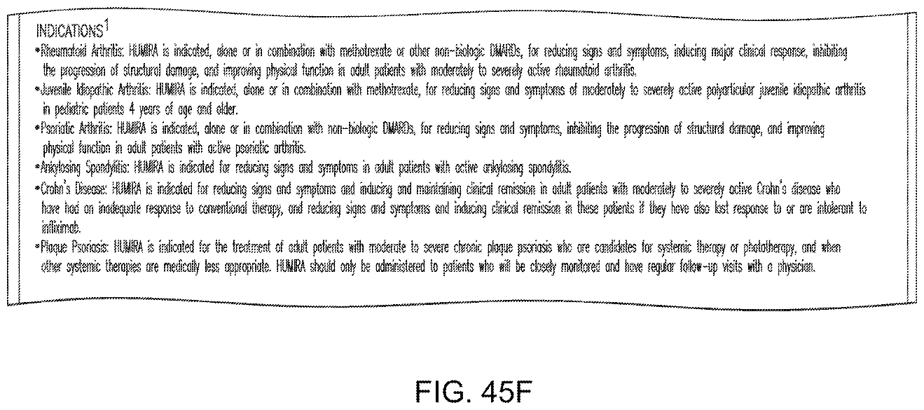

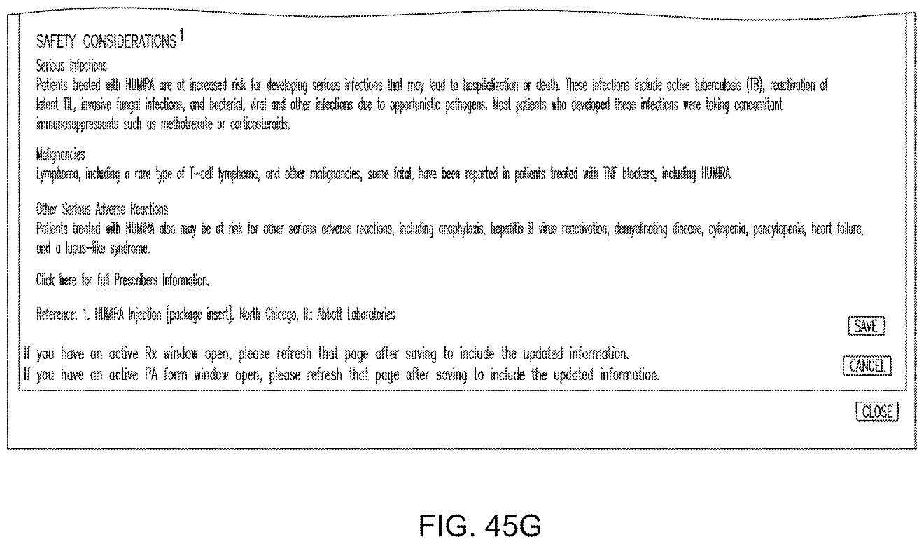

An intake details window 4410 displays a summary of information for the particular case including the patient name, the drug being prescribed, the diagnosis, the patient's insurance provider, and the HCP's name. Each of these items is selectable to view additional details. For example, if the user selects the patient's name, a patient information window 4500, shown in FIG. 45 is displayed over PA window 4400. Patient information window 4500 includes additional details about the patient, such as gender, date of birth, address, and the scanned image of the patient's identification document. For purposes of illustration and not limitation, FIGS. 45B-45G illustrate another exemplary patient information window in accordance with an embodiment of the disclosed subject matter.

If the user desires to submit additional supporting documents to the patient's insurance provider and/or the pharmacy that will fill the patient's prescription in addition to the PA form, the user can do so by selecting to add a new document to a documents window 4412. This selection opens a document addition window 4600, shown in FIG. 46, in which the user can locate additional documents, such as lab results, benefits verification summaries, additional notes and/or support for the diagnosis, etc. The selected documents are added to document window 4412 in preparation for submission along with PA form 4402. For purposes of illustration and not limitation, FIG. 46B illustrates another exemplary document addition window in accordance with an embodiment of the disclosed subject matter.

When the user is ready to send PA form 4402 to the insurance provider, the user selects a fax button 4414 (shown in FIG. 44), thereby opening a fax documents window 4700, shown in FIG. 47. For purposes of illustration and not limitation, FIG. 47B illustrates another exemplary fax documents window in accordance with an embodiment of the disclosed subject matter. In an exemplary embodiment, using contact information stored on the system and/or entered by the user, the PA form and associated documents are sent by facsimile transmission to the insurance provider and the patient's preferred or designated pharmacy for filling the prescription. In other embodiments, the PA form and documents are transmitted to one or both of the pharmacy and the insurance company by other means including, for example, via secure email, direct electronic transmission, printing for mailing, etc. If desired, PA information typically included on the PA form is sent to the insurer in a format other than a form. Further, if desired, the PA form, PA information, and/or additional documents are sent using an electronic data interchange or a web service. Moreover, if desired, information typically included in the additional documents (e.g., lab results, benefits verification summaries, additional notes and/or support for the diagnosis) is sent to the insurer in a format other than a document and/or sent using an electronic data exchange or a web service.

Additionally, the PA form and additional documents can be transmitted to an electronic medical record system for inclusion into the patient's record. Fax documents window 4700 displays the name of the patient's insurance provider and the insurance provider's fax number. In the exemplary embodiment, the user can select which documents to send to the insurance provider. All documents included in document window 4412 are displayed in fax documents window 4700 for selection for transmission to the insurance provider. In other embodiment, one or more documents can be preselected and/or mandatory for transmission to the insurance provider. Fax documents window 4700 also displays the name and fax number of the patient's preferred pharmacy for filling of the prescription. In the exemplary embodiment, all documents included in document window 4412, including the prescription and the PA form, are transmitted to the pharmacy. In other embodiments, one or more documents can be selectable for optional transmission to the pharmacy, including electronic prescriptions. When the user selects send button 4702, the selected documents are faxed by an exemplary embodiment of the system disclosed herein to the patient's insurance company at the fax number listed in fax documents window 4700, and all of the documents are also transmitted to the filling pharmacy at the listed fax number. In connection with an exemplary embodiment, when one or more documents are electronically transmitted (e.g., via fax) to either the benefits verifier (e.g., pharmacy receiver), payor (e.g., insurance provider), or prescription product seller (e.g., pharmacy), the documents can be identified as originating from the HCP practice, such as by including information about the practice or prescriber name, address, phone, and/or fax information. For example, when sent via fax, the practice name and fax number an appear on the header of the fax.

As noted above, in an exemplary embodiment, the prescription manager (including, for example and without limitation, various embodiments of the prescription manager, depicted in the figures as 10, 7310, and 112) can manage the generating of (41) and execution (61) of a medical order/prescription document for the prescription product for the patient, either alone or in combination with one or more user devices (including, for example and without limitation, various embodiments of the user devices, depicted in the figures as 60, 500, 522, and 114). For example, as disclosed herein, generation (41) of a medical order/prescription can include the generation of a medical order/prescription document for a prescription product based on at least a portion of the patient intake information and the medical order/prescription information. That is, for example, the medical order/prescription document can be generated based on a portion of the patient intake information and/or a portion of the prescription information, individually or collectively. In an exemplary embodiment, patient intake information can include information beyond what is required for generation of the prescription document, and as such, a subset of the patient intake information can be used.

Moreover, in an exemplary embodiment, generation (41) of the medical order/prescription can include generation of a prescription document (i.e., a document, signed by a physician, which can be used to acquire a prescription product from a prescription product seller, e.g., a pharmacy). Alternatively, generation (41) of the medical order/prescription can include generation of a medical order (i.e., an order by a healthcare provider for the provision, administration, execution, or the like of a medical service of administration of a medical product). For example, a medical order can be generated that provides for the administration of a medical product (which can, but need not, be a product for which a prescription would be necessary if a patient were to acquire it from a prescription product seller) in a facility of the healthcare provider.

In an exemplary embodiment, execution (61) of a medical order/prescription can include the transmission of a prescription document to a prescription product provider (e.g., a pharmacy), or to a provider of the patient (e.g., an insurance provider). Similarly, execution (61) of a medical order/prescription can include the transmission of a medical order document to a medical service provider, healthcare provider (e.g., for the administration of a medical product), prescription product provider (e.g., a pharmacy, for example where the prescription product does not require a prescription), and/or a provider of the patient (e.g., an insurance provider). Moreover, execution (61) of a medical order/prescription can include the administration of a medical product or the provision of a medical service. For example, execution of a medical order/prescription can include the injection of a biologic product. As noted above, as used herein the term “transmission” (or “transmit”) can include any means of electronic transmission, such as by fax, email, electronic access via one or more user devices, HTTPS transmission, or the like.

Description will now be made, for purposes of example and illustration and not limitation, of certain exemplary embodiments in accordance with the disclosed subject matter in connection with the generation of a prescription for a prescription product. However, one of ordinary skill in the art will appreciate that the description below can enable the generation and execution of a medical order in like manner, and as such, the presently disclosed subject matter is not to be limited to generation and transmission of a prescription product.

To complete a prescription for a prescription product, a signature of the HCP can be required. In an exemplary embodiment, the electronic signature of the HCP created previously (and described herein) can be attached to complete the prescription. In other embodiments, the HCP's signature can be attached by contemporaneously capturing an electronic representation of the HCP's handwritten signature. In an exemplary embodiment, if the logged-in user of the system disclosed herein is the HCP, the user can attach the HCP's electronic signature. In an exemplary embodiment, if the user is a staff member authorized to sign for the HCP, the user can attach the HCP's electronic signature. Upon selecting to attach the HCP's signature, the user can be required to reenter his/her login credentials, i.e. username and password. An authentication pop-up 4200, shown in FIG. 42, can be displayed over HCP decision and signature window 4100. If the user enters incorrect credentials or is not authorized to sign on behalf of the HCP, the user is prevented from attaching the HCP's signature. If the user enters correct login credentials and is authorized to sign on behalf of the HCP, the HCP's signature is attached and a complete prescription and referral is ready for submission to a pharmacy receiver.

From HCP decision and signature window 4100, the user can view and/or submit the referral and prescription form. FIG. 43 is a first page of an example referral and completed prescription form 4300. Although shown with all of its information fields empty in FIG. 43, in operation, prescription manager (such as, for example, prescription manager 10, 7310, 112) or alternatively user device (such as, for example, user device 60, 7340, or 500) can populate all fields, including attaching the HCP signature (if applicable), with the information generated and/or collected as described above. In an exemplary embodiment, referral and prescription form 4300 can include additional information such as the scanned images of the patient's insurance card(s). In other embodiments, referral and prescription form 4300 can include more or less information. Moreover, the particular format and information included in referral and prescription form 4300 can be varied based on, for example, the requirements and/or desired format of the pharmacy receiver to whom the referral and prescription form 4300 will be transmitted.

When, and if, the user selects to submit referral and prescription form 4300 to the pharmacy receiver, referral and prescription form 4300 can be transmitted to a pharmacy receiver. As embodied herein, for illustration, referral and prescription form 4300 can be transmitted electronically to pharmacy receiver via a network, such as via the Internet. In other embodiments, referral and prescription form 4300 can be transmitted by any suitable transmission including, for example, by facsimile transmission, attachment to a secure email transmission, electronic transmission via a wireless network, transmission via a local area network, faxed, or printed and mailed, or the like. In connection with an exemplary embodiment, when the prescription form is transmitted (e.g., via fax) to either the benefits verifier (e.g., pharmacy receiver), payor (e.g., insurance provider), or prescription product seller (e.g., pharmacy), the documents can be identified as originating from the HCP practice, such as by including information about the practice or prescriber name, address, phone, and/or fax information. For example, when sent via fax, the practice name and fax number an appear on the header of the fax.

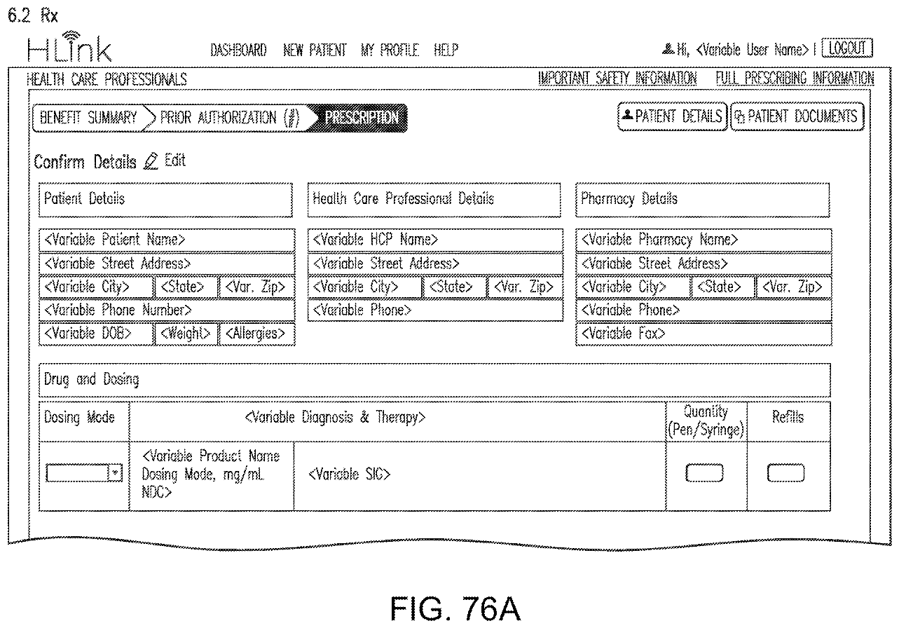

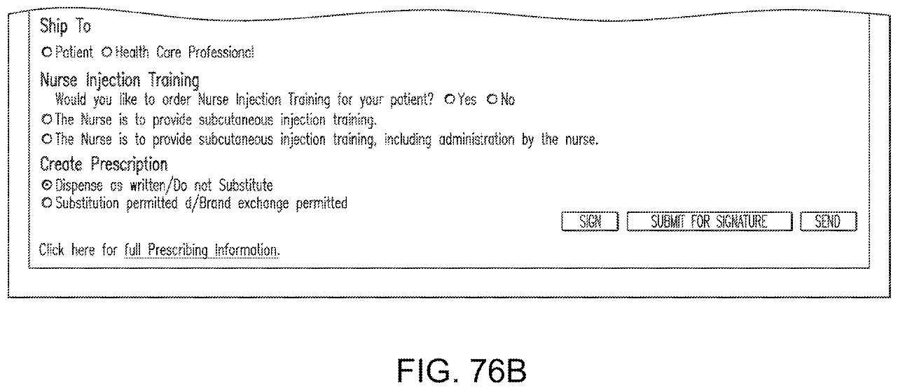

For purposes of illustration and not limitation, FIGS. 76A and 76B illustrate another prescription screen in accordance with an embodiment of the disclosed subject matter.

As disclosed herein, for illustration, prescription manager 7310 can implement and support additional functions that help healthcare providers and patients. For purpose of illustration, when the patient has received the prescribed product or service (e.g., a pharmacy has filled a prescription medication, which has been picked up by the patient or otherwise provided to the patient (e.g. mailed), or the patient has consulted with a specialist or received the prescribed treatment), a corresponding prescription product seller 7320 (e.g., the pharmacy) or a corresponding prescription service provider 7330 (e.g., the specialist) can indicate this information to prescription manager 7310 (e.g., accessing the corresponding website using an appropriate user device). Prescription manager 7310 can in tum update the information in the healthcare provider's user account so that the healthcare provider knows that the patient's prescription has been filled.

For purpose of illustration, if the healthcare provider has not signed a completed form for some time (e.g., a few days), prescription manager 7310 can send reminders to the healthcare provider to review and sign the form. The reminders can be in any applicable format. For example, prescription manager 7310 can send the healthcare provider reminders as emails, text messages, voice messages (e.g., through automated telephone calls), or the like. Some of these reminders do not require the healthcare provider to actually log into his/her account with prescription manager 7310 in order to receive them so that the healthcare provider is reminded promptly even if he/she does not log into his/her account for some days.

For purpose of illustration, when a healthcare provider logs into his/her account, he/she can view the current status of all the insurance authorization forms of his/her patients. Visual indications (e.g., different colors) can be associated with authorization forms of different status. For example, if an insurance authorization form has not been signed for a few days, it can be displayed in yellow. However, if a form has not been signed for over a week, it can be displayed in red. On the other hand, if an insurance authorization form has already been signed and sent to the appropriate recipient, if can be displayed in green.

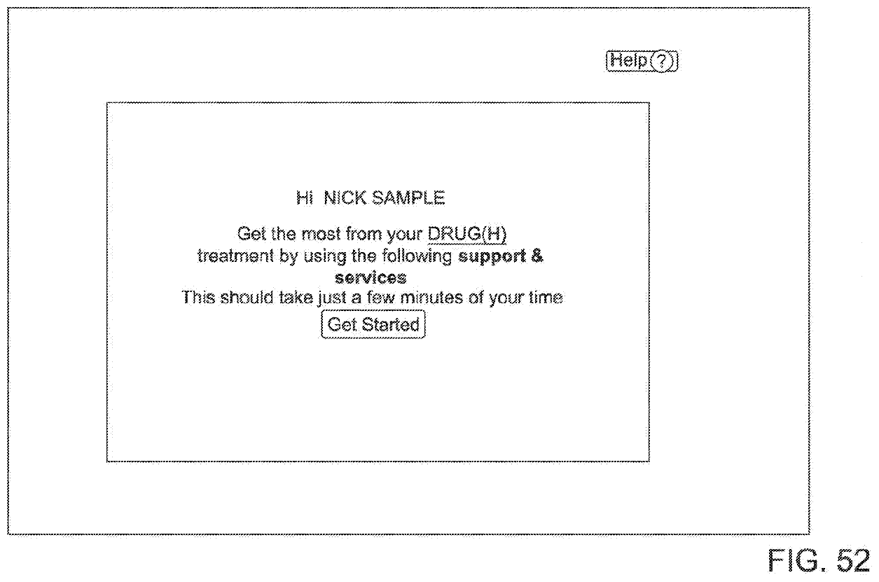

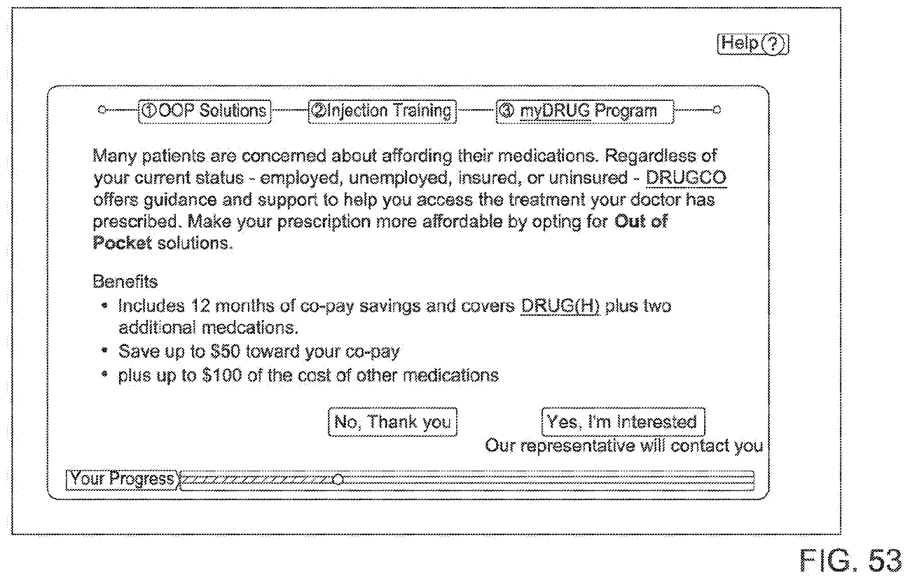

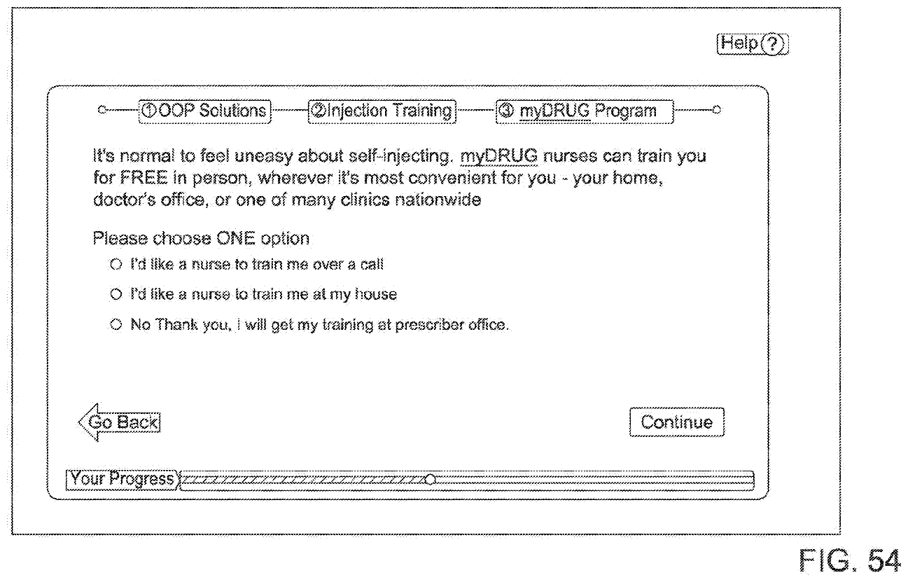

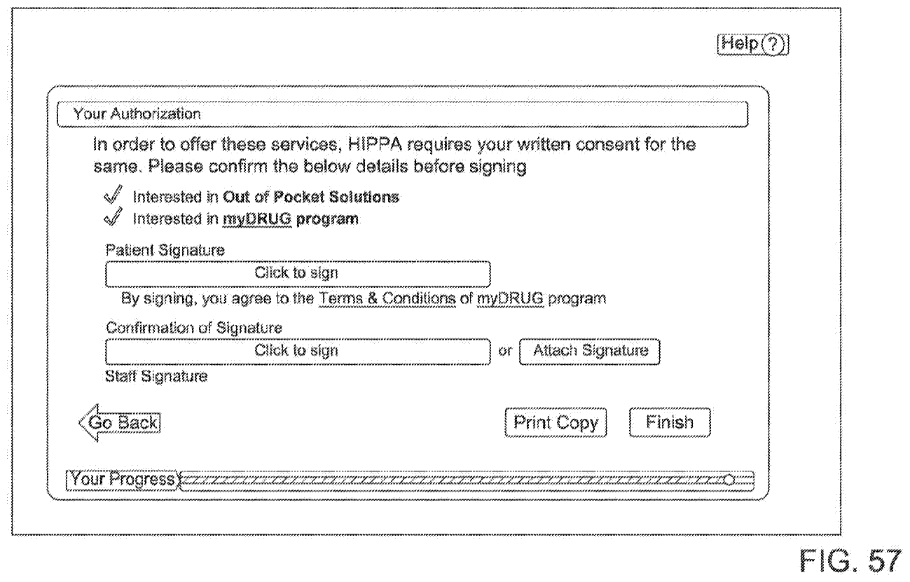

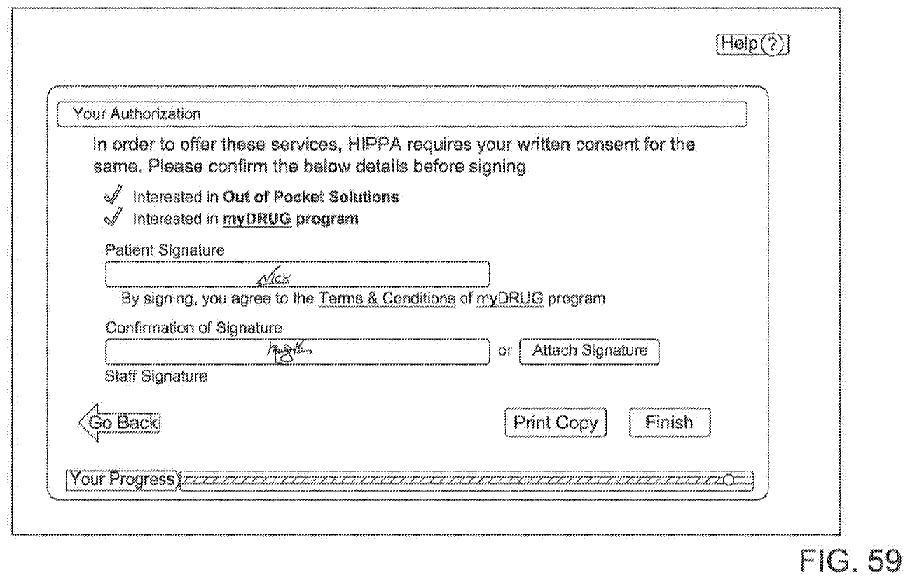

For purpose of illustration, when a patient logs into his/her account with prescription manager 7310, he/she can review information relating to his/her prescription or sign up for additional support and services, through screens provided by prescription manager 7310 as part of its user interface, as illustrated in FIG. 52. For example, FIG. 53 illustrates an example screen from which the patient can view training video on self injection. FIGS. 54-60 illustrate a series of screens that guides the patient to sign up for an optional service so that the patient can receive treatment information, training for administering the prescription medication, or the like. The patient can need to provide additional information and give various types of content (e.g., as illustrated in FIGS. 57 and 59) in order to receive these services.

For purpose of illustration, when a new medication is under clinical trial and it treats a patient's condition, prescription manager 7310 can show information about the new medication to the patient when the patient logs into his/her account. If appropriate, the patient can be asked whether he/she is willing to participate in the clinical trial, and if so, there can be screens provided that guide the patient to sign up for the clinical trial and input the necessary information.

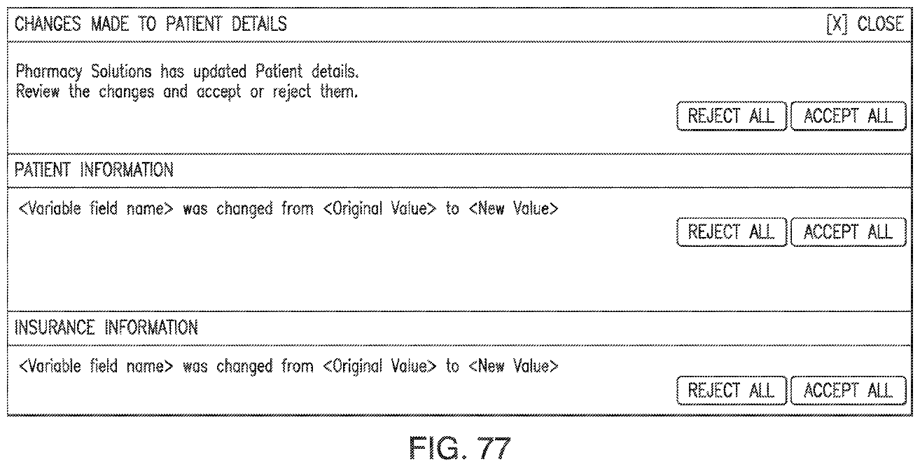

Sometimes, a patient can move from one location of residence (or work) to another location of residence (or work). While residing at the former location, the patient can have selected a nearby pharmacy or clinic as a preferred location for obtaining prescription products or services. After moving to the new location, the previously selected pharmacy or clinic can no longer be convenient for the patient and the patient can select a new pharmacy or clinic near the patient's new residential location as the preferred location for obtaining prescription products or services. For purpose of illustration, the patient can log into his/her account with prescription manager 7310 and update his/her address. The patient can also select a new pharmacy or clinic as his/her preferred pharmacy or clinic. For purpose of illustration, prescription manager 7310 can notify the patient's healthcare provider of the patient's residence move. If desired, with the patient's consent, prescription manager 7310 can help transfer the patient's current prescription and insurance approval to the newly selected pharmacy or clinic (e.g., if the newly selected pharmacy or clinic is also a part of system 7300). Additionally or alternatively, prescription manager 7310 can prompt the patient to select the closest available approved pharmacy or HCP based upon the patient's change of address as entered into prescription manager 7310. For purposes of illustration and not limitation, FIG. 77 illustrates a screen including a notification of changes made to patient details in accordance with an embodiment of the disclosed subject matter.

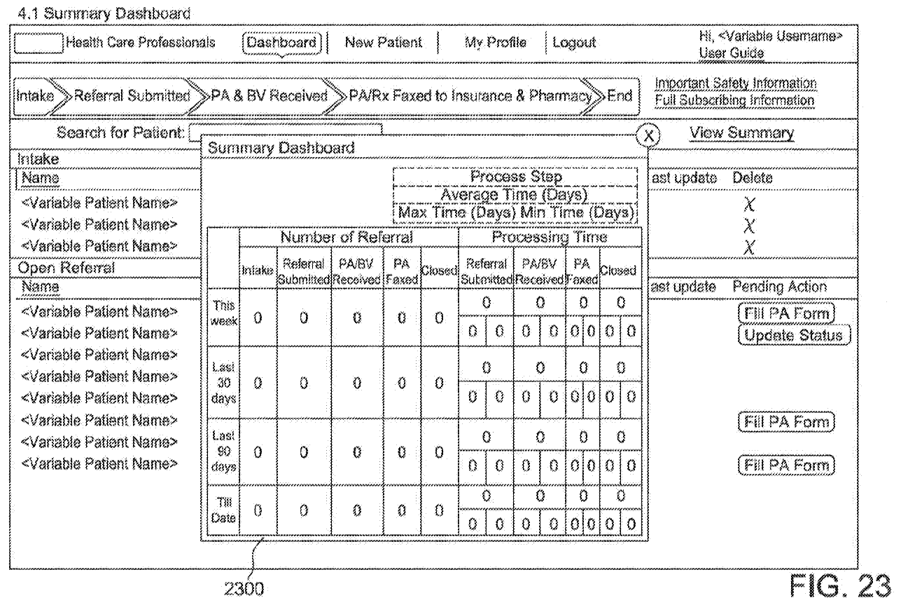

FIGS. 22 and 23 are screenshots of pages along dashboard path 706 (shown in FIG. 74C). When the user selects dashboard button 2200, dashboard window 2202 can be displayed. Dashboard window 2202 can displays overall information about the status of the cases entered into the system disclosed herein with which the user is associated. An intake section 2204 displays patients for which intake procedures have been begun, but for whom the case has not yet progressed in the system to a referral. Intake section 2204 displays the name of the patient, the date the case was created, the status of the case, and the length of time elapsed since the case was last updated. In the exemplary embodiment, the length of time elapsed is color coded to permit quick identification of the age of the elapsed time since the last update. Thus, for example, elapsed time may be colored green for cases with little time elapsed, colored yellow for cases with more than a predetermined number of days elapsed, and colored red for cases exceeding a second (and greater) predetermined number of days elapsed. In other embodiments, other color schemes may be used.

An open referral section 2206 can display patients for which an open referral exists. Open referral section 2206 displays the name of the patient, the date the case was created, the status of the case, and the length of time elapsed since the case was last updated. Open referral section 2206 can also display any pending actions needing attention of the user. The user can select to complete the pending action from the dashboard by selecting the button for the pending action the user desires to complete. In the exemplary embodiment, the length of time elapsed since the last update is color coded to permit quick identification of the time since the last update. Thus, for example, elapsed time can be colored green for cases with little time elapsed, colored yellow for cases with more than a predetermined number of days elapsed, and colored red for cases exceeding a second (and greater) predetermined number of days elapsed. In other embodiments, other color schemes may be used.

A closed section 2208 can identify closed cases with which the user is associated. Closed section 2208 displays the name of the patient, the date the case was created, and the status of the case.

From dashboard window 2202, the user can select to search for a patient using search box 2210. The user may enter complete or partial patient information, such as a last name, or unique patient identifier such as an ID number, drivers license number, insurance number, into search box 2210 and the system will return all matching patients with which the user is associated in the system. The system will not return matching patients with whom the user is not associated (e.g., the system will not return other practices' patients who match the search term entered in search box 2210). In the exemplary embodiment, the system only returns patients for whom the HCP is responsible or for whom an HCP with whom the staff member is associated is responsible. In other embodiments, the system returns search results for all patients associated with any HCP in the practice matching the search criteria.

The user can also select to view a summary dashboard from dashboard window 2202. By selecting a view summary link 2212, a dashboard summary 2300 is displayed over dashboard window 2202, as shown in FIG. 23. Dashboard summary 2300 presents a summary of the number of referrals and processing time for each step in the referral process. In other embodiments, dashboard summary 2300 may be displayed as a separate form, and not overlying dashboard window 2202.

In an exemplary embodiment, after the PA form and supporting documents are submitted to the patient's insurance provider and pharmacy, the user can continue to monitor the status of the prescription to determine when and if insurance provider approval is received and the prescription is filled. In an exemplary embodiment, the insurance provider transmits an electronic confirmation message indicating prior authorization has been granted. Accordingly, the system can track a pendency period representing the period of time between when the PA form is transmitted to the insurance provider and when the electronic confirmation message is received from the insurance provider. The pendency period and/or related metrics can be displayed on computing device 502 (shown in FIG. 5). Similarly, the pharmacy can transmit an electronic confirmation message indicating the prescription will be filled or has been filled. After the prescription is filled, the case status can be updated to closed in the system and, more specifically, in dashboard window 2202 (shown in FIG. 22). Moreover, in an exemplary embodiment, other status updates can be available. For example, a case can be updated to indicate that the insurance provider has provided the needed prior approval, updated to indicate that the prescription has actually been filled by the patient, or the like. In other embodiments, a case can be closed when the PA form and supporting documents are transmitted to the patient's insurance provider and the pharmacy. In an exemplary embodiment, a pharmacy receiver can monitor and close patient cases upon confirmation of a prescription's status. The system can additionally be communicatively coupled to an electronic medical record system, wherein updates, and documents would be transmitted to the electronic medical records system for storage and inclusion into a patient's medical record.

The system can store data concerning the prescription and fulfillment process described herein for other, non-patient specific purposes. The data can be stored in a form stripped of patient identifying information. For example, the elapsed time between submission of a referral to a pharmacy receiver and the return of a benefits verification and/or PA form can be stored for each case without inclusion of patient specific information. Data for all other time intervals in the process, e.g. between receipt of a PA form and submission of the completed PA form to an insurance provider, between submission of a PA form to an insurance provider and approval of the PA, the time between approval of the PA and filling of the prescription, or the like. The data can be collected and/or analyzed across multiple HCPs, HCP practices, pharmacy receivers, and/or filling pharmacies. However, since such data may not be generalized (i.e., it may contain identifying information) with respect to HCP, insurance provider, pharmacy receiver, and/or filling pharmacy, the data can be further analyzed to determine, for example, the diligence of various HCPs, pharmacy receivers, and/or filling pharmacies in completing their respective tasks in the system. In other embodiments, data generated by the system can be analyzed differently and/or for different purposes. If desired, HCPs can have access to the generalized data and/or the results of such analysis.

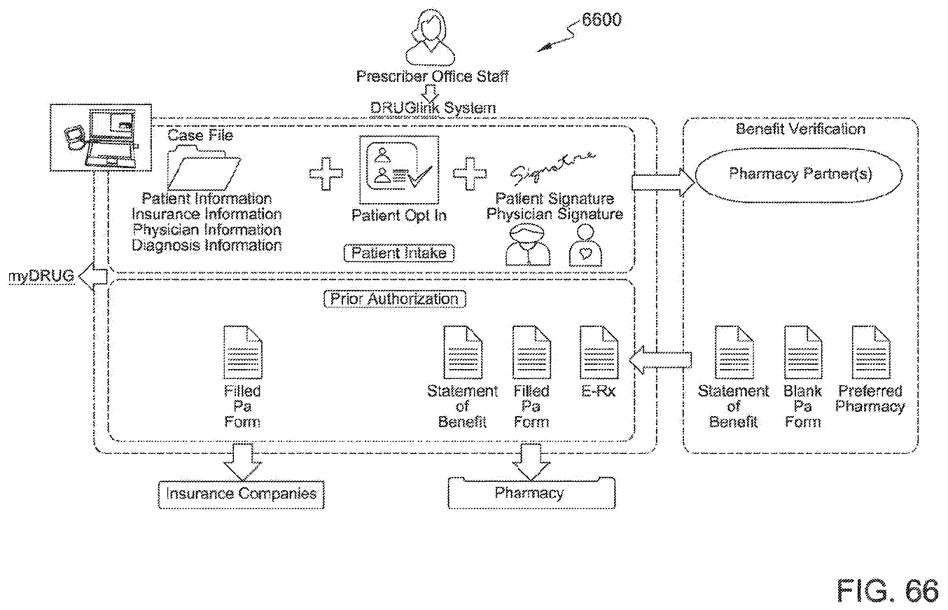

For purposes of illustration and not limitation, alternative or additional embodiments of the systems and methods disclosed herein will now be described with reference to FIGS. 66-71.

FIGS. 66-71 are block diagrams of aspects of a medical treatment coordination system 6600 in accordance with the disclosed subject matter. In FIGS. 66-71, system 6600 is used to coordinate prescribing of a pharmaceutical product, referred to herein as DRUG(H), manufactured by a pharmaceutical company, referred to herein as DRUGCO. One or more pharmacy receivers may be referred to herein as PHARMACO. A support services group for providing optional support services, such as training, information, financial aid, or the like, related to DRUG(H) is referred to by the name myDRUG.

Medical treatment coordination system 6600 is a technology platform that automates the capture of prescription information to accelerate approval, ensure greater accuracy, and connect patients to important on-boarding services. System 6600 includes several computing devices networked in communication with one another such that system 6600 extends into the offices of HCPs, to pharmacies, to insurance providers and/or other third parties such as pharmaceutical manufacturers.

Medical treatment coordination system 6600 is configured to facilitate patient awareness of patient services associated with the managed drug including for example, a patient's ability to afford their medication and self-injection. Medical treatment coordination system 6600 permits a manufacturer of the managed drug to contact new patients, with the patient's informed consent, to improve both the awareness and use of the services including Prescription Protection (PP), the injection instruction service and other myDRUG services.

Medical treatment coordination system 6600 includes a computer tablet, a keyboard, and an optical scanner device. The hardware is installed in physician offices under a user agreement between the drug manufacturer and the practice. In various embodiments, this platform exclusively runs a web-based software program that allows healthcare providers (HCPs) and patients to enter all data required to complete a valid prescription, prior authorization, and patient consent for secure on-boarding services from the drug manufacturer. Medical treatment coordination system 6600 provides an integrated data collection system that permits the healthcare provider to reuse previously entered data to streamline HCP operations. All systems are HIPAA-validated to ensure privacy and comply with all pharmacy requirements.

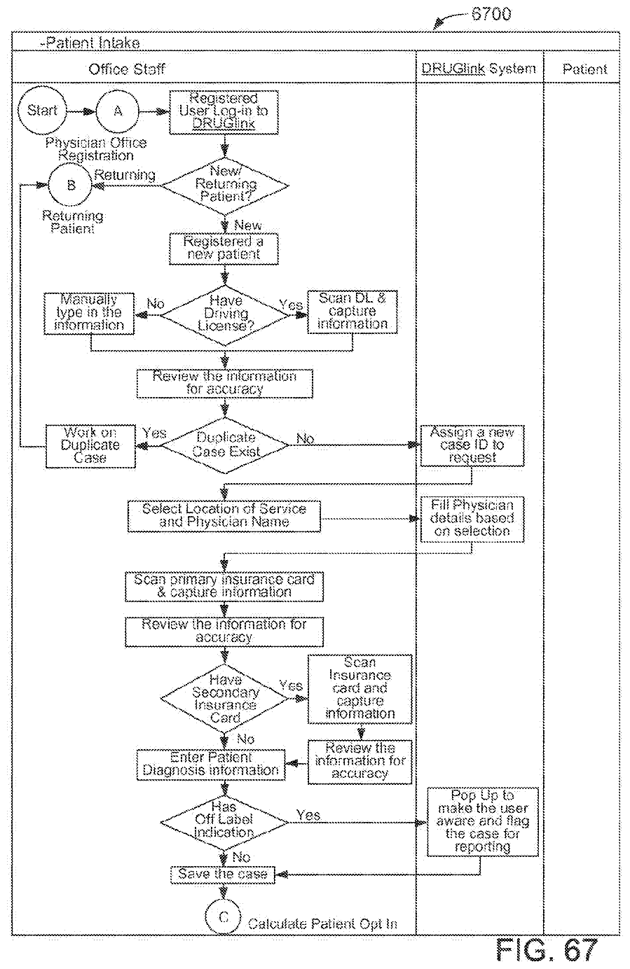

FIG. 67 is a flow diagram of a patient intake process 6700 using medical treatment coordination system 6600 in accordance with an exemplary embodiment of the disclosed subject matter. In the exemplary embodiment, process 6700 is configured to capture patient information from a drivers' license or other identification and/or insurance card.

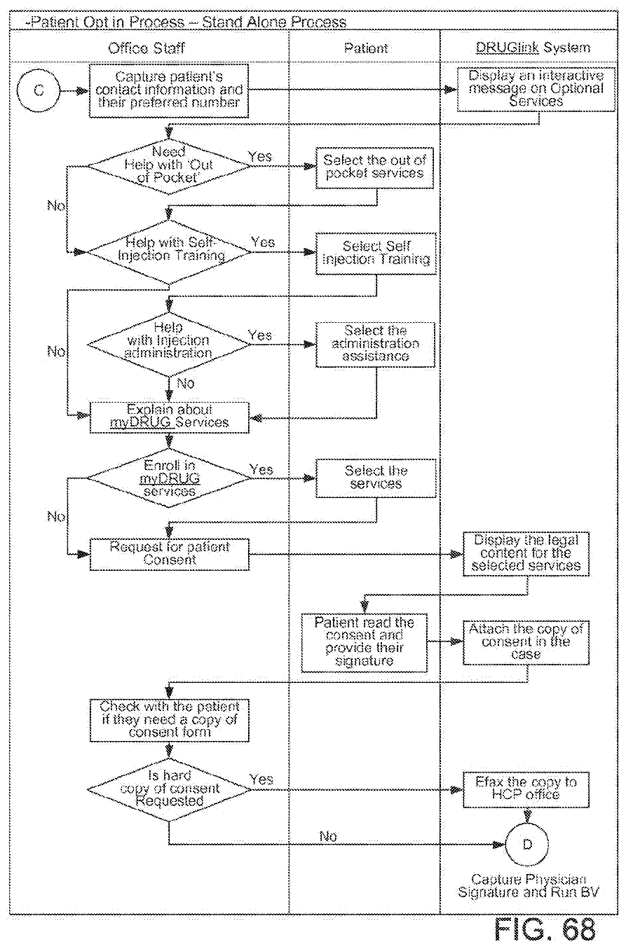

FIG. 68 is a flow diagram of a patient opt-in process illustrating steps to permit a patient to opt-in to myDRUG services and capture a patient signature using medical treatment coordination system 6600.

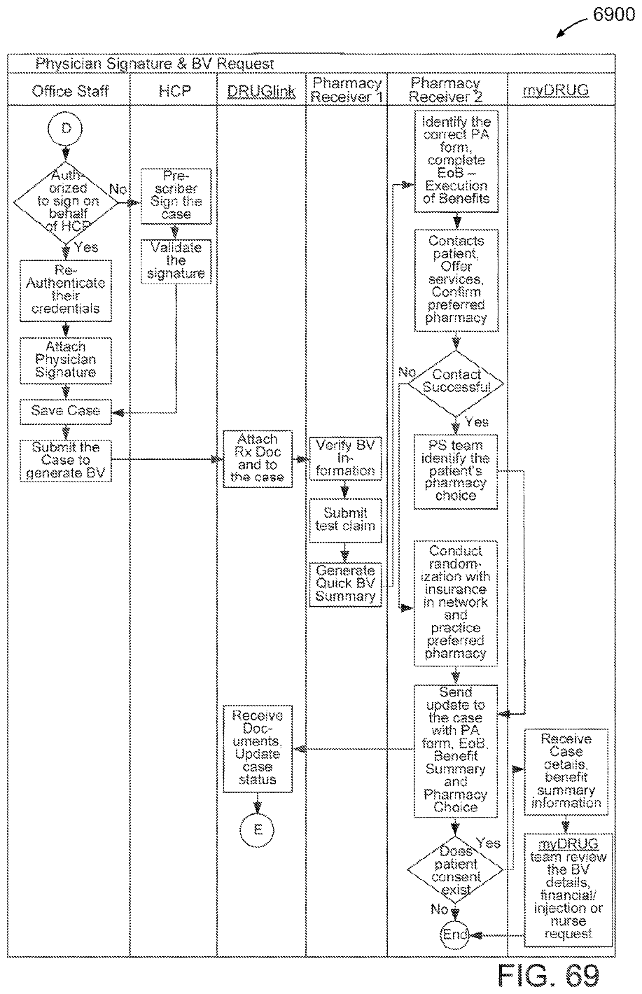

FIG. 69 is a flow diagram of a process 6900 configured to generate a benefit verification (BV) and a digital rendering of a paper prescription and/or an electronic prescription (E-Prescription) using medical treatment coordination system 6600. In the exemplary embodiment, when the case information and patient signature processes have been completed, the prescriber signs the case to generate a BV. In an exemplary embodiment, a signed BV is or includes a digital rendering of a paper prescription and/or an E-Prescription, and is forwarded to a pharmacy of the patient's choice.

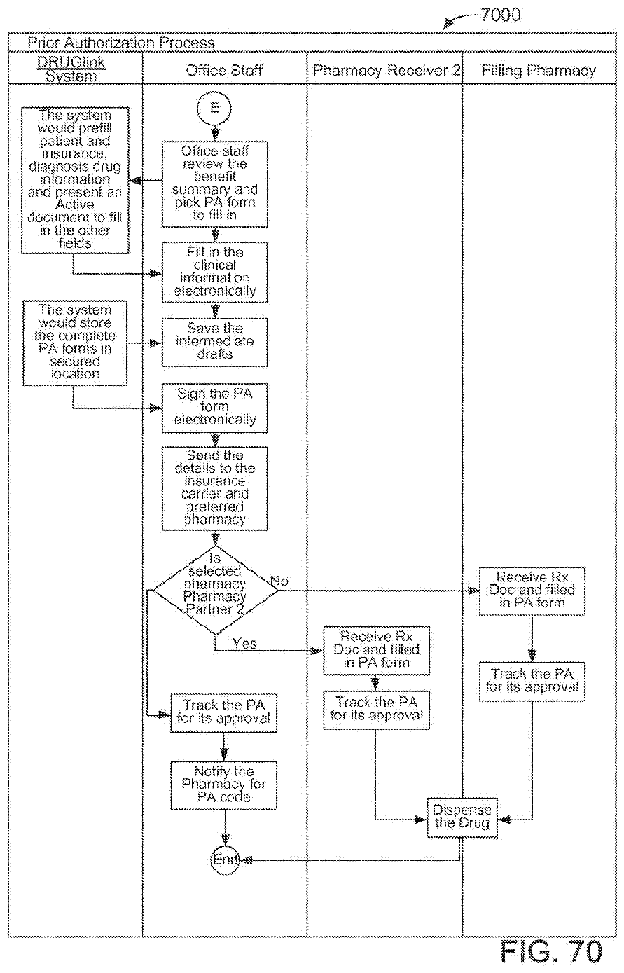

FIG. 70 is a flow diagram of a prior authorization (PA) process 7000 illustrating the steps involved in filling in the prior authorization form and faxing the PA form to an insurance company. After BV submission, medical treatment coordination system 6600 returns the BV and proper PA form. The office staff may populate the PA form with previously entered data.

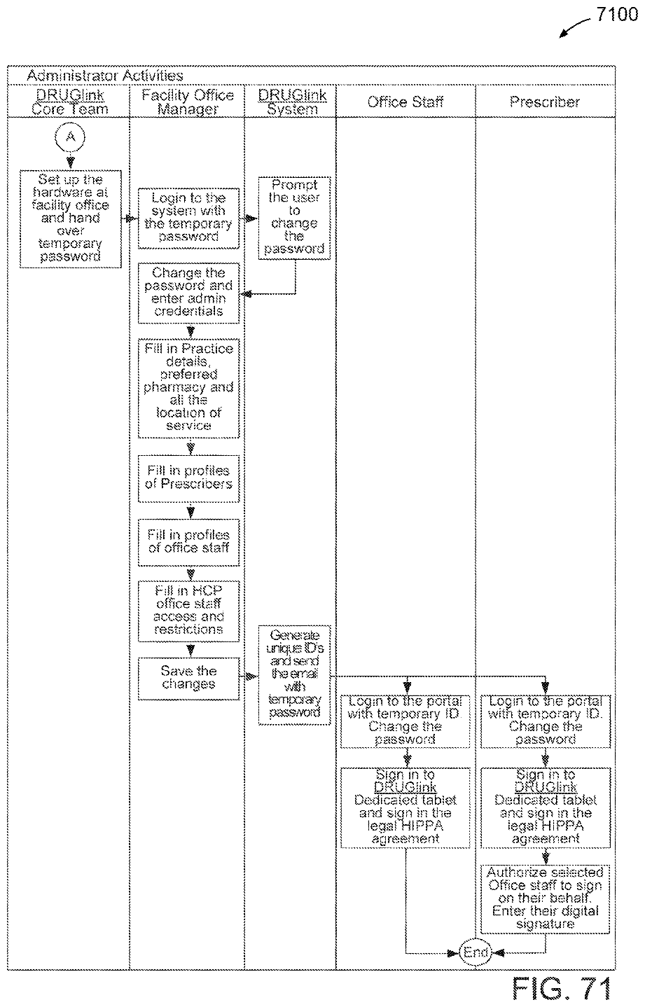

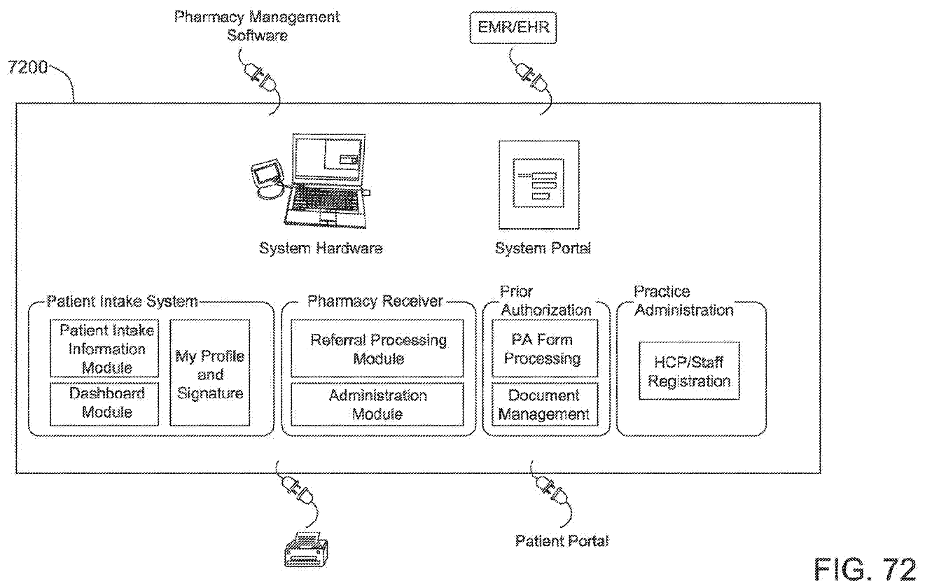

FIG. 71 is a flow diagram of administrator activities 7100 to register a facility, staff member, and physician into medical treatment coordination system 6600. This flow is followed once for each facility.

The exemplary system 6600 can be used for any particular prescription product or service. Moreover, medical treatment coordination system 6600 can be used in connection with a number of different prescription products and/or services. In such embodiments, a user can select which drug system 6600 is being used with, i.e. which product or service is being prescribed or ordered, in each instance.

As embodied herein, medical treatment coordination system 6600 can integrate services provided by E-Prescription, Prior Authorization, and Patient On-Boarding services and improves the patient on-boarding rates by offering the benefits of reduced paperwork and reliable PA form completion to clinics. Medical treatment coordination system 6600 enables a higher patient opt-in to myDRUG program resulting in decreased abandonment of prescriptions at the pharmacy, improved patient compliance and consistency due to training and follow-up, and an increased use of proper starting dose.

One of ordinary skill in the art will appreciate that the exemplary screenshots depicted in the figures and described herein are provided for purposes of example and not limitation. Accordingly, the sequence and grouping of like screenshots can be modified as desired. For purpose of illustration and not limitation, FIGS. 82-260 of U.S. Provisional Application No. 61/712,153, which is incorporated herein by reference in its entirety, provide alternative screenshots in accordance with an exemplary embodiment of the disclosed subject matter.

The disclosure is described as applied to certain exemplary embodiments, including, systems and methods for facilitating and/or coordinating a medical order/prescription of a prescription product. As used herein, a medical treatment can include, but is not limited to, any medical product and/or medical service provided to a patient that requires a prescription and that may also require prior authorization from an insurance provider. Thus, a medical treatment may include drugs, pharmaceutical products, medical devices, medical therapies, physical therapy, medical supplies, or the like. Further, as used herein, a medical order/prescription can include an order, request, instruction, and/or recommendation for a medical treatment. Although the system and process described herein relates generally to facilitating and/or coordinating a medical order/prescription of a prescription product, specific embodiments of the disclosed subject matter can be used in connection with prescribing a prescription drug known as the HUMIRA® product, also known generically as adalimumab. (HUMIRA is a registered trademark of Abbott Biotechnology Ltd., Hamilton, Bermuda.) For example, indications, diagnoses, specialties of physicians, dosing, delivery routes, or the like are described herein in the context of prescribing and obtaining prior authorization for the HUMIRA® product for a patient. However, the systems and processes described herein may also be used with any other medical treatment, including other prescription drugs, and are not limited to use in connection with the HUMIRA® product.

Embodiments of the presently disclosed subject matter as described herein relate to medical treatment management methods and systems. The methods and systems described can be used to facilitate, coordinate, or manage medical treatments such as medical services and/or medical products. As used herein, medical treatments include any suitable medical service or medical product. Medical products include physical devices that may be used or consumed by a patient in the course of their medical treatment. Medical services include activities that support the supply or operation of medical devices or standalone services that serve as treatment, for example, but not limited to, counseling. Medical services may include, for example, one or more services related to a medical product, a pharmaceutical product, and/or a medical treatment. Moreover, medical services may also be used to facilitate education and/or training related to a particular pharmaceutical and/or healthcare in general.

The methods and systems described herein may be implemented using computer programming or engineering techniques including computer software, firmware, hardware or an combination or subset thereof, wherein the technical effect may include at least one of: (a) receiving patient data from a healthcare provider (HCP) including a completed prescription form for the pharmaceutical product for the patient, and insurance data identifying an insurance provider of the patient, (b) storing the patient data and the insurance data within a memory device, (c) determining that an insurance provider requires a prior authorization the prescription as a prerequisite to covering a claim by the patient for the pharmaceutical product, (d) determining a current electronic prior authorization form required by the insurance provider for requesting the prior authorization for the prescription, and (e) transmitting the determined prior authorization form to the HCP, wherein the HCP is prompted to complete the determined prior authorization form by automatically populating at least one data field included within the determined prior authorization form with patient data stored within the memory device, and transmitting the determined prior authorization form completed by the HCP to the insurance provider.

Certain exemplary systems, comprise a healthcare provider (HCP) technology platform to automate the capture of prescription information, HCP information, insurance information, and/or patient information to facilitate accelerated prescription approval. Other features of the system include greater accuracy of information, and an ability to connect patients to optional services related to the prescribed medicine or to financial services, which may be available to assistance the patient in paying for the treatments.

As used herein, an HCP includes a person who provides medical services or generates valid prescriptions, and includes entities such as medical practices that include one or more medical doctor. HCP information may include identifying information, such as name, address, phone number, license numbers, and DEA numbers associated with the HCP, the HCP's employees, and others associated with the HCP. Insurance information may include information concerning an insurance company and/or a policy issued by the insurance company including, for example, name, address and contact information for the insurer, name of the insured, policy number(s), deductibles, and co-pays. Patient information may include identifying personal information concerning a patient, such as name, address, phone number, email address, driver's license numbers, and social security numbers.

Unless otherwise indicated, the functions described herein may be performed by executable code and instructions stored in computer readable memory and running on one or more processor-based systems. However, state machines, and/or hardwired electronic circuits can also be utilized. Further, with respect to the example processes described herein, not all the process states need to be reached, nor do the states have to be performed in the illustrated order.

The term processor, as used herein, refers to central processing units, microprocessors, microcontrollers, reduced instruction set circuits (RISC), application specific integrated circuits (ASIC), logic circuits, and any other circuit or processor capable of executing the functions described herein.

As will be appreciated based on the foregoing specification, the above-described embodiments of the disclosure may be implemented using computer programming or engineering techniques including computer software, firmware, hardware or any combination or subset thereof, wherein a technical effect is one or more of receiving patient data comprising a prescription for a pharmaceutical product for a patient and an identification of the patient's insurance provider, determining whether or not a prior authorization from the patient's insurance provider is needed before the prescription may be filled, and providing a prior authorization form to the patient's healthcare provider if prior authorization from the patient's insurance provider is needed. Any such resulting program, having computer-readable code means, may be embodied or provided within one or more computer-readable media, thereby making a computer program product, i.e., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium such as the Internet or other communication network or link. The article of manufacture containing the computer code may be made and/or used by executing the code directly from one medium, by copying the code from one medium to another medium, or by transmitting the code over a network.

It should be understood that the systems and methods described herein can likewise be used with any drug, pharmaceutical product or service, medical device, and/or with any other products or services that may or may not require a prescription, such as an order for a medical procedure or the like.

Herein, an element or step recited in the singular and preceded with the word “a” or “an” should be understood as not excluding plural elements or steps, unless such exclusion is explicitly recited. Furthermore, references to “one embodiment” are not intended to be interpreted as excluding the existence of additional embodiments that also incorporate the recited features.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

This disclosure encompasses all changes, substitutions, variations, alterations, and modifications to the example embodiments herein that a person having ordinary skill in the art would comprehend. Moreover, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative.

Moreover, although this disclosure describes and illustrates respective embodiments herein as including particular components, elements, functions, operations, or steps, any of these embodiments may include any combination or permutation of any of the components, elements, functions, operations, or steps described or illustrated anywhere herein that a person having ordinary skill in the art would comprehend.

1. A prescription manager computing device for facilitating a medical order/prescription of a prescription product for a patient covered by a provider, the prescription manager computing device communicatively coupled to a healthcare provider (HCP) computing device and a benefits verifier computing device over a network, the prescription manager computing device comprising:
at least one memory device; and
a processing device communicatively coupled to the at least one memory device, the processing device configured to:
receive, over the network, prescription product information for the prescription product from the HCP computing device;
receive, over the network, patient intake information for the patient including provider information for the patient from the HCP computing device;
generate a benefits verification request for the patient based on the patient intake information;
receive, over the network, a benefits summary related to the patient from the benefits verifier computing device, the benefits summary generated by the benefits verifier computing device based on the benefits verification request;
receive, over the network, prior authorization data from the benefits verifier computing device, the prior authorization data identifying prior authorization requirements associated with the medical order/prescription;
generate a data message that includes the benefits summary and the prior authorization data;
transmit the data message to the HCP computing device to cause the benefits summary and the prior authorization data to be displayed on the HCP computing device to enable review of the benefits summary by the HCP, and to enable completion of a prior authorization form by the HCP;
generate a patient information dashboard, wherein the patient information dashboard includes status information that indicates i) whether the HCP computing device has received the benefits summary and ii) whether the prior authorization form has been at least one of completed, submitted to the provider, and approved by the provider, the patient information dashboard listing the status information in association with the patient;
cause the patient information dashboard to be displayed on the HCP computing device;
monitor receipt of the benefits summary and processing of the prior authorization form;
generate an update to the status information based on the monitoring; and
cause the patient information dashboard displayed on the HCP computing device to reflect the update to the status information.

2. The prescription manager computing device of claim 1, wherein the at least one processing device is further configured to, prior to transmitting the data message to the HCP computing device:
encrypt the data message to protect patient privacy and to secure the data message for transmission to the HCP computing device; and
compress the data message to reduce data size and to facilitate transmission of the data message to the HCP computing device.

3. The prescription manage computing device of claim 1, wherein the at least one processing device is configured to transmit the data message at a first time, and wherein the at least one processing device is further configured to:
determine a current time;
calculate an elapsed time as the difference between the first time and the current time, the elapsed time indicating how long ago the data message including the benefits summary and the prior authorization data was transmitted to the HCP computing device; and
store the calculated elapsed time in the at least one memory device to facilitate tracking the benefits summary and completion of the prior authorization requirements.

4. The prescription manager computing device of claim 1, wherein causing the patient information dashboard to be displayed on the HCP computing device enables a user of the HCP computing device to view the current status without having to access the actual benefits summary.

5. The prescription manager computing device of claim 1, wherein facilitating the medical order/prescription includes facilitating execution of a medical order/prescription of the prescription product or facilitating approval of a payment for the prescription product.

6. The prescription manager computing device of claim 1, wherein the provider includes an insurance carrier, a governmental agency, or a third party payor.

7. The prescription manager computing device of claim 1, wherein the processor is further configured to transmit a request for additional patient information to the HCP computing device over the network.

8. The prescription manager computing device of claim 1, wherein the processor is further configured to generate a prescription document from at least a portion of the prescription product information and the patient intake information and release the prescription document to a pharmacy.

9. A method for facilitating a medical order/prescription of a prescription product for a patient covered by a provider, the method performed using a prescription manager computing device communicatively coupled to a healthcare provider (HCP) computing device and a benefits verifier computing device over a network, the prescription manager computing device including at least one memory device and a processing device communicatively coupled to the at least one memory device, the method comprising:
receiving, over the network, prescription product information for the prescription product from the HCP computing device;
receiving, over the network, patient intake information for the patient including provider information for the patient from the HCP computing device;
generating a benefits verification request for the patient based on the patient intake information;
receiving, over the network, a benefits summary related to the patient from the benefits verifier computing device, the benefits summary generated by the benefits verifier computing device based on the benefits verification request;
receiving, over the network, prior authorization data from the benefits verifier computing device, the prior authorization data identifying prior authorization requirements associated with the medical order/prescription;
generating a data message that includes the benefits summary and the prior authorization data;
transmitting the data message to the HCP computing device to cause the benefits summary and the prior authorization data to be displayed on the HCP computing device to enable review of the benefits summary by the HCP, and to enable completion of a prior authorization form by the HCP;
generating a patient information dashboard, wherein the patient information dashboard includes status information that indicates i) whether the HCP computing device has received the benefits summary and ii) whether the prior authorization form has been at least one of completed, submitted to the provider, and approved by the provider, the patient information dashboard listing the status information in association with the patient;
causing the patient information dashboard to be displayed on the HCP computing device;
monitoring receipt of the benefits summary and processing of the prior authorization form;
generating an update to the status information based on the monitoring; and
causing the patient information dashboard displayed on the HCP computing device to reflect the update to the status information.

10. The method of claim 9, further comprising, prior to transmitting the data message to the HCP computing device:
encrypting the data message to protect patient privacy and to secure the data message for transmission to the HCP computing device; and
compressing the data message to reduce data size and to facilitate transmission of the data message to the HCP computing device.

11. The method of claim 9, wherein transmitting the data message comprises transmitting the data message at a first time, and wherein the method further comprises:
determining a current time;
calculating an elapsed time as the difference between the first time and the current time, the elapsed time indicating how long ago the data message including the benefits summary and the prior authorization data was transmitted to the HCP computing device; and
storing the calculated elapsed time in the at least one memory device to facilitate tracking the benefits summary and completion of the prior authorization requirements.

12. The method of claim 9, wherein causing the patient information dashboard to be displayed on the HCP computing device enables a user of the HCP computing device to view the current status without having to access the actual benefits summary.

13. The method of claim 9, wherein the provider includes an insurance carrier, a governmental agency, or a third party pay or.

14. The method of claim 9, further comprising:
transmitting a request for additional patient information to the HCP computing device over the network.

15. A non-transitory computer readable medium containing computer-executable instructions that when executed cause a prescription manager computing device to perform a method of facilitating a medical order/prescription of a prescription product for a patient covered by a provider, the prescription manager computing device communicatively coupled to a healthcare provider (HCP) computing device and a benefits verifier computing device over a network, the prescription manager computing device including at least one memory device and a processing device communicatively coupled to the at least one memory device, the method comprising:
receiving, over the network, prescription product information for the prescription product from the HCP computing device;
receiving, over the network, patient intake information for the patient including provider information for the patient from the HCP computing device;
generating a benefits verification request for the patient based on the patient intake information;
receiving, over the network, a benefits summary related to the patient from the benefits verifier computing device, the benefits summary generated by the benefits verifier computing device based on the benefits verification request;
receiving, over the network, prior authorization data from the benefits verifier computing device, the prior authorization data identifying prior authorization requirements associated with the medical order/prescription;
generating a data message that includes the benefits summary and the prior authorization data;
transmitting the data message to the HCP computing device to cause the benefits summary and the prior authorization data to be displayed on the HCP computing device to enable review of the benefits summary by the HCP, and to enable completion of a prior authorization form by the HCP;
generating a patient information dashboard, wherein the patient information dashboard includes status information that indicates i) whether the HCP computing device has received the benefits summary and ii) whether the prior authorization form has been at least one of completed, submitted to the provider, and approved by the provider, the patient information dashboard listing the status information in association with the patient;
causing the patient information dashboard to be displayed on the HCP computing device;
monitoring receipt of the benefits summary and processing of the prior authorization form;
generating an update to the status information based on the monitoring; and
causing the patient information dashboard displayed on the HCP computing device to reflect the update to the status information.

16. The non-transitory computer readable medium of claim 15, wherein the method further comprises, prior to transmitting the data message to the HCP computing device:
encrypting the data message to protect patient privacy and to secure the data message for transmission to the HCP computing device; and
compressing the data message to reduce data size and to facilitate transmission of the data message to the HCP computing device.

17. The non-transitory computer readable medium of claim 15, wherein transmitting the data message comprises transmitting the data message at a first time, and wherein the method further comprises:
determining a current time;
calculating an elapsed time as the difference between the first time and the current time, the elapsed time indicating how long ago the data message including the benefits summary and the prior authorization data was transmitted to the HCP computing device; and
storing the calculated elapsed time in the at least one memory device to facilitate tracking the benefits summary and completion of the prior authorization requirements.

18. The non-transitory computer readable medium of claim 15, wherein causing the patient information dashboard to be displayed on the HCP computing device enables a user of the HCP computing device to view the current status without having to access the actual benefits summary.

19. The non-transitory computer readable medium of claim 15, wherein the provider includes an insurance carrier, a governmental agency, or a third party payor.

20. The non-transitory computer readable medium of claim 15, wherein the method further comprises:
transmitting a request for additional patient information to the HCP computing device over the network.
